(19) United States
(12) Patent Application Publication
(10) Pub. No.: US 2024/0317780 A1
(43) Pub. Date: Sep. 26, 2024

(54) RADIOPHARMACEUTICAL SOMATOSTATIN RECEPTOR LIGANDS AND PRECURSORS THEREOF

(71) Applicant: Technische Universität München, München (DE)

(72) Inventors: Hans-Jürgen Wester, Schweitenkirchen (DE); Mara Parzinger, Bichl (DE); Markus Frederik Fahnauer, München (DE)

(21) Appl. No.: 18/290,061

(22) PCT Filed: May 12, 2022
(86) PCT No.: PCT/EP22/62985

§ 371 Date: Nov. 9, 2023

(30) Foreign Application Priority Data
May 14, 2021 (EP) 21173849.7

(51) Int. Cl. C07F 7/12; A61K 51/04; A61K 51/08

(52) U.S. Cl. CPC C07F 7/12; A61K 51/0497; A61K 51/083; A61K 51/088

(57) ABSTRACT

Provided are novel SST receptor ligand compounds suitable for the imaging and/or the treatment of neuroendocrine tumors. These SST receptor ligand compounds are comprised of a SST binding motif, a silicon-fluoride acceptor group which can be labeled with ¹⁸F by isotopic exchange of ¹⁹F by ¹⁸F or which is labeled with ¹⁸F, a chelating group suitable for forming a chelate with a radioactive or non-radioactive cation, and a hydrophilic amino acid unit or a sequence of such units.

Neuroendocrine tumors (NETs) are a heterogenous group of malignancies, originating from the neuroendocrine system. This system is comprised of neuroendocrine cells in a variety of different tissues like endocrine glands (pituary, parathyroids, adrenal), pancreatic tissue or the endocrine cells located in the digestive and respiratory system (diffuse endocrine system: lungs, gastrointestinal tract) [1]. NETs are a rare entity with an incidence of 2-5/100000 (0.5% of newly diagnosed malignancies per year), depending on the patients (ethnic) decent. With 67%, tumors of the gastrointestinal tract are the most common, followed by NETs in the respiratory system with 25%. Even though the incidence may be low, the number of diagnosed entities has increased over the past 30 years due to optimized methods in diagnostics [1-4]. However, somatostatin receptors (SST) expression are not restricted to neuroendocrine lung tumors, but are also a feature of some non-neuroendocrine carcinomas [5, 6].

Shortly after cloning, two classes or groups of SST receptors were identified on the basis of their phylogeny, structural homologies, and pharmacological properties: a) the first class was referred to as SRIF1, comprising SST2, SST3, and SST5 receptor subtypes, whereas the other class was referred to as SRIF2, comprising the other two recombinant receptor subtypes SST1 and SST4 [7]. The G-protein-coupled receptors SST₁₅ are expressed naturally on neuroendocrine cells of various tissues but are overexpressed on various types of NETs and other tumors and their metastases [8-10]. Therefore, the SST receptors are attractive targets for diagnostic clarification, applying e.g. positron-emission-tomography (PET) [11]. The high expression density and frequency of SST₂ was found to play a dominant role for the diagnosis and therapy of cancer, resulting in the development of radiopharmaceuticals with generally high affinity towards SST₂ and additional affinity of various degree to the other receptors [8, 11]. In addition, pan-ligands with high affinity to all 5 subtypes have been developed [6].

In the past decades, positron emission tomography (PET) has developed into a leading imaging method in nuclear medicine [12]. In contrast to computed tomography and magnetic resonance tomography, which provides detailed anatomical and morphological information, PET provides functional information, i.e. about physiological and biochemical processes in the body before any macroscopic or morphological abnormalities or clinical signs of possible disease appear [13].

For PET diagnostics, ⁶⁸Ga and ¹⁸F are currently the most often used β⁺ (positron) emitting radionuclides. For the complexation of gallium, chelators like DOTA or NOTA are available. A new methodology for introducing fluorine via an isotope exchange reaction using silicon fluoride acceptors (SiFAs) can significantly improve the accessibility of 18F-labeled ligands [14]. As a PET nuclide ⁶⁸Ga shows some beneficial properties. Although ⁶⁸Ga can also be produced by means of on-site cyclotrons, the broad availability of ⁶⁸Ga is ensured by means of commercially available, easy to handle ⁶⁸Ge/⁶⁸Ga generators (⁶⁸Ge; t_{1/2}=270.8 d). Such generators are often expensive and typically provide up to 1.8 GBq ⁶⁸Ga per elution. The daughter radionuclide ⁶⁸Ga emits β⁺ with a high abundance (89%) and a positron energy (Eₘₐₓ(β⁺)=1.89 MeV) that allows good resolution on clinical scanners. The physical half-life of 68 min provides sufficient time for labeling, while labeling is simple and can be carried out by complexation with high radiochemical yields (RCY).

Because of its nuclear properties, ¹⁸F has always been the PET-radionuclide of choice. Compared to ⁶⁸Ga, ¹⁸F has a higher positron abundance (97% β⁺), a weaker β⁺-emission energy (Eₘₐₓ(β⁺)=0.64 MeV) and a longer half-life (t_{1/2}=109.8 min). This provides image images with better resolution, especially in combination with small animal scanners, but also on clinical scanners. Unlike ⁶⁸Ga, the production of ¹⁸F requires an on-site cyclotron or the daily supply with ¹⁸F-fluoride from a cyclotron site. Modern cyclotrons allow for the production of >10 ci (370 GBq) ¹⁸F-fluoride, which in turn allows for the large scale production of ¹⁸F-radiopharmaceuticals and the treatment of a large number of patients and with significantly reduced costs [12].

For the introduction of ¹⁸F into a tracer by formation a of carbon-fluorine bond, most often a nucleophilic substitution, either aliphatic or aromatic, of a leaving group by [¹⁸F]fluoride is carried out. Because of the low reactivity, an activation of the fluoride anion in anhydrous, polar aprotic solvents as well as elevated reaction temperatures is typically required. Thus, eliminations are often observed resulting in side reactions and undesired by-products, which require purification steps and lead to reduced RCY [15].

To circumvent the problems of classical nucleophilic fluorination, new methods for the formation of phosphorus-fluorine, silicon-fluorine, or boron-fluorine bonds have been developed. One promising method is the ¹⁸F labeling of silicon fluoride acceptors by isotopic exchange reactions [16]. The focus here has been placed on silicon-fluorine compounds, which have a high stability under physiological conditions (pH 7.4 in blood plasma), but also allow a rapid isotope exchange at room temperature. The compound di-tert-butylphenyl-fluorosilane and derivatives thereof proved to be particularly stable against defluorination in fluoride-free aqueous solution. The stability is based on a shielding effect of the two sterically demanding tert-butyl substituents that efficiently inhibit the attack of a hydroxyl group at the Si to form the pentacoordinated intermediate and finally the S_{N2} based OH-for-F-substitution of such compounds [14].

In contrast, the two tert-butyl-groups are not able to prevent the attack of a small fluoride anion and the following SN2 based ¹⁸F-for-¹⁹F-isotopic exchange. The ¹⁹F-¹⁸F isotope exchange is rapid, even at room temperature, and result in 80-90% RCY within 10 to 15 min at room temperature when ¹⁸F-fluoride of high specific activity (>100 GBq/μmol) is used [14]. Thus, di-tert-butylphenyl-fluorosilane and analogues thereof have been named “Silicone-Based Fluoride Acceptors” (SiFA). The first compounds with SiFA motifs for effective coupling to peptides were para-(di-tert-butylfluorosily)benzoic acid (SiFA-benzoic acid) and para-(di-tert-butylfluorosily)benzaldehyde (SiFA-benzaldehyde). The SiFA-benzaldehyde can be attached to a peptide via oxime ligation, while the SiFA-benzoic acid is converted into an active ester or activated in-situ to act as an acylation agent on an N-terminal end of a peptide or a side chain amine of a peptide to form a peptide bond [17]. A recently published approach reported by Niedermoser et. al. described the development of SiFAlinTATE, a SiFA based Tyr³-Octreotate analogue labelled with ¹⁸F by means of the SiFAlin aldehyde moiety [18, 19]. The above mentioned ¹⁸F-based SST tracer [¹⁸F]SiFAlinTATE has gained some interest over the last years [20, 21]. However, compared to clinically established ⁶⁸Ga-based ligands like [⁶⁸Ga]DOTA-TATE, the overall in vivo characteristics were less favorable [19, 20, 22].

One significant drawback of oxime based SiFA-radiopharmaceuticals that are produced via aldehydes synthesized is the equilibrium between the oxime product on one hand, and the educts—the free SiFA-aldehyde or SiFA/in-aldehyde and the aminooxy-conjugated peptide-one the other hand, in aqueous solution at pH 1.5-4.5. Consequently and not surprisingly, the dissolution of the SiFAlin precursor in a solution with oxalic acid (which is necessary to adjust the alkaline pH) prior to the isotopic exchange with preactivated [¹⁸F]fluoride, has been found to result in decomposition of the precursor [23].

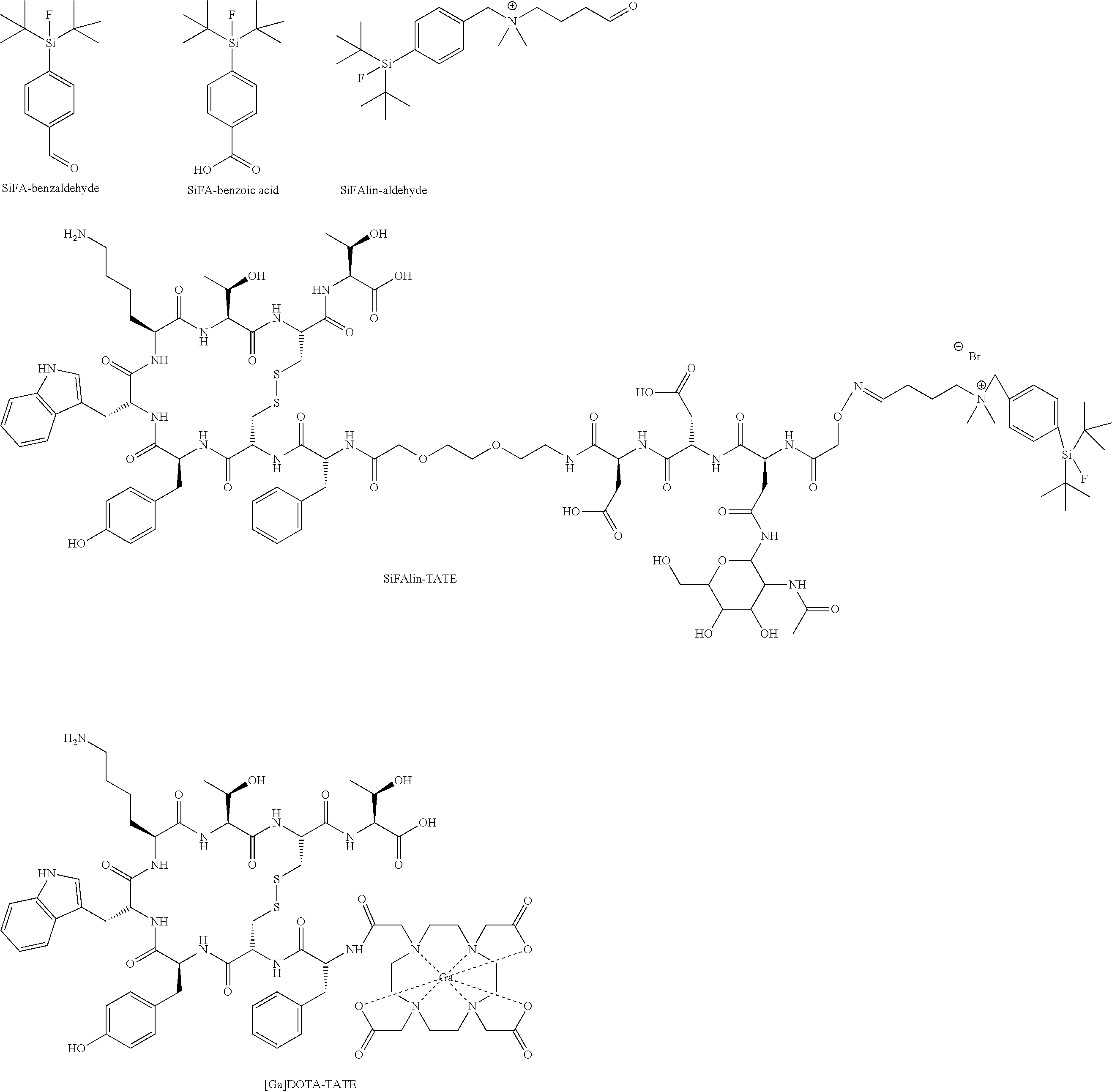

The invention thus provides novel SiFA-based SST receptor ligand compounds suitable for the imaging and/or treatment of neuroendocrine tumors. These SST receptor ligand compounds are comprised of: 1. a SiFA-moiety, 2. a chelator or a chelate and 3. a hydrophilic amino acid/amino acid sequence. The latter two compensate the high lipophilicity and partially binding to human serum albumin (HSA) of the SiFA-building block as relevant factors influencing in vivo parameters like blood clearance, extravasation and diffusion into tissue, such as tumor tissue, or the route of excretion, to mention the most relevant. Additionally, the amino acid/amino acid sequence modulates the overall ligand net charge, influencing the ligands affinity towards the target. Through adjustment of the SiFA-building block (e.g. addition of a positive charge) the affinity of SiFA to HSA can be lowered distinctively. All those parameters—lipophilicity, affinity to target and HSA-binding—affect the tumor uptake of such radiopharmaceuticals and therefore the diagnostic accuracy or therapeutic effect. In addition, the above described design not only allows the development of ¹⁸F-labeled diagnostic radiopharmaceuticals, but also allows for the development of therapeutic tracers labelled at the chelator with a radiometal (such as Lu-177, Y-90 or Ac-225, to mention only a few) to form a chelate. While in such therapeutic tracers the SiFA moiety is not radioactive ( . . . Si-¹⁹F), the ¹⁸F-labelled/not radioactive chelate of a therapeutic tracer can be used for pretherapeutic dosimetry by means of PET imaging by using a tracer with exactly the same chemical structure and thus in vivo properties.

In addition, the combination of SiFA, chelator and additional hydrophilic building blocks makes it possible to modulate the pharmacokinetic properties, suited best for the imaging or therapy of SST expressing tumors. The compounds of the invention are thus particularly suitable for medical applications such as preclinical and clinical imaging, therapeutic applications, such as endoradiotherapy as well as pretherapeutic dosimetry.

In particular, the invention provides a compound of formula (I) or a salt thereof:

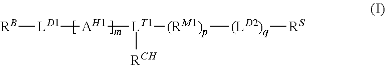

wherein:
R^{B} is a binding motif which is able to bind to a somatostatin receptor;
L^{D1} is a divalent linking group;
m is an integer of 2 to 6, preferably 2 to 5, more preferably 2 or 3;
A^{A1} is, independently for each occurrence, an amino acid unit derived from a hydrophilic amino acid which comprises, in addition to its —NH₂ and its —COOH functional group, a further hydrophilic functional group,
and one of the m units A^{A1} may further carry a hydrophilic unit other than an amino acid bound to its hydrophilic functional group;
L^{T1} is a trivalent linking group;
R^{CH} is a chelating group which optionally contains a chelated radioactive or non-radioactive cation;
R^{M1} is a hydrophilic modifying group, and p is 0 or 1;
L^{D2} is a divalent linking group, and q is 0 or 1; and
R^{S} is a silicon-fluoride acceptor (SiFA) group of one of the following formulae, which comprises a silicon atom and a fluorine atom, and which can be labeled with ¹⁸F by isotopic exchange of ¹⁹F by ¹⁸F or which is labeled with ¹³F:
if p is 1, R^{S} is a group of formula (S-3) or a group of formula (S-4):

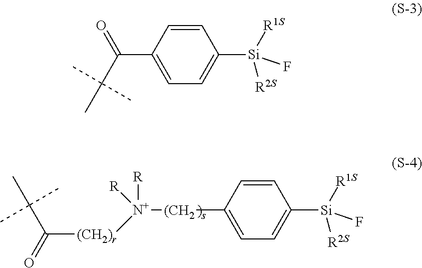

wherein
r is 1, 2 or 3, preferably 1, s in —(CH₂)S— is an integer of 1 to 6 and is preferably 1,
the groups R are, independently, H or C1 to C6 alkyl, preferably H or C1 to C2 alkyl, and are more preferably both methyl, and
R^{1S} and R^{2S} are independently from each other a linear or branched C3 to C10 alkyl group, preferably R^{1S} and R^{2S} are selected from isopropyl and tert-butyl, and more preferably R^{1S} and R^{2S} are tert-butyl; and wherein the dashed line marks a bond which attaches the group to the remainder of the compound; and
if p is 0, R^{S} is a group of formula (S-4) as defined above.

As explained above, the compounds of the invention encompass compounds of formula (I). Moreover, salts, typically pharmaceutically acceptable salts, of the compounds of formula (I) are encompassed by the present invention. Thus, unless indicated to the contrary, any reference to a compound of the invention herein encompasses the compounds of formula (I) (and the preferred embodiments of these formulae disclosed herein), and the salts thereof. Likewise, any racemates, enantiomers, or diastereomers of any chiral compounds of formula (I) and their salts are encompassed, unless a specific stereochemistry of the compound under consideration is indicated in a specific context. Due to their capability of binding to a somatostatin (SST) receptor and of acting as a ligand for such a receptor, the compounds of the invention may also be referred to as SST receptor ligand compounds of the invention, or briefly as ligand compounds of the invention.

In the following a further description will be provided of the compounds of formula (I) and their salts, and of preferred embodiments thereof.

In order to be able to act as somatostatin (SST) receptor binding ligand compounds, the compounds of the invention comprise a binding motif R^{B} which ensures the ligand/receptor interaction to take place between the compounds in accordance with the invention and an SST receptor and thus serves as a fundamental affinity anchor for the compounds towards the SST receptor. R^{B} may also be referred to as a targeting group or a targeting motif in the compounds of the invention. Preferably, R^{B} is able to bind to at least somatostatin receptor 2, or SST₂, or more somatostatin receptor subtypes, or even to all somatostatin receptor subtypes, the latter resulting in so called SST pan-receptor ligands.

The binding motif R^{B} is capable of binding with high affinity to one or more SST receptors. In this context, high affinity binding preferably means that the ligand compound comprising the binding motif exhibits an IC50 in the low nanomolar range, preferably 50 nM or less, more preferably 10 nM or less, still more preferably 5 nM or less. For the sake of clarity, the half maximal inhibitory concentration (IC50) is defined here as the quantitative measure of the molar concentration of binding motif R^{B} or a ligand compound according to formula (I) or (II) necessary to inhibit the binding of a radioactive reference ligand, here [¹²¹]Tyr³-Octreotide, in vitro to SST receptors by 50%. [1]

It will be understood that a preferred binding motif which is capable of high affinity binding to an SST receptor as referred to herein may show high affinity to more than one SST receptor type. Preferably, the binding motif R^{B} is one which shows the highest binding affinity among SST receptor subtypes to SST₂.

Suitable binding motifs include agonists and antagonists of an SST receptor.

Together with a structure which ensures a binding interaction between the compounds in accordance with the invention and an SST receptor, the binding motif R^{B} generally comprises a coupling group, i.e. a functional group which allows R^{B} to be attached to the remainder of the compound of the invention via a covalent bond which is formed between the group R^{B} and L^{D1} or L^{T2}, respectively. The coupling group may consist of one or more atoms. Exemplary coupling groups can be selected from —NH—, —NR— (wherein the group R is C1 to C6 alkyl and is preferably methyl), —C(O)—, —O—, —S—, a quaternary ammonium group, and a thiourea bridge or a group which forms such a thiourea bridge together with a complementary group to which R^{B} is attached. In this context, and also in other instances where reference is made to a quaternary ammonium group as a possible coupling group herein, the quaternary ammonium group is preferably a coupling group of the formula —N(R)₂⁺—, wherein the groups R are independently C1 to C6 alkyl, and are preferably methyl. As will be understood, a coupling group comprised by R^{B} may be covalently linked to a further, complementary coupling group comprised by L^{D1} or L^{T2} in the compound in accordance with the invention, so that the two coupling groups combine to form a binding unit, such as an amide bond (—C(O)—NH—), an alkylated amide bond (—C(O)—NR—), or a thiourea bridge (—NH—C(S)—NH—). As referred to herein, also in further instances below, the substituent R in the alkylated amide bond —C(O)—NR— is C1 to C6 alkyl, preferably methyl. It is preferred that R^{B} comprises a coupling group —NH—, and that the coupling group forms an amide bond —C(O)—NH— with a group —C(O)— provided by L^{D1} or L^{T2}, respectively.

Typically, the binding motif R^{B} comprises a peptide structure, preferably a cyclic peptide structure or a peptide cyclized by a disulfide bridge, capable of binding to an SST. Diverse peptides capable of binding to an SST are known and described in the literature. They can be used to provide the binding motif in a compound of the invention, e.g. by forming an amide bond with the remainder of the compound using a carboxylic acid group or an amino group contained in the peptide.

Thus, the binding motif may comprise a group, and preferably is a group, which can be derived from a receptor agonist or receptor antagonist selected from Tyr³-Octreotate (or Tyr³,Thr⁸-Octreotide, TATE, H-D-Phe-cyclo(L-Cys-L-Tyr-D-Trp-L-Lys-L-Thr-L-Cys)-L-Thr-OH), Thr⁸-Octreotide (ATE), Phe¹,Tyr³-Octreotide (TOC, H-D-Phe-cyclo(L-Cys-L-Tyr-D-Trp-L-Lys-L-Thr-L-Cys)-L-Thr-ol), Nal³-Octreotide (NOC, H-D-Phe-cyclo(L-Cys-L-1-Nal-D-Trp-L-Lys-L-Thr-L-Cys)-L-Thr-ol), 1-Nal³,Thr⁸-Octreotide (NOCATE), BzThi³-Octreotide (BOC), BzThi³,Thr⁸-Octreotide (BOCATE), JR11 (H-L-Cpa-cyclo(D-Cys-L-Aph(Hor)-D-Aph(Cbm)-L-Lys-L-Thr-L-Cys)-D-Tyr-NH₂), BASS (H-L-Phe(4-NO₂)-cyclo(D-Cys-L-Tyr-D-Trp-L-Lys-L-Thr-L-Cys)-D-Tyr-NH₂) and KE121 (cyclo(D-Dab-L-Arg-L-Phe-L-Phe-D-Trp-L-Lys-L-Thr-L-Phe)), more preferably from TATE or JR11, and most preferably from TATE. As will be understood by the skilled reader, the group R^{B} can be conveniently derived from the receptor agonists or antagonists listed above by using a functional group, such as a carboxylic acid group or an amino group, contained in the receptor agonist or antagonist, to provide a coupling group which attaches the group R^{B} to the remainder of the compound. Preferably, these peptidic receptor agonists or receptor antagonists provide the group R^{B} by using an amino group contained therein, e.g. in an optionally substituted phenylalanine unit contained in the peptide, to form an amide bond with the remainder of the compound of the invention. In this case, the covalent bond between R^{B} and R^{D1} (formula (I)), is formed between a terminal —NH— group as a coupling group in R^{B} and a terminal —C(O)— group that may be present as a terminal coupling group in L^{D1}.

Alternatively, as will be understood by the skilled reader, the group R^{B} can be conveniently derived from the receptor agonist or receptor antagonist listed above by the introduction of an additional functional moiety which provides a functional group that allows a chemical bond to be formed with L^{D}, such as a moiety with an isothiocyanate that can link to an amine on L^{D1} to form a thiourea bridge. As will be understood by the skilled reader, other conjugation strategies, typically summarized as “bioconjugation strategies” can also be used to link a group R^{B} in a compound in accordance with the invention to L^{D1}.

In line with the above, the binding motif R^{B} may comprise e.g. a group of the formula (B-1) or of the formula (B-2), and preferably is a group of the formula (B-1) or of the formula (B-2) as shown in the following. Among them, the group of the formula (B-1) if preferred.

The group of the formula (B-1) has the following structure:

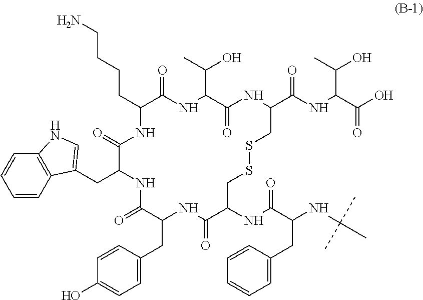

wherein the dashed line marks a bond which attaches the group to the remainder of the compound. As will be understood by the skilled reader, the bond marked by the dashed line in formula (B-1) does not carry a methyl group at its end opposite to the nitrogen atom, but represents a bond which attaches the group R^{B} to the remainder of the compound of formula (I), i.e. in this case to the point of attachment of R^{B} in formula (I). Preferably, the bond marked by the dashed line in formula (B-1) represents a covalent bond which is present in the compounds of the invention between the nitrogen atom of the —NH— group indicated in formula (B-1) and a carbon atom of a carbonyl group which may be present as a terminal group in L^{D1}. Thus, an amide bond is provided.

The group of the formula (B-2) has the following structure:

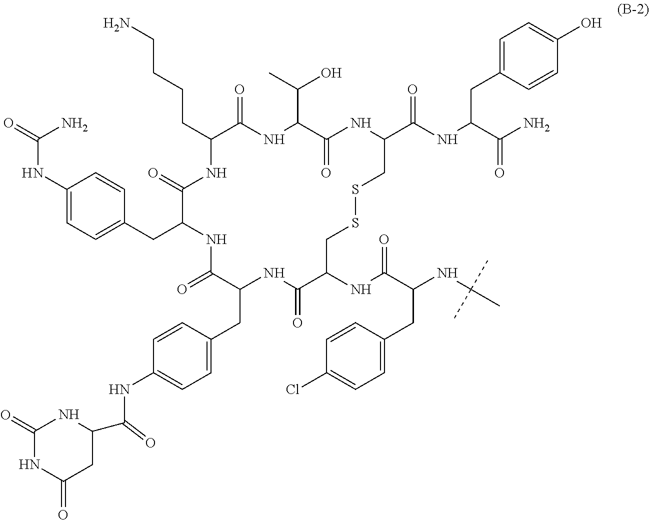

wherein the dashed line marks a bond which attaches the group to the remainder of the compound. As will be understood by the skilled reader, the bond marked by the dashed line in formula (B-2) does not carry a methyl group at its end opposite to the nitrogen atom, but represents a bond which attaches the group R^{B} to the remainder of the compound of formula (I), i.e. in this case to the point of attachment of R^{B} in formula (I). Preferably, the bond marked by the dashed line in formula (B-2) represents a covalent bond which is present in the compounds of the invention between the nitrogen atom of the —NH— group indicated in formula (B-2) and a carbon atom of a carbonyl group which may be present as a terminal group in L^{D1}. Thus, an amide bond is provided.

More preferably, the binding motif R^{B} is a group of the formula (B-1a) or (B-2a), among which the group of the formula (B-1a) is preferred:

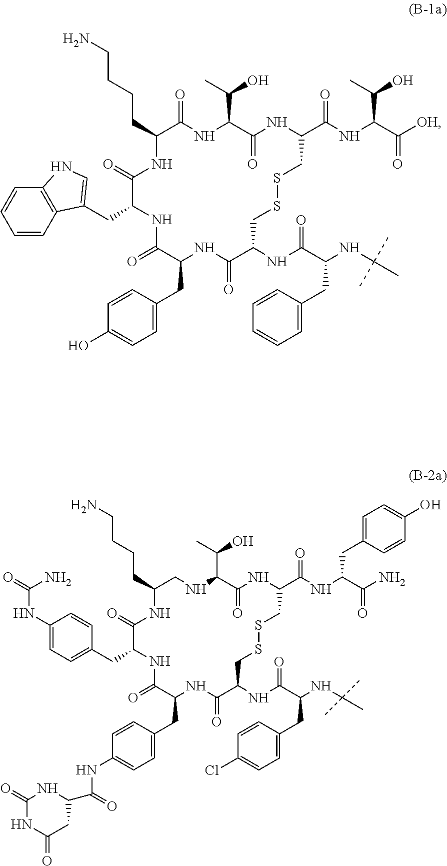

wherein the dashed line marks a bond which attaches the group to the remainder of the compound.

The chelating group R^{CH} in the compounds of formula (I) and their salts is suitable to form a chelate with a radioactive or non-radioactive cation. Diverse chelating agents from which suitable chelating groups can be derived are well known in the art and can be used in the context of the present invention. Metal- or cation-chelating agents, e.g. macrocyclic or acyclic compounds, which are suitable to act as a chelating group, are available from a number of manufacturers. It will be understood that numerous chelating agents can be used in an off-the-shelf manner by a skilled person without further ado. It will further be understood that the suitability of the chelating group to form a chelate with a given cation requires the chelating group to be able to provide a chelated ligand in a chelate complex comprising the cation under consideration, but does not require the chelating group to provide the only ligand of the cation in the chelate complex. Thus, if the chelating group R^{CH} contains a chelated radioactive or non-radioactive cation, the cation may be a complex cation, e.g. a metal ion carrying an additional coordinated ligand other than the chelating group, such as an oxo ligand.

For example, the chelating group R^{CH} may comprise at least one of
(i) a macrocyclic ring structure with 8 to 20 ring atoms of which 2 or more, preferably 3 or more, are selected from oxygen atoms and nitrogen atoms; and
(ii) an acyclic, open chain chelating structure with 8 to 20 main chain atoms of which 2 or more, preferably 3 or more are heteroatoms selected from oxygen atoms and nitrogen atoms.

Preferably, the chelating group R^{CH} is a chelating group which is suitable to form a chelate comprising a cation selected from cations of Sc, Cr, Mn, Co, Fe, Ni, Cu, Ga, Zr, Y, Tc, Ru, Rh, Pd, Ag, In, Sn, Te, Pr, Nd, Gd, Pm, Tb, Sm, Eu, Gd, Tb, Ho, Dy, Er, Yb, Tm, Lu, Re, W, Pt, Ir, Hg, Au, Pb, At, Bi, Ra, Ac, and Th, or a chelate comprising a cationic molecule comprising ¹⁸F or ¹⁸F, such as ¹⁸F−[AlF]²⁺. More preferably, the chelating group is suitable to form a chelate comprising a cation selected from a cation of Cu, Ga, Lu and Pb, and still more preferably a cation of Ga or Lu.

Together with a structure which is suitable as a chelating ligand for a cation, the chelating group R^{CH} generally comprises a coupling group which allows R^{C}H to be attached to the remainder of the compound of the invention via a covalent bond which is formed between the group R^{CH} and L^{T1}. The coupling group may consist of one or more atoms. An exemplary coupling group can be selected from —NH—, —NR— (wherein the group R is C1 to C6 alkyl and is preferably methyl), —C(O)—, —S—, —O—, a quaternary ammonium group, and a thiourea bridge or a group which forms such a thiourea bridge together with a complementary group to which R^{CH} is attached. The coupling group may be covalently linked to a further, complementary coupling group comprised in L^{T1} in the compound of the invention, so that the two coupling groups combine to form a binding unit, such as an amide bond —C(O)—NH—, an alkylated amide bond —C(O)—NR—, or a thiourea bridge. It is preferred that R^{CH} comprises a coupling group —C(O)—, and that the coupling group forms an amide bond —C(O)—NH— with a group —NH— provided by L^{T1}.

The chelating group R^{CH} may comprise a group, preferably is a group, which can be derived from a chelating agent selected from diethylenetriaminepentamethylenephosphonic acid (EDTMP) and its derivatives, diethylenetriaminepentaacetic acid (DTPA) and its derivatives, bis(carboxymethyl)-1,4,8,11-tetraaza-bicyclo[6.6.2] hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino],pentyl]-N-[5-[[4-[5-aminopentyl-(hydroxy)amino]-4-oxobutanoyl]-amino]pentyl]-N-hydroxybutandiamide (DFO) and derivatives thereof, 1,4,7,10-tetraazacyclododecane-1,7-diacetic acid (DO2A), 1,4,7,10-tetraazacyclododecan-N,N′,N″,N′″-tetraacetic acid (DOTA), 2-[1,4,7,10-tetraazacyclododecane-4,7,10-triacetic acid]-pentanedioic acid (DOTAGA or DOTA-GA), 1,4,7,10-tetrakis(carbamoylmethyl)-1,4,7,10-tetraazacyclododecane (DOTAM), N,N′-dipyridoxylethylendiamine-N,N′-diacetate-5,5′-bis(phosphat) (DPDP), diethylenetriaminepentaacetic acid (DTPA), ethylenediamine-N,N′-tetraacetic acid (EDTA), ethyleneglykol-O,O-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclodecan-4,7,10-triacetate (HP-DOA3), 6-hydrazinyl-N-methylpyridine-3-carboxamide (HYNIC), 1,4,7-triazacyclononan-1-succinic acid-4,7-diacetic acid (NODASA), 1-(1-carboxy-3-carboxypropyl)-4,7-(carboxy)-1,4,7-triazacyclononane (NODAGA), 1,4,7-triazacyclononanetriacetic acid (NOTA), 4,11-bis(carboxymethyl)-1,4,8,11-tetraazabicyclo[6.6.2]hexadecane (TE2A), 1,4,8,11-tetraazacyclododecane-1,4,8,11-tetraacetic acid (TETA), terpyridine-bis(methyleneamine) tetraacetic acid (TMT), 1,4,7,10-tetraazacyclotridecan-N,N′,N″,N′″-tetraacetic acid (TRITA), and triethylenetetraaminehexaacetic acid (TTHA), N,N′-bis[(6-carboxy-2-pyridil)methyl]-4,13-diaza-18-crown-6 (H₂macropa), 4-amino-4-{2-[(3-hydroxy-1,6-dimethyl-4-oxo-1,4-dihydro-pyridin-2-ylmethyl)-carbamoyl]-ethyl} heptanedioic acid bis-[(3-hydroxy-1,6-dimethyl-4-oxo-1,4-dihydro-pyridin-2-ylmethyl)-amide] (THP), 1,4,7-triazacyclononane-1,4,7-tris[methylene(2-carboxyethyl)phosphinic acid (TRAP), 2-(4,7,10-tris(2-amino-2-oxoethyl)-1,4,7,10-tetraazacyclododecan-1-yl)acetic acid (DO3AM), and 1,4,7,10-tetraazacyclododecane-1,4,7,10-tetrakis[methylene(2-carboxyethylphosphinic acid)](DOTPI), S-2-(4-isothiocyanatobenzyl)-1,4,7,10-tetraazacyclododecane tetraacetic acid, hydrazinonicotinic acid (HYNIC), 6-amino-6-methylperhydro-1,4-diazepine-N,N,N′,N′-tetraacetic acid (AAZTA) and derivatives thereof, such as (6-pentanoic acid)-6-(amino)methyl-1,4-diazepine triacetate (DATA), pentadeca-1,4,7,10,13-penta-aminopentaacetic acid (PEPA), hexadeca-1,4,7,10,13,16-hexaamine-hexaacetic acid (HEHR), 4-{[bis(phosphonomethyl)) carbamoyl] methyl)-7,10-bis(carboxymethyl)-1,4,7,10-tetraazacyclododec-1-yl) acetic acid (BPAMD), N-(4-{[bis (phosphonomethyl)) carbamoyl] methyl}-7,10-bis (carboxymethyl)-nona-1,4,7-triamine triacetic acid (BPAM), 1,2-[{6-(carboxylate) pyridin-2-yl} methylamine] ethane (DEDPA, H₂DEDPA), deferoxamine (DFO) and its derivatives, deferiprone, (4-acetylamino-4-yl) {2-[(3-hydroxy-1,6-dimethyl-4-oxo-1,4-dihydro-pyridin-2-ylmethyl)-carbamoyl]-ethyl}-heptanedioic acid bis-[(3-hydroxy-1,6-dimethyl-4-oxo-1,4-dihydro-pyridin-2-ylmethyl)-amide](CP256) and its derivatives such as YM103; tetraazycyclodecane-phosphinic acid (TEAP), 6-amino-6-methylperhydro-1,4-diazepine-N,N,N′,N′-tetraacetic acid (AAZTA); 1-N-(4-aminobenzyl)-3,6,10,13,16,19-hexaazabicyclo [6.6.6]-eicosane-1,8-diamine (SarAr), 6,6′-[{9-hydroxy-1,5-bis-(methoxycarbonyl)-2,4-di(pyridin-2-yl)-3,7-diazabicyclo[3.3.1]nonane-3,7-diyl}bis(methylene)]dipicolinic acid (H₂bispa²), 1,2-[{6-(carboxylato)pyridin-2-yl}methylamino]-ethane (H₂dedpa), N,N′-bis(6-carboxy-2-pyridylmethyl)-ethylenediamine-N,N′-diacetic acid (H₄octapa), N,N′-bis(2-hydroxy-5-sulfonylbenzyl)-N,N′-bis-(2-methylpyridyl)ethylenediamine (HeSbbpen) and derivatives thereof, triethylenetetramine-N,N,N′,N″,N′″,N′″-hexaacetic (TTHA), 2-aminomethylpiperidine triacetic acid (2-AMPTA) and derivatives thereof, such as 2-(N-(2-Hydroxybenzyl)aminomethyl)piperidine (2-AMPTA-HB) further functionalized derivative of 2-AMPTA with additional functional groups suitable for conjugation to peptidic structures, 4-nitro-2-hydroxybenzyl-2-{[(6)-trans-2-[benzyl(carboxymethyl)amino]cyclohexyl](carboxymethyl)amino}acetic acid (RESCA) and derivatives thereof, and 6-carboxy-1,4,8,11-tetraazaundecane (N₄) and derivatives thereof. Among these chelating agents, preference is given to CBTE2a, DOTA, DOTAGA, DOTAM, NODASA, NODAGA, NOTA, TE2A, DO3AM, SarAr, 2-AMPTA, 2-AMPTA-HB and RESCA. More preferred are DOTA, DOTAGA, DOTAM, DO3AM, NOTA and NODAGA. Still more preferred are DOTA, DOTAGA and DOTAM. In line with the general definition provided above, the chelating group derived from the exemplary chelating agents listed above optionally contains a chelated radioactive or non-radioactive cation.

As will be understood by the skilled reader, a chelating group in a compound in accordance with the invention can be conveniently derived from the chelating agents listed above by either using a functional group contained in the chelating agent, such as a carboxylic acid group, an amide group, an amino group, a hydroxy group, or a thiol function to provide a coupling group, e.g. selected from —C(O)—, —NH—, —S— and —O—, which attaches the chelating group to the remainder of the compound. Preferably, a carboxylic acid group is used to provide a coupling group —C(O)— (a carbonyl) to form an amide. The coupling group provided by R^{CH} may be covalently linked to a further, complementary coupling group comprised in L^{T1}, respectively, so that the two coupling groups combine to form a binding unit, such as an amide bond —C(O)— NH—. As noted above, it is preferred that R^{CH} comprises a coupling group —C(O)—, and that the coupling group forms an amide bond with a group —NH— provided by L^{T1}.

Alternatively, as will be understood by the skilled reader, a chelating group in a compound in accordance with the invention can be conveniently derived from the chelating agents listed above by the introduction of an additional functional group or the introduction of additional groups having a functional group able to form a chemical bond to L^{T1}, such as a chelator modified with an additional residue with an isothiocyanate that can link to an amine on L^{T1} by means of a thiourea bridge. As will be understood by the skilled reader, other conjugation strategies, typically summarized as “bioconjugation strategies” can also be used to link a chelating group in a compound in accordance with the invention to L^{T1}.

It is particularly preferred that R^{CH} is a group of the formula (CH-1), (CH-2) or (CH-3), or a chelate formed by a group of the formula (CH-1), (CH-2) or (CH-3) and a chelated radioactive or non-radioactive cation, i.e. the group of the formula (CH-1), (CH-2) or (CH-3) optionally contains a chelated radioactive or non-radioactive cation:

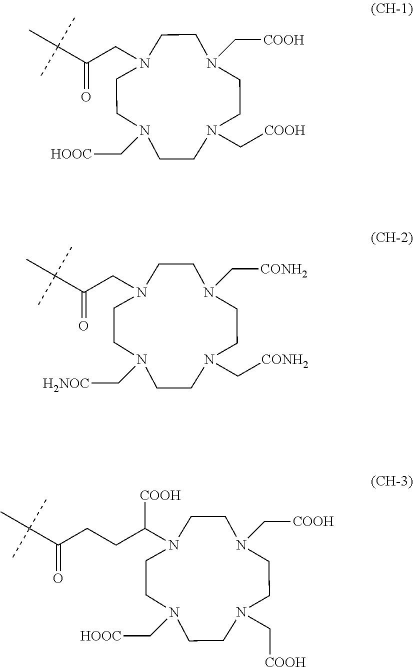

wherein the dashed line marks a bond which attaches the group to the remainder of the compound. As will be understood by the skilled reader, the bond at the carbonyl group marked by the dashed line in formulae (CH-1) to (CH-3) thus does not carry a methyl group at its end opposite to the carbonyl group, but represents a bond which attaches the group R^{CH} to the remainder of the compound of formula (I), i.e. in this case to the point of attachment of R^{C}H in formula (I). Preferably, the bond marked by the dashed line in formulae (CH-1) to (CH-3) represents a covalent bond which is present in the compounds of the invention between the carbon atom of the carbonyl group indicated in in formulae (CH-1) to (CH-3) and a nitrogen atom of an NH group which may be present as a terminal group in L^{T1}. Thus, an amide bond is provided.

The chelated radioactive or non-radioactive cation that may be contained in the chelating group R^{CH} preferably comprises or consists of a cation selected from cations of ⁴³Sc, ⁴⁴Sc, ⁴⁷Sc, ⁵¹Cr, ^{52m}Mn, ⁵⁵Co, ⁵⁷Co, ⁵⁸Co, ⁵²Fe, ⁵⁶Ni, ⁵⁷Ni, ⁶²Cu, ⁶⁴Cu, ⁶⁷Cu, ⁶⁶Ga, ⁶⁸Ga, ⁶⁷Ga, ⁸⁹Zr, ⁹⁰Y, ⁸⁶Y, ^{94m}Tc, ^{99m}Tc, ⁹⁷Ru, ¹⁰⁵Rh, ¹⁰⁹Pd, ¹¹¹Ag, ^{110m}In, ¹¹¹In, ^{113m}In, ^{114m}In, ^{117m}Sn, ¹²¹Sn, ¹²⁷Te, ¹⁴²Pr, ¹⁴³Pr, ¹⁴⁷Nd, ¹⁴⁹Gd, ¹⁴⁹Pm, ¹⁵¹Pm, ¹⁴⁹Tb, ¹⁵²Tb, ¹⁵⁵Tb, ¹⁵³Sm, ¹⁵⁶Eu, ¹⁵⁷Gd, ¹⁵⁵Tb, ¹⁶¹Tb, ¹⁶⁴Tb, ¹⁶¹Ho, ¹⁶⁶Ho, ¹⁵⁷Dy, ¹⁶⁵Dy ¹⁶⁶Dy, ¹⁶⁰Er, ¹⁶⁵Er, ¹⁶⁹Er, ¹⁷¹Er, ¹⁶⁶Yb, ¹⁶⁹Yb, ¹⁷⁵Yb, ¹⁶⁷Tm, ¹⁷²Tm, ¹⁷⁷Lu, ¹⁸⁶Re, ^{186g}Re, ¹⁸⁸Re, ¹⁸⁸W, ¹⁹¹Pt, ^{195m}Pt, ¹⁹⁴Ir, ¹⁹⁷Hg, ¹⁹⁸Au, ¹⁹⁹Au, ²¹²Pb, ²⁰³Pb, ²¹¹At, ²¹²Bi, ²¹³Bi, ²²³Ra, ²²⁴Ra, ²²⁵Ac, ²²⁶Th and ²²⁷Th, and from nonradioactive isotopes of any of these metals, or is a cationic molecule comprising ¹⁸F or ¹⁸F, such as ¹⁸F—[AlF]²⁺. The chelated cation may be a complex cation, e.g. a metal ion carrying an additional coordinated ligand other than the chelating group, such as an oxo-ligand in a chelate including a ^{99m}Tc(V)-oxo core.

Particularly preferred as a chelated cation is a radioactive or non-radioactive cation of Ga, Lu or Pb, such as ¹⁷⁷Lu or ⁶⁸Ga.

Moreover, the compounds in accordance with the invention comprise a silicon-fluoride acceptor (SiFA) group R^{S} which comprises a silicon atom and a fluorine atom and which can be labeled with ¹⁸F by isotopic exchange of ¹⁹F by ¹⁸F or which is labeled with ¹⁸F.

If the variable p in formula (I) is 1, i.e. if the compound of formula (I) or its salt comprises a group R^{M1}, R^{S} is a group of formula (S-3) or a group of formula (S-4):

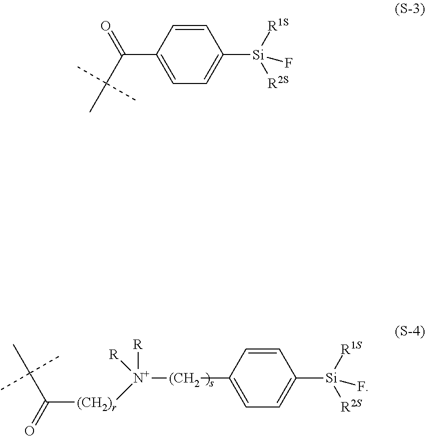

In the group of formula (S-3), R^{1S} and R^{2S} are independently from each other a linear or branched C3 to C10 alkyl group, preferably R^{1S} and R^{2S} are selected from isopropyl and tert-butyl, and more preferably R^{1S} and R^{2S} are tert-butyl. The dashed line marks a bond which attaches the group to the remainder of the compound.

In the group of formula (S-4), r is 1, 2 or 3, preferably 1, s in —(CH₂)ₛ— is an integer of 1 to 6 and is preferably 1,
the groups R are, independently, H or C1 to C6 alkyl, preferably H or C1 to C2 alkyl, and are more preferably both methyl, and
R^{1S} and R^{2S} are independently from each other a linear or branched C3 to C10 alkyl group, preferably R^{1S} and R^{2S} are selected from isopropyl and tert-butyl, and more preferably R^{1S} and R^{2S} are tert-butyl. The dashed line marks a bond which attaches the group to the remainder of the compound.

If the variable p in formula (I) is 0, i.e. if the compound of formula (I) or its salt does not comprise a group R^{M1}, R^{S} is a group of formula (S-4) as defined above.

Exemplary counterions for the positively charged quaternary ammonium group indicated in formula (S-4) which carries two substituents R are anions as they are discussed herein with regard to salts forms of the compound of formula (I), which include, e.g., trifluoro acetate anions or acetate anions.

As will be understood by the skilled reader, the bond marked by the dashed line in formulae (S-3) and (S-4) does not carry a methyl group at its end opposite to the carbonyl group, but represents a bond which attaches the SiFA group to the remainder of the compound of formula (I), i.e. in this case to the point of attachment of R^{S} in formula (I). Preferably, the bond marked by the dashed line in formulae (S-3) and (S-4) represents a covalent bond which is present in the compounds of the invention between the carbon atom of the carbonyl group indicated in formulae (S-3) and (S-4) and a nitrogen atom of an —NH group which may be present as a terminal group in the unit carrying R^{S} (i.e. L^{T1}, R^{M1} or L^{D2} in formula (I)). Thus, an amide bond is provided.

The fluorine atom indicated in formulae (S-3) and (S-4) may be a ¹⁸F atom, or a ¹⁹F atom which can be exchanged to provide ¹⁸F by isotopic exchange of ¹⁹F by ¹⁸F.

Among the groups (S-3) and (S-4), most preferred as a group R^{S} is the group (S-4).

Furthermore, the group (S-3) is preferably a group (S-3a), and the group (S-4) is preferably a group (S-4a):

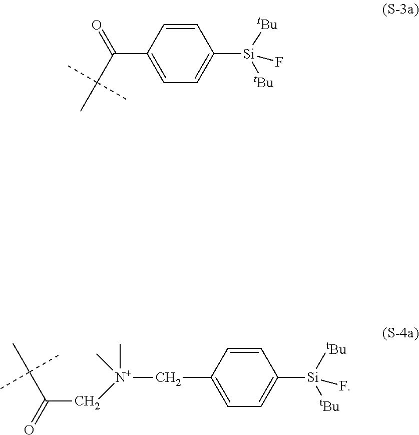

wherein ^{t}Bu indicates a tert-butyl group and the dashed line marks a bond which attaches the group to the remainder of the compound. As noted above with respect to (S-4), exemplary counterions for the positively charged quaternary ammonium group indicated in formula (S-4a) which carries two methyl substituents are anions as they are discussed herein with regard to salts forms of the compound of formula (I), which include, e.g., trifluoro acetate anions or acetate anions.

It will be understood that the above explanations regarding the SiFA group of formulae (S-3) and (S-3a) and (S-4) and (S-4a), respectively, not only apply for formula (I), but also for the preferred embodiments thereof, wherein the SiFA groups R^{S1}, R^{S2} or R^{S3} define a group of formula (S-3) and preferably (S-3a), a group formula (S-4) and preferably (S-4a), or both.

The scaffold structure carrying the groups R^{B}, R^{CH} and R^{S} in formula (I) as shown below

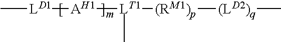

comprises one or more amino acid units. As will be understood by the skilled person, an amino acid unit is a group which can be derived from an amino acid, i.e. from a compound comprising an amino group and a carboxylic acid group in the same molecule. Unless indicated otherwise in a specific context, one or more further functional groups in addition to the amino group and the carboxylic acid group may be present in the amino acid from which the amino acid unit can be derived. A specific amino acid unit is typically identified by the name of the amino acid from which it can be derived, e.g. as a glycine unit, asparagine unit, etc. Unless indicated otherwise in a specific context, the amino acids from which the amino acid units can be derived are preferably α-amino acids. If an amino acid unit comprised in the scaffold structures can be derived from a chiral amino acid, preference is given to the D-configuration.

As will be further understood, an amino acid unit can be derived from an amino acid by using one or more of its functional groups to provide a coupling group which forms a bond to an adjacent atom or group to which the amino acid unit is attached. For example, an amino group of the amino acid may be used to provide a coupling group —NH— wherein the bond to one hydrogen atom is replaced by a bond to another adjacent atom or group. A carboxylic acid group of the amino acid may be used to provide a coupling group —C(O)— wherein the bond to the —OH group is replaced by a bond to another adjacent atom or group. Preferably, any coupling group provided by the amino acid is covalently linked to a further, complementary coupling group in the compound in accordance with the invention, so that the two complementary coupling groups combine to form a binding unit, such as an amide bond (—C(O)— NH—) or an alkylated amide bond —C(O)—NR—, preferably an amide bond. R is C1 to C6 alkyl, preferably methyl. If the amino acid providing the amino acid unit comprises a further functional group which is different from an amino or a carboxylic acid group, a coupling group different from a —NH— and a —C(O)— coupling group may be provided, e.g. —O— or —S—.

If the amino acid unit is a monovalent unit, it is preferred that the unit is attached in the compound of the invention with one amide bond formed using either an amino group or a carboxylic acid group provided by the amino acid from which the amino acid unit is derived. If the amino acid unit is a divalent unit, it is preferred that the unit is attached in the compound of the invention with two amide bonds formed using an amino group and a carboxylic acid group provided by the amino acid from which the amino acid unit is derived. If the amino acid unit is a trivalent unit, as it can be provided by an amino acid comprising a further functional group in addition to the amino group and the carboxylic acid group required for an amino acid, it is preferred that the further functional group is also an amino or a carboxylic acid group, and that the unit is attached in the compound of the invention with three amide bonds formed using an amino group, a carboxylic acid group and the further functional group provided by the amino acid from which the amino acid unit is derived.

In the following, the structural elements of the scaffold structure carrying R^{B}, R^{CH} and R^{S} in formula (I)

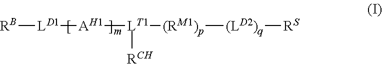

shall be further discussed.

The divalent linking group L^{D1} provides a link between the group R^{B} and the amino acid units A^{H1}. Thus, L^{D1} typically contains a coupling group at its terminus to which R^{B} is attached which is suitable to form a binding unit, such as an amide bond (—C(O)—NH—), an alkylated amide bond —C(O)—NR—, or a thiourea bridge, preferably an amide bond, with a complementary group contained in R^{B}. Reference is made to the discussion of coupling groups in the context of the definition of R^{B} above. Preferably, this coupling group in L^{D1} is a group —C(O)—. Likewise, L^{D1} typically contains a coupling group at its terminus to which the amino acid units A^{H1} are attached which is suitable to form a binding unit, such as an amide bond or an alkylated amide bond, preferably an amide bond, with a complementary group contained in A^{H1}. Preferably, this coupling group in L^{D1} is a group —NH—.

In one preferred embodiment, L^{D1} comprises, typically in addition to the coupling groups referred to above, a divalent oligo- or polyethylene glycol group; preferably a divalent oligo- or polyethylene glycol group having 10 or less ethylene glycol units, and more preferably a divalent oligo- or polyethylene glycol group having 2 to 5 ethylene glycol units.

In accordance with this embodiment, L^{D1} can be a divalent group of formula (L-1a):

—C(O)—(CH₂)ₐ—(O—CH₂—CH₂)_{b}—NH— (L-1a)

wherein
a is 1 or 2, preferably 1; and
b is an integer of 2 to 5, preferably 2 or 3.

The bond at the C-terminus of the above formula is preferably formed with R^{D}.

In another, more preferred embodiment, the divalent linking group L^{D1} comprises a divalent amino acid unit or a divalent chain of amino acid units, more preferably a divalent chain of 2 to 5 amino acid units, still more preferably a divalent chain of 2 or 3 amino acid units. Due to the functional groups contained in an amino acid, the amino acid unit/chain of amino acid can also provide the coupling groups discussed above for attachment of R^{B} or A^{H1}. Thus, the divalent linking group L^{D1} can consist of a divalent amino acid unit or a divalent chain of amino acid units, more preferably a divalent chain of 2 to 5 amino acid units, still more preferably a divalent chain of 2 or 3 amino acid units.

In accordance with this embodiment, L^{D1} can be represented by formula (L-1b):

-[A^{L1}]ₙ- (L-1b)

wherein
A^{L1} is, independently for each occurrence if n is more than 1, an amino acid unit; and
n is an integer of 1 to 5, preferably 2 to 5, more preferably 2 or 3.

Preferably, the group (L-1b) provides a C-terminus which forms a bond with R^{B}, and an N-terminus which forms a bond with A^{H1}

If L^{D1} comprises or consists of one or more amino acid units, it is preferred that these amino acid units do not contain any free amino groups, free acid groups, or salts thereof. It is more preferred that the amino acid units do not contain any functional group which carries a charge at a pH of 7.0. For example, an amino acid unit comprised by L^{D1} can be selected, independently, from an amino acid unit provided by glycine, ß-alanine, or γ-aminobutyric acid and from an amino acid unit comprising a side chain selected from a C1-C4 alkyl group such as methyl, —(CH₂) NH—C(═NH)—NH₂, —(CH₂)ᵥ—C(═O)NH₂, —(CH₂)ᵥ—NH—C(═O)—NH₂, and —(CH₂)ᵥ—OH, wherein v is 1 to 4, e.g. 1 or 2.

If L^{D1} comprises or consists of one or more amino acid units, it is more preferred that each amino acid unit in L^{D1} is selected, independently for each occurrence if more than one amino acid unit is present in L^{D1}, from a glycine (Gly) unit, ß-alanine unit, alanine (Ala) unit, asparagine (Asn) unit, glutamine (Gln) unit, and a citrulline (Cit) unit. Still more preferred are amino acid units selected from a glycine unit and an asparagine unit. With respect to their stereochemistry, the amino acid units, except for the glycine unit, are preferably D-amino acid units. Thus, for example, a preferred group -[A^{L1}]ₙ- may consist of (i) one Gly unit, (ii) two Gly units, (iii) three Gly units, or (iv) two D-Asn units, and a particularly preferred example is a group -[A^{L1}]ₙ- consisting of two or three Gly units.

The variable m is an integer of 2 to 6, preferably 2 to 5, more preferably 2 or 3.

A^{H1} is, independently for each occurrence, an amino acid unit derived from a hydrophilic amino acid which comprises, in addition to its —NH₂ and its —COOH functional group, a further hydrophilic functional group. Such a unit may be briefly referred to herein as “hydrophilic amino acid unit”. One of the m units A^{A1} may optionally further carry a hydrophilic unit other than an amino acid bound to its hydrophilic functional group.

For example, the further hydrophilic functional group of the amino acid units A^{A1} can be selected, independently for each occurrence, from —NH₂, —COOH, —NH—C(═NH)—NH₂, —C(═O)NH₂, —NH—C(═O)—NH₂, —OH and —P(═O)(OH)₂. Among these, preferred are —NH₂, —COOH, —NH—C(═NH)—NH₂, —C(═O)NH₂, and —NH—C(═O)—NH₂.

Preferably, each of the m amino acid unit(s) A^{A1} comprises, independently for each occurrence if m is more than 1, a side chain having a terminal hydrophilic functional group which side chain is selected from —(CH₂)ᵥ—NH₂, —(CH₂)ᵥ COOH, —(CH₂)ᵥ—NH—C(═NH)—NH₂, —(CH₂)ᵥC(═O)NH₂, —(CH₂)ᵥ—NH—C(═O)—NH₂, —(CH₂)—OH and —(CH₂)ᵥ—P(═O)(OH)₂ wherein
v is 1 to 4,

and wherein the terminal functional group of the above side chain in one of the m units A^{H1} may form a bond with an additional hydrophilic unit other than an amino acid unit. In line with the above, the side chain is more preferably selected from —(CH₂)ᵥ—NH₂, —(CH₂)ᵥ—COOH, —(CH₂)˜—NH—C(═NH)—NH₂, —(CH₂)ᵥ—C(═O)NH₂, and —(CH₂)ᵥ—NH—C(═O)—NH₂, wherein v is 1 to 4. As will be understood by the skilled reader, the side chain of the amino acid unit A^{A1} is not involved in a bond to L^{D}, L^{T1} or to an adjacent unit A^{H1}. In line with the above, one of the m units A^{H1} may optionally further carry a hydrophilic unit other than an amino acid bound to the terminal hydrophilic functional group of the side chain.

Thus, it is preferred that the amino acid unit(s) A^{H1} is (are) selected, independently for each occurrence, from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, serine (Ser) unit, citrulline (Cit) unit, thiocitrullin unit, methylisothiocitrulline unit, canavanin unit, thiocanavanin unit, α-amino-γ-(thioureaoxy)-n-butyric acid unit, α-amino-γ-(thioureathia)-n-butyric acid unit, and a phosphonomethylalanine (Pma) unit. They are preferably units which can be derived from amino acids in D-configuration. More preferred are units selected from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, serine (Ser) unit, a citrulline (Cit) unit and a phosphonomethylalanine (Pma) unit. Still more preferred are units selected from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, and a citrulline (Cit) unit. Thus, in line with the above, it is particularly preferred if the group [A^{H1}]ₘ consists of 2 or 3 amino acid units independently selected from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, and a glutamine (Gln) unit. For example, a preferred group [A^{H1}]ₘ may consist of (i) two D-Asp units, (ii) two D-Orn units, (iii) two D-Asn units, or (iv) two D-Asp units and a third unit selected from D-Lys, D-Cit and D-Glu.

One of the units A^{A1} which are selected accordingly may optionally further carry a hydrophilic unit other than an amino acid bound to the terminal hydrophilic functional group that is provided in these amino acid units. The optional hydrophilic unit other than an amino acid unit which is optionally bound to one of the units A^{A1} can be, for example, a carbohydrate group or a trimesic acid group.

Preferably, the group -[A^{H1}]ₘ- forms an amide bond (—C(O)—NH—) or an alkylated amide bond (—C(O)—NR—) with L^{D1}, and an amide bond or an alkylated amide bond with L^{T1}. More preferably, the bonds are amide bonds.

Preferably, the group -[A^{A1}]ₘ- provides a C-terminus which forms a bond with L^{D1}, and an N-terminus which forms a bond with L^{T1}.

The trivalent linking group L^{T1} provides a link between the amino acid unit(s) A^{A1}, R^{CH} and, depending on p and q, R^{M1}, L^{D2} or R^{S}. Thus, L^{T1} typically contains a coupling group at its terminus to which A^{A1} is (are) attached, which is suitable to form a binding unit, such as an amide bond (—C(O)—NH—) or an alkylated amide bond (—C(O)—NR—), preferably an amide bond, with a complementary group contained in A^{A1}. Preferably, this coupling group in L^{T1} is a group —C(O)—. Likewise, L^{T1} typically contains a coupling group, either at its terminus or in a side chain, to which R^{CH} is attached which is suitable to form a binding unit, such as an amide bond, an alkylated amide bond (—C(O)—NR—), or a thiourea bridge, preferably an amide bond, with a complementary group contained in R^{CH}. Reference is made to the discussion of coupling groups in the above definition of R^{CH}. Preferably, this coupling group in L^{T1} is a group —NH—.

At the terminus to which R^{M1}, L^{D2} or R^{S} is attached, L^{T1} typically contains a coupling group selected from the following (i) and (ii), with (i) being preferred.
(i) A coupling group which is suitable to form a binding unit, such as an amide bond, an alkylated amide bond (—C(O)—NR—), or a thiourea bridge, preferably an amide bond, with a complementary group contained in R^{M1}, L^{D2} or R^{S}. Reference is made to the discussion of coupling groups in the above definition of R^{S}. Preferably, this coupling group in L^{T1} is also a group —NH—, which is typically derived from an amino group.
(ii) A quaternary ammonium group as a coupling group. As noted above, the quaternary ammonium group is preferably a coupling group of the formula —N(R)₂⁺—, wherein the groups R are independently C1 to C6 alkyl, and are preferably methyl. This option can be suitably used, e.g., if both p and q are 0 and R^{S} is directly attached to L^{T1}, for example if R^{S} is a group (S-5) as discussed above and provides a substituent attached to the nitrogen atom of the quaternary ammonium group.

It is preferred that L^{T1} is a trivalent amino acid unit. More preferably L^{T1} is a trivalent amino acid unit selected from the following (i) and (ii), with (i) being further preferred.
(i) A trivalent amino acid unit which can be derived from an amino acid comprising, together with the carboxylic acid group and the amino group, a further functional group selected form a carboxylic acid group and an amino group. More preferably, L^{T1} is selected from a 2,3-diaminopropionic acid (Dap) unit, 2,4-diaminobutanoic acid (Dab) unit, ornithine (Orn) unit and a lysine (Lys) unit, most preferably a Dap unit. With respect to their stereochemistry, these amino acid units are preferably D-amino acid units.
(ii) A trivalent amino acid unit comprising a —N(R)₂⁺— group which unit can be derived from a trifunctional amino acid comprising a tertiary amino group as a third functional group in addition to its —NH₂ group and its —COOH group, preferably an amino acid selected from N-dialkylated 2,3-diaminopropionic acid (Dap), N-dialkylated 2,4-diaminobutanoic acid (Dab), N-dialkylated ornithine (Orn) and N-dialkylated lysine (Lys), most preferably an amino acid selected from N-dimethylated 2,3-diaminopropionic acid (Dap), N-dimethylated 2,4-diaminobutanoic acid (Dab), N-dimethylated ornithine (Orn) and N-dimethylated lysine (Lys).

As will be appreciated, the tertiary amino group can be converted to a quaternary ammonium group —N(R)₂⁺—, preferably —N(CH₃)₂⁺— via conjugation with R^{M1}, L^{D2} or R^{S}, preferably R^{S}. With respect to their stereochemistry, these amino acid units are preferably D-amino acid units.

For the trivalent amino acid unit (i), it is preferred that the amino acid unit is attached in the compound of the invention by three amide bonds. Moreover, it is preferred that the amino acid unit is oriented to provide a —C(O)— coupling group attached to A^{H1}, and a —NH— coupling group attached to R^{CH}.

The variable p is 0 or 1, i.e. the hydrophilic modifying group R^{M1} can be present or absent. If it is 0, so that R^{M1} is absent, L^{T1} forms a bond with either L^{D2} (if q is 1) or with R^{S} (if q is also 0, so that L^{D2} is also absent).

The hydrophilic modifying group R^{M1} is a divalent group which comprises a hydrophilic moiety, i.e. typically a polar or charged moiety. Typically, R^{M1} contains a coupling group at its terminus to which L^{T1} is attached which is suitable to from a binding unit, such as an amide bond (—C(O)— NH—) or an alkylated amide bond (—C(O)—NR—), preferably an amide bond, with a complementary group contained in L^{T1}. Preferably, this coupling group in R^{M1} is a group —C(O)—. At the terminus to which L^{D2} or R^{S} is attached, R^{M1} preferably provides a coupling group which is suitable to form a binding unit, such as an amide bond or an alkylated amide bond (—C(O)—NR—), preferably an amide bond, with a complementary group contained in L^{D2} or R^{S}. Preferably, this coupling group in R^{M1} is a group —NH—.

Preferably, the hydrophilic modifying group R^{M1}, if present, is a group that can be derived from a hydrophilic amino acid which comprises, in addition to its —NH₂ and its —COOH functional group, a further hydrophilic functional group, preferably a hydrophilic functional group selected from —NH₂, —COOH, —NH—C(═NH)—NH₂, —C(═O)NH₂, —NH—C(═O)—NH₂, —NH—C(═S)—NH₂, —O—NH—C(═S)—NH₂, —O—NH—C(═N)—NH₂—OH and —P(═O)(OH)₂, among which a —NH₂ group is further preferred.

More preferably, the hydrophilic modifying group R^{M1}, if present, is a divalent amino acid unit which is selected from a diaminopropionic acid (Dap) unit, 2,4-diaminobutanoic acid (Dab) unit, ornithine (Orn) unit and a lysine (Lys) unit, most preferably a diaminopropionic acid unit. With a view to their stereochemistry, these amino acid units are preferably D-amino acid units.

It is preferred that the hydrophilic modifying group forms an amide bond with L^{T1} and an amide bond with L^{D2} if q is 1 or with R^{S} if q is 0.

The variable q is 0 or 1, i.e. the divalent linking group L¹² can be present or absent. If it is 0, so that L° ² is absent, R^{S} forms a bond with either R^{M1} (if p is 1) or with L^{T1} (if p is also 0).

The optional divalent linking group L^{D2} can act as an additional spacer between R^{S} and the remainder of the compound of the invention. Typically, L^{D2} contains a coupling group at its terminus to which L^{T1} or R^{M4} is attached which is suitable to from a binding unit, such as an amide bond (—C(O)—NH—) or an alkylated amide bond (—C(O)—NR—), preferably an amide bond, with a complementary group contained in L^{T1} or R^{M1}. Preferably, this coupling group in L^{D2} is a group —C(O)—. At the terminus to which L^{D2} or R^{S} is attached, L¹² preferably provides a coupling group which is suitable to form a binding unit, such as an amide bond or an alkylated amide bond, preferably an amide bond, with a complementary group contained in L¹² or R^{S}.

Preferably, this coupling group in L^{D2} is a group —NH—.

The divalent linking group L^{D2}, if present, is preferably a group of the formula (L-2):

-[A^{L2}]_{w}- (L-2)

wherein
A^{L2} is, independently for each occurrence if w is more than 1, an amino acid unit; and
w is 1 to 5, preferably 1 to 3, more preferably 1 or 2.

Preferably, the amino acid unit(s) A^{L2} is (are) independently selected from a glycine unit and an alanine unit. The alanine unit is preferably a D-alanine unit.

A preferred type of compound of the invention is one of the formula (I.1) or a salt thereof:

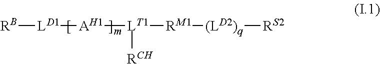

wherein R^{B}, L^{D1}, A^{A1}, m, L^{T1}, R^{C}H, R^{M1}, L^{D2} and q are as defined herein above, including their preferred embodiments, and R^{S2} is a SiFA group of the formula (S-3), more preferably (S-3a) as defined herein above, i.e.:

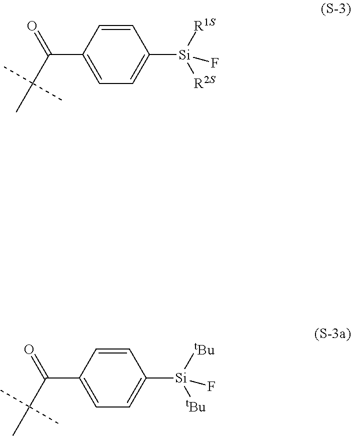

wherein R^{1S} and R^{2S} are as defined herein above, including their preferred embodiments.

Among the compounds of formula (I.1) or their salts, further preference is given to those of formula (I.2) or their salts:

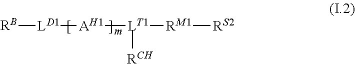

wherein R^{B}, L^{D1}, A^{H1} m L^{T1}, R^{CH}, R^{M1} and R^{S2} are as defined herein above, including their preferred embodiments.

A still more preferred type of compound of the invention is one of the formula (I.3) or a salt thereof:

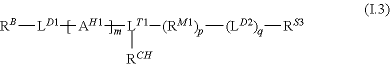

wherein R^{B}, L^{D1}, A^{H1}, m, L^{T1}, R^{CH}, R^{M1}, p, L^{D2} and q are as defined herein above, including their preferred embodiments, and R^{S3} is a SiFA group of the formula (S-4), more preferably (S-4a) as defined herein above, i.e.:

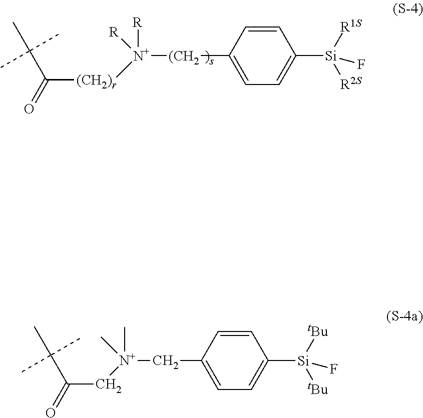

wherein r, s, R, R^{1S} and R^{2S} are as defined herein above, including their preferred embodiments.

Among the compounds of formula (I.3) or their salts, further preference is given to those of formula (I.4) and (I.5) or their salts:

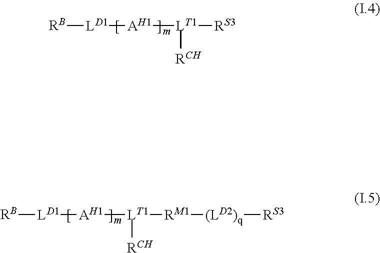

wherein R^{B}, L^{D1}, A^{H1}, m, L^{T1}, R^{CH}, R^{M1}, L^{D2}, q and R^{S3} are as defined herein above, including their preferred embodiments.

In line with the above, it will be understood that it is preferred for the compounds of formula (I) and their salts, as well as for the compounds of formula (I.1) to (I.5) and their respective salts if
R^{B} comprises a group which can be derived from a receptor agonist or receptor antagonist selected from Tyr³, Thr⁸-Octreotide (TATE), Tyr³-Octreotide (TOC), Thr³-Octreotide (ATE); 1-Nal³-Octreotide (NOC), 1-Nal³,Thr⁶-octreotide (NOCATE), BzThi³-octreotide (BOC), BzThi³,Thr⁸-octreotide (BOCATE), JR11, BASS, and KE121;
R^{CH} comprises a group which can be derived from a chelating agent selected from DOTA, DOTAGA, DOTAM, DO3AM, NOTA and NODAGA, and wherein the chelating group optionally comprises a chelated radioactive or non-radioactive cation; and
A^{A1} is selected, independently for each occurrence, from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, serine (Ser) unit, citrulline (Cit) unit and a phosphonomethylalanine (Pma) unit.

It is further preferred for the compounds of formula (I) and their salts, as well as for the compounds of formula (I.1) to (I.5) and their respective salts, if R^{B} comprises a group which can be derived from a receptor agonist or receptor antagonist selected from Tyr³, Thr⁸-Octreotide (TATE), Tyr³-Octreotide (TOC), Thr⁸-Octreotide (ATE); 1-Nal³-Octreotide (NOC), 1-Nal³,Thr⁸-octreotide (NOCATE), BzThi³-octreotide (BOC), BzThi³,Thr⁸-octreotide (BOCATE), JR11, BASS, and KE121;
R^{CH} comprises a group which can be derived from a chelating agent selected from DOTA, DOTAGA, DOTAM, DO3AM, NOTA and NODAGA, and wherein the chelating group optionally comprises a chelated radioactive or non-radioactive cation;
A^{A1} is selected, independently for each occurrence, from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, serine (Ser) unit, citrulline (Cit) unit and a phosphonomethylalanine (Pma) unit; and
R^{M1} if present, is an amino acid unit which is selected from a diaminopropionic acid (Dap) unit, 2,4-diaminobutanoic acid (Dab) unit, ornithine (Orn) unit and a lysine (Lys) unit, and is more preferably a diaminopropionic acid unit.

It is still further preferred for the compounds of formula (I) and their salts, as well as for the compounds of formula (I.1) to (I.5) and their respective salts, if R^{B} comprises a group which can be derived from a receptor agonist or receptor antagonist selected from Tyr³, Thr⁸-Octreotide (TATE), and JR11;
R^{CH} comprises a group which can be derived from a chelating agent selected from DOTA, DOTAGA, and DOTAM, and wherein the chelating group optionally comprises a chelated radioactive or non-radioactive cation;
m is 2 or 3;
A^{H1} is selected, independently for each occurrence, from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, and a citrulline (Cit) unit; and
R^{M1} if present, is an amino acid unit which is selected from a diaminopropionic acid (Dap) unit, 2,4-diaminobutanoic acid (Dab) unit, ornithine (Orn) unit and a lysine (Lys) unit, and is more preferably a diaminopropionic acid unit.

In line with the above discussion of the compounds of formula (I) and their salts, a preferred structure of these compounds is illustrated by formula (Ia) and their salts:

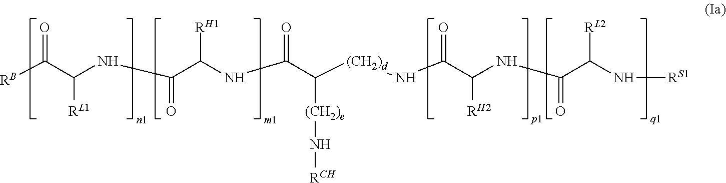

wherein
R^{B} and R^{CH} are as defined herein above, including preferred embodiments thereof;
n1 is 2 to 5, preferably 2 or 3;
R^{L1} is selected, independently for each occurrence from H, a C1-C4 alkyl group, —(CH₂), —NH—C(═NH)—NH₂, —(CH₂)—C(═O)NH₂, —(CH₂)ᵥ—NH—C(═O)—NH₂, and —(CH₂)ᵥ—OH wherein v is 1 to 4;
m1 is 2 to 5, more preferably 2 or 3;
R^{H1} is selected, independently for each occurrence, from —(CH₂)ᵥ—NH₂, —(CH₂)ᵥ—COOH, —(CH₂)ᵥ—NH—C(═NH)—NH₂, —(CH₂)ᵥ—C(═O)NH₂, —(CH₂)ᵥ—NH—C(═O)—NH₂, —(CH₂)ᵥ—OH and —(CH₂)ᵥ—P(═O)(OH)₂, wherein v is 1 to 4;
d is an integer of 0 to 4;
e is an integer of 0 to 4;
and preferably one of d and e is 0 and the other one is an integer of 1 to 4, more preferably the other one is 1;
p1 is 0 or 1;
R^{H2} is selected from —(CH₂)ᵥ—NH₂, —(CH₂)˜—COOH, —(CH₂)ᵥ—NH—C(═NH)—NH₂, —(CH₂)ᵥ—C(═O)NH₂, —(CH₂)ᵥ—NH—C(═O)—NH₂, —(CH₂)ᵥ—OH and —(CH₂)ᵥ—P(═O)(OH)₂ wherein v is 1 to 4;
q1 is an integer of 0 to 2;
R^{L2} is selected from H and CH₃, and
if p1 is 1, R^{S1} is selected from a group of formula (S-3) and a group of formula (S-4)

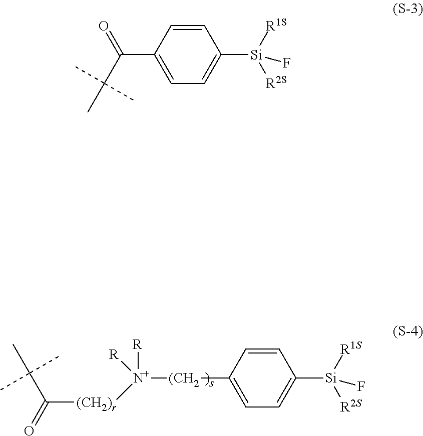

wherein
r is 1, 2 or 3, preferably 1, s is an integer of 1 to 6 and is preferably 1, R is, independently, H or C1 to C6 alkyl, preferably H or C1 to C2 alkyl, and is more preferably methyl, and
R^{1S} and R^{2S} are independently from each other a linear or branched C3 to C10 alkyl group, preferably R^{1S} and R^{2S} are selected from isopropyl and tert-butyl, and more preferably R^{1S} and R^{2S} are tert-butyl; and wherein the dashed line marks a bond which attaches the group to the remainder of the compound; and
if p1 is 0, R^{S1} is a group of formula (S-4) as defined above. It will be understood that the groups of formula (S-3a) and (S-4a) remain even more preferred examples of these.

In formula (Ia), R^{B} and R^{CH} are as defined herein, including preferred embodiments thereof, so that (B-1) or (B-2) remain preferred structures for R^{B}, and (CH-1) to (CH-3) or chelates formed with these chelating groups remain strongly preferred structures for R^{CH} It will be appreciated that the unit —C(O)—CH(R^{L1})—NH— within the brackets [ . . . ]ₙ₁ represents an amino acid unit, and R^{L1} is H or a side chain of the amino acid unit. If the amino acid unit can be derived from a chiral amino acid, it is preferably in D-configuration. R^{L1} is selected, independently for each occurrence from H, a C1-C4 alkyl group such as methyl, —(CH₂)ᵥ—NH—C(═NH)—NH₂, —(CH₂)ᵥ—C(═O)NH₂, —(CH₂)ᵥ—NH—C(═O)—NH₂, and —(CH₂)ᵥ—OH wherein v is 1 to 4, e.g. 1 or 2. Preferably, each of the amino acid units carrying R^{L1} is independently selected from a glycine unit and an asparagine unit, and still more preferably from a glycine unit and a D-asparagine unit. Most preferred is a glycine unit. Thus, for example, a preferred group —[C(O)—CH(R^{L})—NH]ₙr may consist of (i) one Gly unit, (ii) two Gly units, (iii) three Gly units, or (iv) two D-Asn units, and a particularly preferred example is a group —[C(O)—CH(R^{L1})—NH]ₙ₁— consisting of two or three Gly units.

The unit —C(O)—CH(R^{H1})—NH— within the brackets [ . . . ]ₘ₁ likewise represents an amino acid unit, and R^{H1} is a side chain of the amino acid unit. The amino acid from which the amino acid unit is derived is preferably in D-configuration. R^{H1} is selected, independently for each occurrence, from
—(CH₂)ᵥ—NH₂, —(CH₂)ᵥ—COOH, —(CH₂)ᵥ—NH—C(═NH)—NH₂, —(CH₂)˜—C(═O)NH₂, —(CH₂)ᵥ—NH—C(═O)—NH₂, —(CH₂)ᵥ OH and —(CH₂)ᵥ—P(═O)(OH)₂, wherein v is 1 to 4.

R^{H1} is preferably selected from —(CH₂)ᵥ—NH₂, —(CH₂)ᵥ—COOH, —(CH₂)ᵥ—NH—C(═NH)—NH₂, —(CH₂)ᵥ—C(═O)NH₂, and —(CH₂)ᵥ—NH—C(═O)—NH₂, wherein v is 1 to 4.

Preferably, the amino acid units carrying R^{H1} are independently selected from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gin) unit, serine (Ser) unit, citrulline (Cit) unit and a phosphonomethylalanine (Pma) unit, more preferably from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, and a citrulline (Cit) unit, and still more preferably from a D-2,3-diaminopropionic acid unit, D-2,4-diaminobutanoic acid unit, D-ornithine unit, D-lysine unit, D-arginine unit, D-glutamic acid unit, D-aspartic acid unit, D-asparagine unit, D-glutamine unit, and a D-citrulline unit. Thus, for example, a preferred group —[C(O)—CH(R^{H1})—NH]ₘ₁-may consist of (i) two D-Asp units, (ii) two D-Orn units, (iii) two Asn units, or (iv) two D-Asp units and a third unit selected from D-Lys, D-Cit and D-Glu.

The variable d is an integer of 0 to 4, and e is an integer of 0 to 4. It is more preferred that one of d and e is 0, and the other one is 1 to 4, still more preferably the other one is 1. Thus, the most preferred combinations are d is 1 and e is 0, or d is 0 and e is 1.

The trivalent unit

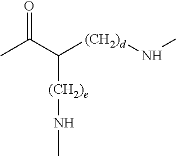

in the above formula is likewise an amino acid unit. Preferably, it is selected from a 2,3-diaminopropionic acid (Dap) unit, 2,4-diaminobutanoic acid (Dab) unit, ornithine (Orn) unit and a lysine (Lys) unit, more preferably a Dap unit. Still more preferred are a D-2,3-diaminopropionic acid (Dap) unit, D-2,4-diaminobutanoic acid (Dab) unit, D-ornithine (Orn) unit and a D-lysine (Lys) unit, most preferred is a D-Dap unit.

The unit —C(O)—CH(R^{H2})—NH—, in the above formula within the brackets [ . . . ]ₚ₁, likewise represents an amino acid unit, and R^{H2} is a side chain of the amino acid unit. The amino acid from which the amino acid unit is derived is preferably in D-configuration. R^{H2} is selected from —(CH₂)ᵥ—NH₂, —(CH₂)ᵥ—COOH, —(CH₂)ᵥ—NH—C(═NH)—NH₂, —(CH₂)ᵥ—C(═O)NH₂, —(CH₂)ᵥ—NH—C(═O)—NH₂, —(CH₂)ᵥ—OH and —(CH₂)ᵥ—P(═O)(OH)₂, wherein v is 1 to 4. Preferably, the amino acid unit carrying R^{H2} is selected from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, serine (Ser) unit, citrulline (Cit) unit and a phosphonomethylalanine (Pma) unit. Among them, a Dap unit is particularly preferred. More preferably the unit is selected from a D-2,3-diaminopropionic acid unit, D-2,4-diaminobutanoic acid unit, D-ornithine unit, D-lysine unit, D-arginine unit, D-glutamic acid unit, D-aspartic acid unit, D-asparagine unit, D-glutamine unit, D-serine unit, D-citrulline unit and a D-phosphonomethylalanine unit, a most preferred is a D-Dap unit.

The unit —C(O)—CH(R^{L2})—NH— within the brackets [ . . . ]_{q1} likewise represents an amino acid unit, and R^{L2} is H or a side chain of the amino acid unit. If R^{L2} is not H, the amino acid from which the amino acid unit is derived is preferably in D-configuration. R^{H2} is selected from H and CH₃, and is preferably H.

R^{S1} is selected from a group of formula (S-3) and a group of formula (S-4) as defined herein, and is more preferably a group (S-4):

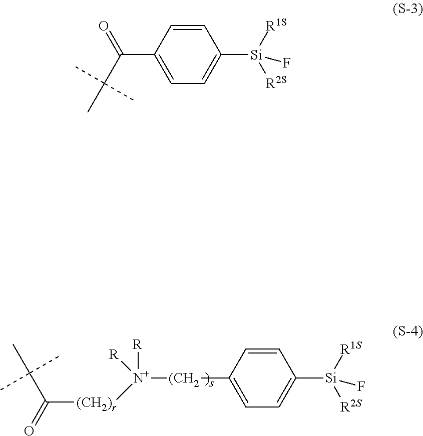

wherein
r is 1, 2 or 3, preferably 1, s is an integer of 1 to 6 and is preferably 1,
R is, independently, H or C1 to C6 alkyl, preferably H or C1 to C2 alkyl, and is more preferably methyl, and
R^{1S} and R^{2S} are independently from each other a linear or branched C3 to C10 alkyl group, preferably R^{1S} and R^{2S} are selected from isopropyl and tert-butyl, and more preferably R^{1S} and R^{2S} are tert-butyl; and wherein the dashed line marks a bond which attaches the group to the remainder of the compound.

As noted above, among the groups (S-3), a group (S-3a) is strongly preferred, and among the groups (S-4), the group (S-4a) is strongly preferred.

A preferred type of compound of formula (Ia) or a salt thereof is one of the formula (Ia.1) or a salt thereof:

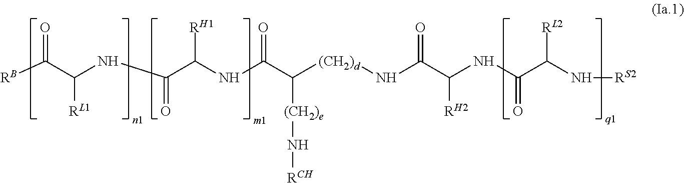

wherein R^{B}, R^{L1}, n1, R^{H1}, m1, d, e, R^{CH}, R^{H2}, R^{L2} and q1 are as defined herein above, including their preferred embodiments, and R^{S2} is a SiFA group of the formula (S-3), more preferably (S-3a) as defined herein above, i.e.:

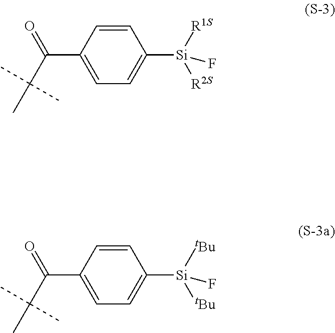

wherein R^{1S} and R^{2S} are as defined herein above, including their preferred embodiments.

Among the compounds of formula (Ia.1) or their salts, further preference is given to those of formula (Ia.2) or their salts:

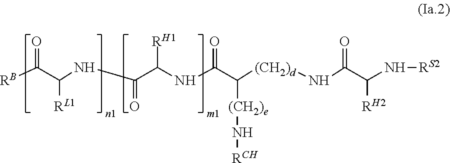

wherein R^{B}, R^{L1}, n1, R^{H1}, m1, d, e, R^{CH}, R^{H2} and R^{S2} are as defined herein above, including their preferred embodiments.

A still more preferred type of the compound of formula (Ia) or a salt thereof is one of the formula (Ia.3) or a salt thereof:

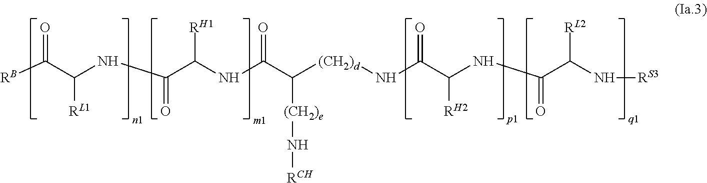

wherein R^{B}, R^{L1}, n1, R^{H1}, m1, d, e, R^{CH}, R^{H2}, p1, R^{L2} and q1 are as defined herein above, including their preferred embodiments, and R^{S3} is a SiFA group of the formula (S-4), more preferably (S-4a) as defined herein above, i.e.:

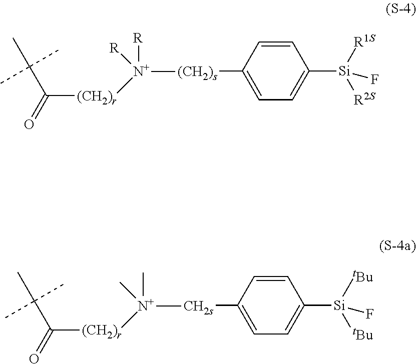

wherein r, s, R, R^{1S} and R^{2S} are as defined herein, including their preferred embodiments.

Among the compounds of formula (Ia.3) or their salts, further preference is given to those of formula (Ia.4) and (Ia.5) or their salts:

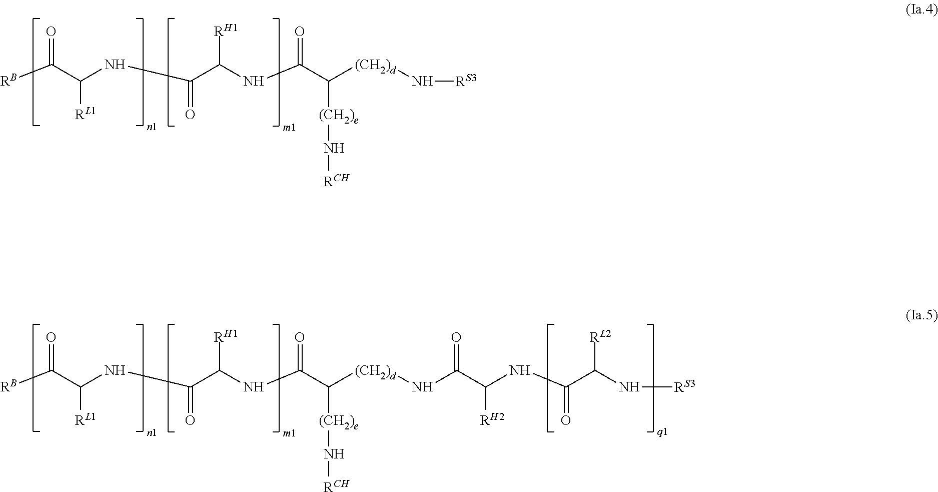

wherein Re, R^{L1}, n1, R^{H1}, m1, d, e, R^{C}H, R^{H2}, R^{L2}, q1 and R^{S3} are as defined herein above, including their preferred embodiments.

As noted above, the compounds in accordance with the invention encompass the compounds of formula (I) and their salts. Salts are preferably pharmaceutically acceptable salts, i.e. formed with pharmaceutically acceptable anions or cations. Salts may be formed, e.g., by protonation of an atom carrying an electron lone pair which is susceptible to protonation, such as a nitrogen atom, with an inorganic or organic acid, or by separating a proton from an acidic group, such as a carboxylic acid group, e.g. by neutralization with a base. Other charged groups which may be present in the compounds in accordance with the invention and which may provide the compounds in the form of a salt include groups which are continuously charged, such as a quaternary ammonium group comprising an ammonium cation wherein the nitrogen is substituted by four organyl groups, or charged chelate complexes.

As exemplary anions which may be present as counterions in salt forms of the compounds of the invention if the salt form comprises a positively charged form of the compound of formula (I), mention may be made, for example, of an anion selected from chloride, bromide, iodide, sulfate, nitrate, phosphate (such as, e.g., phosphate, hydrogenphosphate, or dihydrogenphosphate salts), carbonate, hydrogencarbonate or perchlorate; acetate, trifluoroacetate, propionate, butyrate, pentanoate, hexanoate, heptanoate, octanoate, cyclopentanepropionate, undecanoate, lactate, maleate, oxalate, fumarate, tartrate, malate, citrate, nicotinate, benzoate, salicylate or ascorbate; sulfonates such as methanesulfonate, ethanesulfonate, 2-hydroxyethanesulfonate, benzenesulfonate, p-toluenesulfonate (tosylate), 2-naphthalenesulfonate, 3-phenylsulfonate, or camphorsulfonate. Since trifluoroacetic acid is frequently used during the synthesis of peptides, trifluoroacetate salts are typical salts which are provided if a compound comprising a peptide structure is formed. Such trifluoroacetate salts may be converted e.g. to acetate salts during their workup.

As exemplary cations which may be present as counterions in salt forms of the compounds of the invention if the salt form comprises a negatively charged form of the compound of formula (I), mention may be made, for example, of a cation selected from alkali metal cations, such as lithium, sodium or potassium, alkaline earth metal cations, such as calcium or magnesium; and ammonium (including ammonium ions substituted by organic groups).

The compound in accordance with the invention is preferably capable of binding to an SST receptor, preferably to SST₂, with an affinity reflected by an IC₅₀ value of 50 nM or less, more preferably 10 nM or less, still more preferably 5 nM or less.

The compound in accordance with the invention preferably exhibits an octanol-water distribution coefficient (also referred to as log D_{7.4} or log P value), of −1.0 or less, more preferably −2.0 or less. It is generally not below −4.0.

This distribution coefficient may be determined by measuring the equilibrium distribution, e.g. at room temperature (20° C.) of a compound in accordance with the invention in a two-phase system containing equal amounts, such as 1.00 m1 each, of n-octanol and PBS (pH=7.4), and calculating the log D₇₄ value as log₁₀(concentration in octanol/concentration in PBS). Instead of the (absolute) concentration of the compound in accordance with the invention in the octanol and the PBS, a parameter which is proportional to the concentration of the compound in each phase may also be used for the calculation, such as the activity of radiation if the compound comprises a radioactive moiety, e.g. a radioactive chelate.

The compounds of the invention can provide advantageous binding characteristics to human serum albumin (HSA). Moderate to low HSA binding values, expressed as the apparent molecular weight in kDa and determined via radio inversed affinity chromatography (RIAC) as described in the examples section below can be achieved. Preferably, the HSA binding value is less than 22 kDa, more preferably below 10 kDa.

As exemplary compounds in accordance with the invention, the following are further mentioned.

Compound 23 having the formula shown in the Examples section below, a compound wherein the DOTA chelating group shown in the formula forms a chelate with a chelated radioactive or non-radioactive cation, or any salt thereof.

Compound 19 having the formula shown in the Examples section below, a compound wherein the DOTA chelating group shown in the formula forms a chelate with a chelated radioactive or non-radioactive cation, or any salt thereof.

Compound 39 having the formula shown in the Examples section below, a compound wherein the DOTA chelating group shown in the formula forms a chelate with a chelated radioactive or non-radioactive cation, or any salt thereof.

Compound 40 having the formula shown in the Examples section below, a compound wherein the DOTA chelating group shown in the formula forms a chelate with a chelated radioactive or non-radioactive cation, or any salt thereof.

Compound 20 having the formula shown in the Examples section below, a compound wherein the DOTAGA chelating group shown in the formula forms a chelate with a chelated radioactive or non-radioactive cation, or any salt thereof.

Compound 41 having the formula shown in the Examples section below, a compound wherein the DOTAGA chelating group shown in the formula forms a chelate with a chelated radioactive or non-radioactive cation, or any salt thereof.

Compound 42 having the formula shown in the Examples section below, a compound wherein the DOTA chelating group shown in the formula forms a chelate with a chelated radioactive or non-radioactive cation, or any salt thereof.

Compound 42 having the formula shown in the Examples section below, a compound wherein the DOTA chelating group shown in the formula forms a chelate with a chelated radioactive or non-radioactive cation, or any salt thereof.

Compound 36 having the formula shown in the Examples section below, a compound wherein the DO3AM chelating group shown in the formula forms a chelate with a chelated radioactive or non-radioactive cation, or any salt thereof.

Compound 37 having the formula shown in the Examples section below, a compound wherein the DOTAGA chelating group shown in the formula forms a chelate with a chelated radioactive or non-radioactive cation, or any salt thereof.

Compound 24 having the formula shown in the Examples section below, a compound wherein the DOTA chelating group shown in the formula forms a chelate with a chelated radioactive or non-radioactive cation, or any salt thereof.

Compound 38 having the formula shown in the Examples section below, a compound wherein the DO3AM chelating group shown in the formula forms a chelate with a chelated radioactive or non-radioactive cation, or any salt thereof.

Compound 48 having the formula shown in the Examples section below, a compound wherein the DOTA chelating group shown in the formula forms a chelate with a chelated radioactive or non-radioactive cation, or any salt thereof.

Compound 49 having the formula shown in the Examples section below, a compound wherein the DOTA chelating group shown in the formula forms a chelate with a chelated radioactive or non-radioactive cation, or any salt thereof.

Compound 50 having the formula shown in the Examples section below, a compound wherein the DOTA chelating group shown in the formula forms a chelate with a chelated radioactive or non-radioactive cation, or any salt thereof.

Compound 54 having the formula shown in the Examples section below, a compound wherein the DOTAM (or DO3AM) chelating group shown in the formula forms a chelate with a chelated radioactive or non-radioactive cation, or any salt thereof.

Compound 55 having the formula shown in the Examples section below, a compound wherein the DOTA chelating group shown in the formula forms a chelate with a chelated radioactive or non-radioactive cation, or any salt thereof.

Compound 56 having the formula shown in the Examples section below, a compound wherein the DOTA chelating group shown in the formula forms a chelate with a chelated radioactive or non-radioactive cation, or any salt thereof.

Compound 57 having the formula shown in the Examples section below, a compound wherein the DOTA chelating group shown in the formula forms a chelate with a chelated radioactive or non-radioactive cation, or any salt thereof.

Compound 58 having the formula shown in the Examples section below, a compound wherein the DOTA chelating group shown in the formula forms a chelate with a chelated radioactive or non-radioactive cation, or any salt thereof.

Compound 59 having the formula shown in the Examples section below, a compound wherein the DOTA chelating group shown in the formula forms a chelate with a chelated radioactive or non-radioactive cation, or any salt thereof.

Compound 61 having the formula shown in the Examples section below, a compound wherein the DOTAM (or DO3AM) chelating group shown in the formula forms a chelate with a chelated radioactive or non-radioactive cation, or any salt thereof.

Compound 44 having the formula shown in the Examples section below, a compound wherein the DOTA chelating group shown in the formula forms a chelate with a chelated radioactive or non-radioactive cation, or any salt thereof.

Compound 45 having the formula shown in the Examples section below, a compound wherein the DOTA chelating group shown in the formula forms a chelate with a chelated radioactive or non-radioactive cation, or any salt thereof.

Compound 46 having the formula shown in the Examples section below, a compound wherein the DOTA chelating group shown in the formula forms a chelate with a chelated radioactive or non-radioactive cation, or any salt thereof.

Compound 47 having the formula shown in the Examples section below, a compound wherein the DOTA chelating group shown in the formula forms a chelate with a chelated radioactive or non-radioactive cation, or any salt thereof.

Compound 51 having the formula shown in the Examples section below, a compound wherein the DOTA chelating group shown in the formula forms a chelate with a chelated radioactive or non-radioactive cation, or any salt thereof.

Compound 60 having the formula shown in the Examples section below, a compound wherein the DOTA chelating group shown in the formula forms a chelate with a chelated radioactive or non-radioactive cation, or any salt thereof.

Compound 52 having the formula shown in the Examples section below, a compound wherein the DOTA chelating group shown in the formula forms a chelate with a chelated radioactive or non-radioactive cation, or any salt thereof.

Compound 53 having the formula shown in the Examples section below, a compound wherein the DOTA chelating group shown in the formula forms a chelate with a chelated radioactive or non-radioactive cation, or any salt thereof.

To the extent that these exemplary compounds or salts comprise a chelated radioactive or non-radioactive cation, the cation is preferably a cation of Ga, Lu, or Pb.

In a further aspect, the present invention provides a pharmaceutical composition (also referred to as a therapeutic composition) comprising or consisting of one or more types, preferably one type, of the ligand compound in accordance with the invention, i.e. a compound of formula (I) including any preferred embodiments thereof as discussed herein or a salt thereof. In a related aspect, the ligand compound in accordance with the invention is provided for use in therapy or for use as a medicament. Thus, the ligand compound of the invention can be used in a therapeutic method, which method may comprise administering the ligand compound to a subject. The subject may be a human or an animal and is preferably a human.

The therapy or therapeutic method referred to above aims at the treatment or prevention of a disease or disorder of the human or animal body, generally a disease or disorder that is associated with increased or aberrant expression of a somatostatin receptor, preferably a disease or disorder associated with increased or aberrant expression of SST₂. The disease or disorder to be treated or prevented can be cancer, preferably a neuroendocrine tumor.

For example, a compound in accordance with the invention comprising a chelated radioactive cation, such as a ¹⁷⁷Lu cation or a ⁶⁸Ga cation, can be advantageously used in radiotherapy, such as the radiotherapy of a disease or disorder as discussed above.

In another aspect, the present invention provides a diagnostic composition comprising or consisting of one or more types, preferably one type, of the ligand compound in accordance with the invention, i.e. a compound of formula (I) including any preferred embodiments thereof as discussed herein or a salt thereof. In a related aspect, the ligand compound in accordance with the invention is provided for use in a method of diagnosis in vivo of a disease or disorder.

Thus, the ligand compound in accordance with the invention can be used in a method of diagnosis, which method may comprise administering the ligand compound to a subject and detecting the ligand compound in the subject, or monitoring the distribution of the ligand compound in the subject thereby detecting or monitoring the disease to be diagnosed. For example, nuclear imaging by means of Positron Emission Tomography (PET) or Single Photon Emission Computed Tomography (SPECT), respectively, can be used for detecting or monitoring a ligand compound in accordance with the invention. The subject may be a human or an animal and is preferably human. Alternatively, a method of diagnosis may also comprise adding the ligand compound to a sample, e.g. a physiological sample obtained from a subject in vitro or ex vivo, and detecting the ligand compound in the sample.

The method of diagnosis referred to above aims at the identification of a disease or disorder of the human or animal body, generally a disease or disorder that is associated with increased or aberrant expression of a somatostatin receptor, preferably a disease or disorder associated with increased or aberrant expression of SST₂. Thus, in terms of a diagnostic application, the compounds of the invention are preferably provided for use in a method of diagnosis in vivo of cancer, more preferably a neuroendocrine tumor.

For example, a compound of the invention wherein the SiFA group comprises a ⁸F fluoride, or a compound of the invention wherein the chelating groups comprises a chelated radioactive cation, e.g. a ⁶⁸Ga cation, can be advantageously used for nuclear diagnostic imaging, such as diagnosis via positron emission tomography (PET) or via Single Photon Emission Computed Tomography (SPECT).

It will be understood that suitability for a therapeutic and a diagnostic application is not mutually exclusive. i.e. a compound in accordance with the invention may be suitable for both applications. For example, a compound comprising a chelated ¹⁷⁷Lu cation can be used both for therapeutic and diagnostic imaging applications. Moreover, due to the presence of a chelating group and a SiFA group, the compounds of the invention are suitable as radiohybrid (rh) ligands. Such a rh ligand can be alternatively labeled with [⁹⁸F] fluoride (e.g. for PET) or a radiometal (such as a ⁶⁸Ga cation for PET, or a ¹⁷⁷Lu cation for radiotherapy). When a rh ligand is labeled with [¹⁸F]fluoride, a cold (non-radioactive) metal cation can, but not necessarily must be complexed elsewhere in the molecule, and when it is labeled with a corresponding radioactive metal cation, cold [¹⁸F] fluorine can be included. Therefore, the ¹⁸F-labeled peptide and the corresponding radiometal-labeled analog can possess the same chemical structure and thus identical in vitro and in vivo properties, thereby allowing the generation of structurally identical theranostic tracers with exactly the same in vivo properties of the diagnostic and therapeutic tracers (e.g. ¹⁸F/¹⁷⁷Lu analogs) [24].

Thus, in line with this approach the ligand compounds of the invention include compounds wherein the silicon-fluoride acceptor group is labeled with ¹⁸F and the chelating group contains a chelated non-radioactive cation (such as ^{nat}Lu or ^{nat}Ga), and compounds wherein the chelating group contains a chelated radioactive cation (such as ¹⁷⁷Lu or ⁶⁸Ga) and the silicon-fluoride acceptor group is not labeled with ¹⁸F (thus carrying a ¹⁹F). Likewise, the invention provides the compounds of the invention for use in a hybrid method of diagnosis in vivo and therapy of a disease or disorder associated with increased or aberrant expression of a somatostatin receptor as discussed above, wherein the method involves first the administration of a compound of the invention wherein the silicon-fluoride acceptor group is labeled with ¹⁸F and the chelating group contains a chelated non-radioactive cation (such as ^{nat}Lu or ^{nat}Ga), and subsequently of a compound wherein the chelating group contains a chelated radioactive cation and the silicon-fluoride acceptor group is not labeled with ^{1I}F.

Thus, in another aspect, the present invention provides a dedicated composition comprising or consisting of one or more types, preferably one type, of the ligand compound in accordance with the invention, i.e. a compound of formula (I) including any preferred embodiments thereof as discussed herein, or a salt thereof. In a related aspect, the ligand compound in accordance with the invention is provided for use in a method of in vivo imaging of a disease or disorder.

Thus, the ligand compound in accordance with the invention can be used in an imaging method, which method may comprise administering the ligand compound to a subject and detecting the ligand compound in the subject and monitoring the distribution of the ligand compound in vivo at different time points after injection with the aim to calculate the dosimetry prior or during a therapeutic treatment. The subject may be a human or an animal and is preferably human.

The imaging method referred to above aims at the calculation of the dosimetry prior or during a therapeutic treatment of a disease or disorder of the human or animal body, generally a disease or disorder that is associated with increased or aberrant expression of a somatostatin receptor, preferably a disease or disorder associated with increased or aberrant expression of SST₂. Thus, in terms of such application, the compounds of the invention are preferably provided for use in an in vivo imaging method for cancer, more preferably a neuroendocrine tumor.

For example, a compound of the invention wherein the SiFA group comprises a ¹⁸F fluoride and non-radioactive ^{nat}Lu, or a compound of the invention wherein the chelating group comprises a chelated radioactive cation, e.g. a ¹⁷Lu cation, whereas the SiFA is non-radioactive, can be advantageously used for nuclear imaging by means of Positron Emission Tomography (PET) or Single Photon Emission Computed Tomography (SPECT), respectively, to monitor the distribution of the applied compound and thereafter calculate the individual dosimetry by means of the quantitative distribution kinetics.

The pharmaceutical or diagnostic composition may further comprise one or more pharmaceutically acceptable carriers, excipients and/or diluents. Examples of suitable pharmaceutical carriers, excipients and/or diluents are well known in the art and include phosphate buffered saline solutions, amino acid buffered solutions (with or without saline), water for injection, emulsions, such as oil/water emulsions, various types of wetting agents, sterile solutions etc. Compositions comprising such carriers can be formulated by well-known conventional methods. These compositions can be administered to the subject at a suitable dose. Administration of the suitable compositions may be accomplished in different ways, e.g., by intravenous, intraperitoneal, subcutaneous, intramuscular, topical, intradermal, intranasal or intrabronchial administration. It is particularly preferred that said administration is carried out by intravenous injection and/or delivery. The compositions may be administered directly to the target site. The dosage regimen will be determined by the attending physician and clinical factors. As is well known in the medical arts, dosages for any one patient depends upon many factors, including the patient's size, body surface area, age, the particular compound to be administered, dosimetry, sex, time and route of administration, general health, and other drugs being administered concurrently. The compounds may be administered e.g. in amounts between 0.1 ng and 10 μg/kg body weight. For example, in diagnostic applications, a typical dosage amount of the compounds of the invention or their salts is <100 μg/patient, e.g. in the range of 0.1 to 30 μg/patient, however, if appropriate, higher or lower dosages can be envisaged. A typical dosage amount of the compounds of the invention or their salts in a radiotherapeutic application is in the range of 50 to 200 μg/patient, preferably 75 to 150 μg/patient, however, if appropriate, higher or lower dosages can be envisaged.

The following items summarize aspects of the invention. It will be understood that these items are closely related to the above parts of the description, and that the information provided in these items may supplement the above parts of the description and vice versa.
1. A compound of formula (I) or a salt thereof:

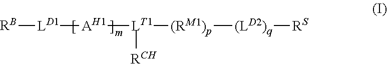

wherein:
R^{B} is a binding motif which is able to bind to a somatostatin receptor;
L^{D1} is a divalent linking group;
m is an integer of 2 to 6, preferably 2 to 5, more preferably 2 or 3;
A^{A1} is, independently for each occurrence, an amino acid unit derived from a hydrophilic amino acid which comprises, in addition to its —NH₂ and its —COOH functional group, a further hydrophilic functional group,
and wherein one of the m units A^{A1} may further carry a hydrophilic unit other than an amino acid bound to its hydrophilic functional group;
L^{T1} is a trivalent linking group;
R^{CH} is a chelating group which optionally contains a chelated radioactive or non-radioactive cation;
R^{M1} is a hydrophilic modifying group, and p is 0 or 1;
L^{D2} is a divalent linking group, and q is 0 or 1; and
R^{S} is a silicon-fluoride acceptor (SiFA) group of one of the following formulae, which comprises a silicon atom and a fluorine atom, and which can be labeled with ¹⁸F by isotopic exchange of ¹⁹F by ¹⁸F or which is labeled with ¹⁸F:
if p is 1, R^{S} is a group of formula (S-3) or a group of formula (S-4):

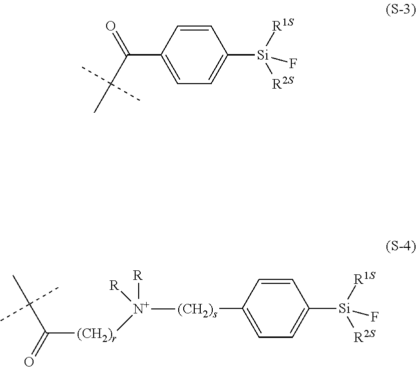

wherein
r is 1, 2 or 3, preferably 1, s in —(CH₂)ₛ— is an integer of 1 to 6 and is preferably 1, the groups R are, independently, H or C1 to C6 alkyl, preferably H or C1 to C2 alkyl, and are more preferably both methyl, and
R^{1S} and R^{2S} are independently from each other a linear or branched C3 to C10 alkyl group, preferably R^{1S} and R^{2S} are selected from isopropyl and tert-butyl, and more preferably R^{1S} and R^{2S} are tert-butyl; and wherein the dashed line marks a bond which attaches the group to the remainder of the compound; and
if p is 0, R^{S} is a group of formula (S-4) as defined above.
2. The compound or salt of item 1, wherein the binding motif is able to bind to the somatostatin receptor 2.
3. The compound or salt of item 1 or 2, wherein the binding motif R^{B} is a group which can be derived from a receptor agonist or receptor antagonist selected from Tyr³,Thr⁸-Octreotide (TATE), Tyr³-Octreotide (TOC), Thr⁸-Octreotide (ATE); 1-Nal³-Octreotide (NOC), 1-Nal³,Thr⁸-octreotide (NOCATE), BzThi³-octreotide (BOC), BzThi³,Thr⁸-octreotide (BOCATE), JR11, BASS, and KE121.
4. The compound or salt of item 1 or 2, wherein the binding motif R^{B} is a group of the formula (B-1) or (B-2):

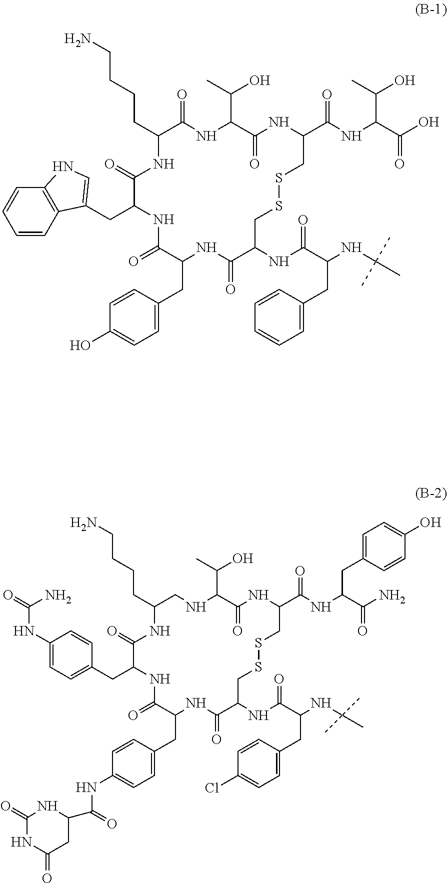

wherein the dashed line marks a bond which attaches the group to the remainder of the compound.
5. The compound or salt of any of items 1 to 4, wherein the chelating group R^{CH} comprises at least one of
(i) a macrocyclic ring structure with 8 to 20 ring atoms of which 2 or more, preferably 3 or more, are selected from oxygen atoms and nitrogen atoms; and
(ii) an acyclic, open chain chelating structure with 8 to 20 main chain atoms of which 2 or more, preferably 3 or more are heteroatoms selected from oxygen atoms and nitrogen atoms.
6. The compound or salt in accordance with any of items 1 to 5, wherein the chelating group R^{CH} is a group which can be derived from a chelating agent selected from diethylenetriaminepentamethylenephosphonic acid (EDTMP) and its derivatives, diethylenetriaminepentaacetic acid (DTPA) and its derivatives, bis(carboxymethyl)-1,4,8,11-tetraaza-bicyclo[6.6.2] hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino]¬pentyl]-N-[5-[[4-[5-aminopentyl-(hydroxy)amino]-4-oxobutanoyl]-amino]pentyl]-N-hydroxybutandiamide (DFO) and derivatives thereof, 1,4,7,10-tetraazacyclododecane-1,7-diacetic acid (DO2A), 1,4,7,10-tetraazacyclododecan-N,N′,N″,N′″-tetraacetic acid (DOTA), 2-[1,4,7,10-tetraazacyclododecane-4,7,10-triacetic acid]-pentanedioic acid (DOTAGA or DOTA-GA), 1,4,7,10-tetrakis(carbamoylmethyl)-1,4,7,10-tetraazacyclododecane (DOTAM), N,N′-dipyridoxylethylendiamine-N,N′-diacetate-5,5′-bis(phosphat) (DPDP), diethylenetriaminepentaacetic acid (DTPA), ethylenediamine-N,N′-tetraacetic acid (EDTA), ethyleneglykol-O,O-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclodecan-4,7,10-triacetate (HP-DOA3), 6-hydrazinyl-N-methylpyridine-3-carboxamide (HYNIC), 1,4,7-triazacyclononan-1-succinic acid-4,7-diacetic acid (NODASA), 1-(1-carboxy-3-carboxypropyl)-4,7-(carboxy)-1,4,7-triazacyclononane (NODAGA), 1,4,7-triazacyclononanetriacetic acid (NOTA), 4,11-bis(carboxymethyl)-1,4,8,11-tetraazabicyclo[6.6.2]hexadecane (TE2A), 1,4,8,11-tetraazacyclododecane-1,4,8,11-tetraacetic acid (TETA), terpyridine-bis(methyleneamine) tetraacetic acid (TMT), 1,4,7,10-tetraazacyclotridecan-N,N′,N″,N′″-tetraacetic acid (TRITA), and triethylenetetraaminehexaacetic acid (TTHA), N,N′-bis[(6-carboxy-2-pyridil)methyl]-4,13-diaza-18-crown-6 (H₂macropa), 4-amino-4-{2-[(3-hydroxy-1,6-dimethyl-4-oxo-1,4-dihydro-pyridin-2-ylmethyl)-carbamoyl]-ethyl} heptanedioic acid bis-[(3-hydroxy-1,6-dimethyl-4-oxo-1,4-dihydro-pyridin-2-ylmethyl)-amide] (THP), 1,4,7-triazacyclononane-1,4,7-tris[methylene(2-carboxyethyl)phosphinic acid (TRAP), 2-(4,7,10-tris(2-amino-2-oxoethyl)-1,4,7,10-tetraazacyclododecan-1-yl)acetic acid (DO3AM), and 1,4,7,10-tetraazacyclododecane-1,4,7,10-tetrakis[methylene(2-carboxyethylphosphinic acid)](DOTPI), S-2-(4-isothiocyanatobenzyl)-1,4,7,10-tetraazacyclododecane tetraacetic acid, hydrazinonicotinic acid (HYNIC), 6-amino-6-methylperhydro-1,4-diazepine-N,N,N′,N′-tetraacetic acid (AAZTA) and derivatives thereof, such as (6-pentanoic acid)-6-(amino)methyl-1,4-diazepine triacetate (DATA), pentadeca-1,4,7,10,13-penta-aminopentaacetic acid (PEPA), hexadeca-1,4,7,10,13,16-hexaamine-hexaacetic acid (HEHR), 4-{[bis(phosphonomethyl)) carbamoyl] methyl}-7,10-bis(carboxymethyl)-1,4,7,10-tetraazacyclododec-1-yl) acetic acid (BPAMD), N-(4-{[bis (phosphonomethyl)) carbamoyl] methyl}-7,10-bis (carboxymethyl)-nona-1,4,7-triamine triacetic acid (BPAM), 1,2-[{6-(carboxylate) pyridin-2-yl} methylamine] ethane (DEDPA, H₂DEDPA), deferoxamine (DFO) and its derivatives, deferiprone, (4-acetylamino-4-yl) {2-[(3-hydroxy-1,6-dimethyl-4-oxo-1,4-dihydro-pyridin-2-ylmethyl)-carbamoyl]-ethyl}-heptanedioic acid bis-[(3-hydroxy-1,6-dimethyl-4-oxo-1,4-dihydro-pyridin-2-ylmethyl)-amide](CP256) and its derivatives such as YM103; tetraazycyclodecane-phosphinic acid (TEAP), 6-amino-6-methylperhydro-1,4-diazepine-N,N,N′,N′-tetraacetic acid (AAZTA); 1-N-(4-aminobenzyl)-3,6,10,13,16,19-hexaazabicyclo [6.6.6]-eicosane-1,8-diamine (SarAr), 6,6′-[{9-hydroxy-1,5-bis-(methoxycarbonyl)-2,4-di(pyridin-2-yl)-3,7-diazabicyclo[3.3.1]nonane-3,7-diyl}bis(methylene)]dipicolinic acid (H₂bispa²), 1,2-[{6-(carboxylato)pyridin-2-yl}methylamino]-ethane (H₂dedpa), N,N′-bis(6-carboxy-2-pyridylmethyl)-ethylenediamine-N,N′-diacetic acid (H₄octapa), N,N′-bis(2-hydroxy-5-sulfonylbenzyl)-N,N′-bis-(2-methylpyridyl)ethylenediamine (H₆Sbbpen) and derivatives thereof, triethylenetetramine-N,N,N′,N″,N′″,N′″-hexaacetic (TTHA), 2-aminomethylpiperidine triacetic acid (2-AMPTA) and derivatives thereof, such as 2-(N-(2-Hydroxybenzyl)aminomethyl)piperidine (2-AMPTA-HB) further functionalized derivative of 2-AMPTA with additional functional groups suitable for conjugation to peptidic structures, 4-nitro-2-hydroxybenzyl-2-{[(6)-trans-2-[benzyl(carboxymethyl)amino]cyclohexyl](carboxymethyl)amino}acetic acid (RESCA) and derivatives thereof, and 6-carboxy-1,4,8,11-tetraazaundecane (N₄) and derivatives thereof, and wherein the chelating group optionally comprises a chelated radioactive or non-radioactive cation.
7. The compound or salt of item 6, wherein the chelating group R^{CH} is a group which can be derived from a chelating agent selected from CBTE2a, DOTA, DOTAGA, DOTAM, NODASA, NODAGA, NOTA, TE2A, DO3AM, SarAr, 2-AMPTA, 2-AMPTA-HB and RESCA, and wherein the chelating group optionally comprises a chelated radioactive or non-radioactive cation.
8. The compound or salt of item 7, wherein the chelating group R^{CH} is a group which can be derived from a chelating agent selected from DOTA, DOTAGA, DOTAM, DO3AM, NOTA and NODAGA, and wherein the chelating group optionally comprises a chelated radioactive or non-radioactive cation.
9. The compound or salt of any of items 1 to 8, wherein R^{CH} is a group of the formula (CH-1), (CH-2) or (CH-3) which optionally contains a chelated radioactive or non-radioactive cation:

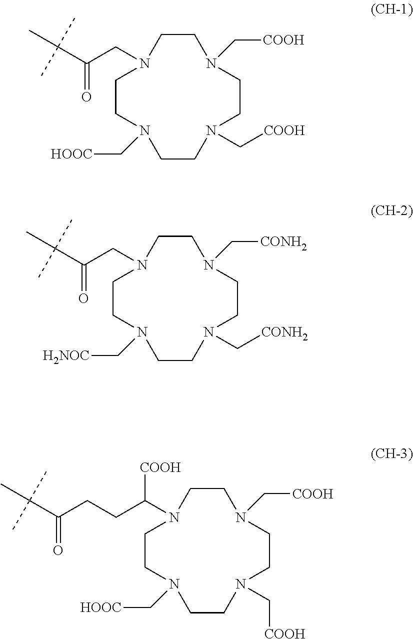

wherein the dashed line marks a bond which attaches the group to the remainder of the compound.
10. The compound or salt of any of items 1 to 9, wherein the chelated cation, if present, comprises or consists of a cation selected from cations of ⁴³Sc, ⁴⁴Sc, ⁴⁷Sc, ⁵¹Cr, ^{52m}Mn, ⁵⁵Co, ⁵⁷Co, ⁵⁸Co, ⁵²Fe, ⁵⁶Ni, ⁵⁷Ni, ⁶²Cu, ⁶⁴Cu, ⁶⁷Cu, ⁶⁶Ga, ⁶⁸Ga, ⁶⁷Ga, ⁸⁹Zr, ⁹⁰Y, ⁸⁶Y, ^{94m}Tc, ^{99m}Tc, ⁹⁷Ru, ¹⁰⁵Rh, ¹⁰⁹Pd, ¹¹¹Ag, ^{110m}In, ¹¹¹In, ^{113m}In, ^{114m}In, ^{117m}Sn, ¹²¹Sn, ¹²⁷Te, ¹⁴²Pr, ¹⁴³Pr, ¹⁴⁷Nd, ¹⁴⁹Gd, ¹⁴⁹Pm, ¹⁵¹Pm, ¹⁴⁹Tb, ¹⁵²Tb, ¹⁵⁵Tb, ¹⁵³Sm, ¹⁵⁶Eu, ¹⁵⁷Gd, ¹⁵⁵Tb, ¹⁶¹Tb, ¹⁶⁴Tb, ¹⁶¹Ho, ¹⁶⁶Ho, ¹⁵⁷Dy, ¹⁶⁵Dy ¹⁶⁶Dy, ¹⁶⁰Er, ¹⁶⁵Er, ¹⁶⁹Er, ¹⁷¹Er, ¹⁶⁶Yb, ¹⁶⁹Yb, ¹⁷⁵Yb, ¹⁶⁷Tm, ¹⁷²Tm, ¹⁷⁷Lu, ¹⁸⁶Re, ^{186g}Re, ¹⁸⁸Re, ¹⁸⁸W, ¹⁹¹Pt, ^{195m}Pt, ¹⁹⁴Ir, ¹⁹⁷Hg, ¹⁹⁸Au, ¹⁹⁹Au, ²¹²Pb, ²⁰³Pb, ²¹¹At, ²¹²Bi, ²¹³Bi, ²²³Ra, ²²⁴Ra, ²²⁵Ac, ²²⁶Th and ²²⁷Th, and from nonradioactive isotopes of any of these metals, or is a cationic molecule comprising ¹⁸F or ¹⁸F, such as ¹⁸F—[AlF]²⁺.
11. The compound or salt of any of items 1 to 10, wherein the divalent linking group L^{D1} comprises a divalent oligo- or polyethylene glycol group; preferably a divalent oligo- or polyethylene glycol group having 10 or less ethylene glycol units, and more preferably a divalent oligo- or polyethylene glycol group having 2 to 5 ethylene glycol units.
12. The compound or salt of item 11, wherein L^{D1} is a divalent group of formula (L-1a):

—C(O)—(CH₂)ₐ—(O—CH₂—CH₂)_{b}—NH— (L-1a)
wherein
a is 1 or 2, preferably 1;
b is an integer of 2 to 5, preferably 2 or 3.
13. The compound or salt of any of items 1 to 10, wherein the divalent linking group L^{D1} comprises a divalent amino acid unit or a divalent chain of amino acid units, more preferably a divalent chain of 2 to 5 amino acid units, still more preferably a divalent chain of 2 or 3 amino acid units
14. The compound or salt of item 13, wherein L^{D1} is represented by formula (L-1b):

-[A^{L1}]ₙ- (L-1 b)
wherein
A^{L1} is, independently for each occurrence if n is more than 1, an amino acid unit; and
n is an integer of 1 to 5, preferably 2 to 5, more preferably 2 or 3.
15. The compound or salt of item 13 or 14, wherein the amino acid units in L^{D1} do not contain any free amino groups, free acid groups, or salts thereof.
16. The compound or salt of any of items 13 to 15, wherein each amino acid unit in L^{D1} is selected, independently for each occurrence if more than one amino acid unit is present in L^{D1}, from a glycine (Gly) unit, alanine (Ala) unit, ß-alanine unit, asparagine (Asn) unit, glutamine (Gln) unit, and citrulline (Cit) unit.
17. The compound or salt of any of items 1 to 16, wherein the further hydrophilic functional group of the amino acid units A^{A1} are selected, independently for each occurrence, from —NH₂, —COOH, —NH—C(═NH)—NH₂, —C(═O)NH₂, —NH—C(═O)—NH₂, —OH and —P(═O)(OH)₂.
18. The compound or salt of any of items 1 to 17, wherein each of the m amino acid units A^{H1} comprises, independently for each occurrence, a side chain having a terminal hydrophilic functional group, which side chain is selected from —(CH₂)ᵥ—NH₂, —(CH₂)ᵥ—COOH, —(CH₂)ᵥ—NH—C(═NH)—NH₂, —(CH₂)ᵥ—C(═O)NH₂, —(CH₂)ᵥ—NH—C(═O)—NH₂, —(CH₂)ᵥ—OH and —(CH₂)ᵥ—P(═O)(OH)₂ wherein v is 1 to 4, and wherein the terminal functional group of the above side chain in one of the m units A^{A1} may form a bond with an additional hydrophilic unit other than an amino acid unit.
19. The compound or salt of item 18, wherein each of the m amino acid units A^{A1} comprises, independently for each occurrence, a side chain having a terminal hydrophilic functional group, which side chain is selected from —(CH₂)ᵥ—NH₂, —(CH₂)ᵥ—COOH, —(CH₂)ᵥ—NH—C(═NH)—NH₂, —(CH₂)—C(═O)NH₂, and —(CH₂)ᵥ—NH—C(═O)—NH₂, wherein v is 1 to 4, and wherein the terminal functional group of the above side chain in one of the m units A^{A1} may form a bond with an additional hydrophilic unit other than an amino acid unit.
20. The compound or salt of any of items 1 to 18, wherein the amino acid units A^{M1} are selected, independently for each occurrence, from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, serine (Ser) unit, citrulline (Cit) unit and a phosphonomethylalanine (Pma) unit.
21. The compound or salt of item 20, wherein the amino acid units A^{A1} are selected, independently for each occurrence, from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, and a citrulline (Cit) unit.
22. The compound or salt of any of items 1 to 21, wherein the hydrophilic unit other than the amino acid unit which is optionally bound to A^{H1} is a carbohydrate group or a trimesic acid group.
23. The compound or salt of any of items 1 to 22, wherein L^{T1} is a trivalent amino acid unit selected from
(i) a 2,3-diaminopropionic acid (Dap) unit, 2,4-diaminobutanoic acid (Dab) unit, ornithine (Orn) unit and a lysine (Lys) unit; or
(ii) a trivalent amino acid unit comprising a —N(R)₂⁺— group, wherein the groups R are independently C1 to C6 alkyl, and are preferably methyl, and which unit can be derived from a trifunctional amino acid comprising a tertiary amino group selected from N-dialkylated 2,3-diaminopropionic acid (Dap), N-dialkylated 2,4-diaminobutanoic acid (Dab), N-dialkylated ornithine (Orn) and N-dialkylated lysine (Lys).
24. The compound or salt of any of items 1 to 23, wherein p is 1 and the hydrophilic modifying group R^{M1} is a group that can be derived from a hydrophilic amino acid which comprises, in addition to its —NH₂ and its —COOH functional group, a further hydrophilic functional group, preferably a hydrophilic functional group selected from —NH₂, —COOH, —NH— C(═NH)—NH₂, —C(═O)NH₂, —NH—C(═O)—NH₂, —OH and —P(═O)(OH)₂, or wherein p is 0.
25. The compound or salt of any of items 1 to 23, wherein p is 1 and the hydrophilic modifying group R^{M1} is an amino acid unit which is selected from a diaminopropionic acid (Dap) unit, 2,4-diaminobutanoic acid (Dab) unit, ornithine (Orn) unit and a lysine (Lys) unit, more preferably a diaminopropionic acid unit, or wherein p is 0.
26. The compound or salt of any of items 1 to 25, wherein q is 1 and the divalent linking group L^{D2} is a group of the formula (L-2), or q is 0:

-[A^{L2}]_{w}- (L-2)
wherein
A^{L2} is, independently for each occurrence if w is more than 1, an amino acid unit; and
w is 1 to 5, preferably 1 to 3, more preferably 1 or 2.
27. The compound or salt of item 26, wherein q is 1 and the amino acid unit(s) A^{L2} is (are) independently selected from a glycine unit and an alanine unit, or q is 0.
28. The compound or salt of any of items 1 to 27, which is a compound of formula (I.2) or a salt thereof:

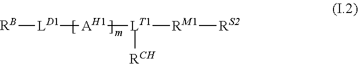

R^{B}, L^{D1}, A^{H1}, m L^{T1}, R^{CH}, and R^{M1} are defined as in the preceding items; and
R^{S2} is a group of the formula (S-3):

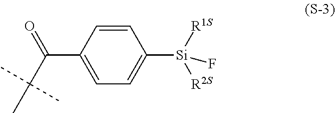

wherein
R^{1S} and R^{2S} are independently from each other a linear or branched C3 to C10 alkyl group, preferably R^{1S} and R^{2S} are selected from isopropyl and tert-butyl, and more preferably R^{1S} and R^{2S} are tert-butyl; and wherein the dashed line marks a bond which attaches the group to the remainder of the compound.
29. The compound or salt of any of items 1 to 27, which is a compound of formula (I.4) or (1.5) or a salt of these:

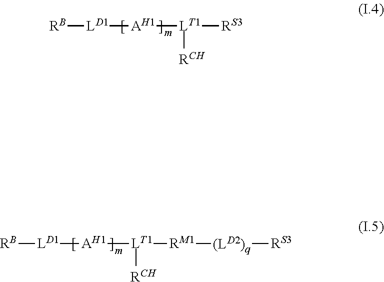

wherein
R^{B}, L^{D1}, A^{H1}, m, L^{T1}, R^{M1}, L^{D2}, q and R^{CH} are defined as in the preceding items; and
R^{S3} is a group of the formula (S-4):

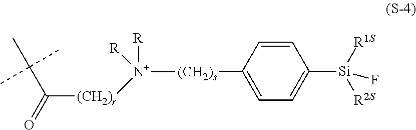

wherein
r is 1, 2 or 3, preferably 1, s is an integer of 1 to 6 and is preferably 1,
R is, independently, H or C1 to C6 alkyl, preferably H or C1 to C2 alkyl, and is more preferably methyl,
R^{1S} and R^{2S} are independently from each other a linear or branched C3 to C10 alkyl group, preferably R^{1S} and R^{2S} are selected from isopropyl and tert-butyl, and more preferably R^{1S} and R^{2S} are tert-butyl; and wherein the dashed line marks a bond which attaches the group to the remainder of the compound.
30. The compound or salt of any of items 1 to 27, which is a compound of formula (Ia) or a salt thereof:

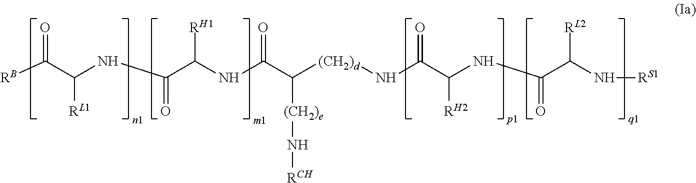

wherein
R^{B}, and R^{CH} are defined as in the preceding items;
n1 is 2 to 5, preferably 2 or 3;
R^{L1} is selected, independently for each occurrence from H, a C1-C4 alkyl group, —(CH₂)ᵥ—NH—C(═NH)—NH₂, —(CH₂)ᵥ—C(═O)NH₂, —(CH₂)ᵥ—NH—C(═O)—NH₂, and —(CH₂)ᵥ—OH wherein v is 1 to 4;
m1 is 2 to 5, more preferably 2 or 3;
R^{H} is selected, independently for each occurrence, from —(CH₂)ᵥ—NH₂, —(CH₂)ᵥ—COOH, —(CH₂)ᵥ—NH—C(═NH)—NH₂, —(CH₂)ᵥ—C(═O)NH₂, —(CH₂)ᵥ—NH—C(═O)—NH₂, —(CH₂)ᵥ—OH and —(CH₂)ᵥ—P(═O)(OH)₂, wherein v is 1 to 4;
d is an integer of 0 to 4, preferably 0 or 1;
e is an integer of 0 to 4, preferably 0 or 1; and more preferably one of d and e is 1, and the other one is 0;
p1 is 0 or 1;
R^{H2} is selected from —(CH₂)ᵥ—NH₂, —(CH₂)—COOH, —(CH₂)ᵥ—NH—C(═NH)—NH₂, —(CH₂)ᵥ—C(═O)NH₂, —(CH₂)ᵥ—NH—C(═O)—NH₂, —(CH₂)ᵥ—OH and —(CH₂)ᵥ—P(═O)(OH)₂ wherein v is 1 to 4;
q1 is an integer of 0 to 2;
R^{L2} is selected from H and CH₃, and
if p1 is 1, R^{S1} is selected from a group of formula (S-3) and a group of formula (S-4)

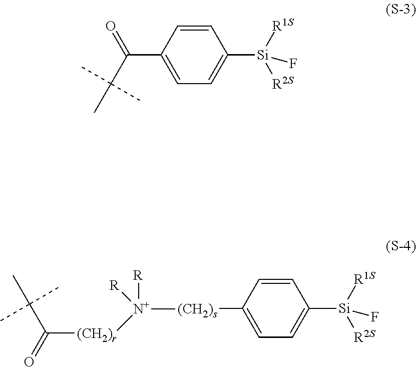

wherein
r is 1, 2 or 3, preferably 1, s is an integer of 1 to 6 and is preferably 1, R is, independently, H or C1 to C6 alkyl, preferably H or C1 to C2 alkyl, and is more preferably methyl, and
R^{1S} and R^{2S} are independently from each other a linear or branched C3 to C10 alkyl group, preferably R^{1S} and R^{2S} are selected from isopropyl and tert-butyl, and more preferably R^{1S} and R^{2S} are tert-butyl; and wherein the dashed line marks a bond which attaches the group to the remainder of the compound; and
if p1 is 0, R^{S1} is a group of formula (S-4) as defined above.
31. The compound or salt of item 30, which is a compound of formula (Ia.2) or a salt thereof:

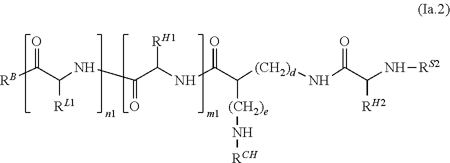

wherein
R^{B} and R^{CH} are defined as in the preceding items;
n1 is 2 to 5, preferably 2 or 3;
R^{L1} is selected, independently for each occurrence from H, a C1-C4 alkyl group, —(CH₂)ᵥ—NH—C(═NH)—NH₂, —(CH₂)ᵥ—C(═O)NH₂, —(CH₂)ᵥ—NH—C(═O)—NH₂, and —(CH₂)ᵥ—OH wherein v is 1 to 4;
m1 is 2 to 5, more preferably 2 or 3;
R^{H1} is selected, independently for each occurrence, from —(CH₂)ᵥ—NH₂, —(CH₂)ᵥ—COOH, —(CH₂)ᵥ—NH—C(═NH)—NH₂, —(CH₂)ᵥ—C(═O)NH₂, —(CH₂)ᵥ—NH—C(═O)— NH₂, —(CH₂)ᵥ—OH and —(CH₂)ᵥ—P(═O)(OH)₂ wherein v is 1 to 4;
d is an integer of 0 to 4;
e is an integer of 0 to 4; and preferably one of d and e is 0 and the other one is an integer of 1 to 4, more preferably the other one is 1;
R^{H2} is selected from —(CH₂)ᵥ—NH₂, —(CH₂)ᵥ—COOH, —(CH₂)ᵥ—NH—C(═NH)—NH₂, —(CH₂)ᵥ—C(═O)NH₂, —(CH₂)ᵥ—NH—C(═O)—NH₂, —(CH₂)ᵥ—OH and —(CH₂)ᵥ—P(═O)(OH)₂ wherein v is 1 to 4; and
R^{S2} is a group of the formula (S-3):

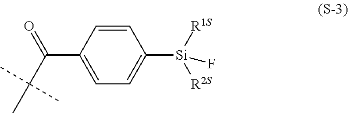

wherein
R^{1S} and R^{2S} are independently from each other a linear or branched C3 to C10 alkyl group, preferably R^{1S} and R^{2S} are selected from isopropyl and tert-butyl, and more preferably R^{1S} and R^{2S} are tert-butyl, and wherein the dashed line marks a bond which attaches the group to the remainder of the compound.
32. The compound or salt of item 30, which is a compound of formula (Ia.4) or a salt thereof or a compound of formula (Ia.5) or a salt thereof:

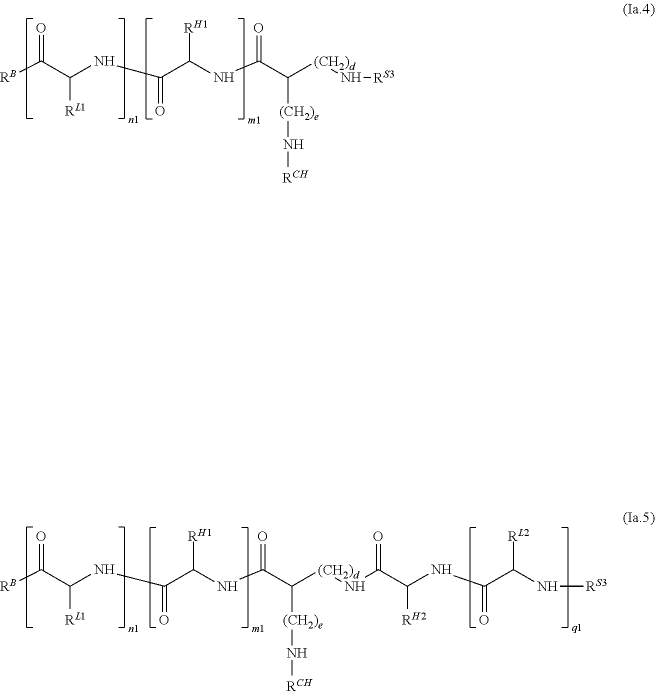

wherein
R^{B} and R^{CH} are defined as in the preceding items;
n1 is 2 to 5, preferably 2 or 3;
R^{L1} is selected, independently for each occurrence from H, a C1-C4 alkyl group, —(CH₂)ᵥ—NH—C(═NH)—NH₂, —(CH₂)ᵥ—C(═O)NH₂, —(CH₂)ᵥ—NH—C(═O)—NH₂, and —(CH₂)ᵥ—OH wherein v is 1 to 4;
m1 is 2 to 5, more preferably 2 or 3;
R^{H1} is selected, independently for each occurrence, from —(CH₂)ᵥ—NH₂, —(CH₂)ᵥ—COOH, —(CH₂)ᵥ—NH—C(═NH)—NH₂, —(CH₂)ᵥ—C(═O)NH₂, —(CH₂)ᵥ—NH—C(═O)—NH₂, —(CH₂)ᵥ—OH and —(CH₂)ᵥ—P(═O)(OH)₂ wherein v is 1 to 4;
d is an integer of 0 to 4;
e is an integer of 0 to 4; and preferably one of d and e is 0 and the other one is an integer of 1 to 4, more preferably the other one is 1;
R^{H2} is selected from —(CH₂)ᵥ—NH₂, —(CH₂)ᵥ—COOH, —(CH₂)ᵥ—NH—C(═NH)—NH₂, —(CH₂)˜—C(═O)NH₂, —(CH₂)ᵥ—NH—C(═O)—NH₂, —(CH₂)ᵥ—OH and —(CH₂)ᵥ—P(═O)(OH)₂ wherein v is 1 to 4;
q1 is 0 an integer of 0 to 2;
R^{L2} is selected from H and CH₃, and
R^{S3} is a group of the formula (S-4):

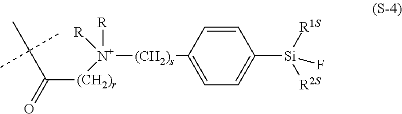

wherein
r is 1, 2 or 3, preferably 1, s is an integer of 1 to 6 and is preferably 1,
R is, independently, H or C1 to C6 alkyl, preferably H or C1 to C2 alkyl, and is more preferably methyl, and
R^{1S} and R^{2S} are independently from each other a linear or branched C3 to C10 alkyl group, preferably R^{1S} and R^{2S} are selected from isopropyl and tert-butyl, and more preferably R^{1S} and R^{2S} are tert-butyl; and wherein the dashed line attaches the group to the remainder of the compound.
33. The compound or salt of any of items 30 to 33, wherein R^{H1} is selected, independently for each occurrence, from —(CH₂)ᵥ—NH₂, —(CH₂)ᵥ—COOH, —(CH₂), —NH—C(═NH)—NH₂, —(CH₂)ᵥ—C(═O)NH₂, and —(CH₂)ᵥ—NH—C(═O)—NH₂, wherein v is 1 to 4.
34. The compound or salt of any of items 1 to 33, wherein the chelating group contains a chelated radioactive cation and the silicon-fluoride acceptor group is not labeled with ¹⁸F.
35. The compound or salt of item 34, wherein the chelated radioactive cation is a cation of 177Lu or a cation of ⁶⁸Ga.
36. The compound or salt of any of items 1 to 33, wherein the silicon-fluoride acceptor group is labeled with ¹⁸F and the chelating group contains a chelated non-radioactive cation or is free of a chelated cation.
37. A pharmaceutical composition comprising or consisting of one or more compounds or salts of any of items 1 to 36.
38. The compound or salt of any of items 1 to 36 for use as a medicament.
39. The compound or salt of any of items 1 to 36 or the pharmaceutical composition of item 37 for use in the treatment or prevention of cancer, wherein the cancer is preferably neuroendocrine tumor.
40. A diagnostic composition comprising or consisting of a compound or salt of any of items 1 to 36.
41. The compound or salt of any of items 1 to 36 or the diagnostic composition of item 40 for use in a method of diagnosis in vivo of a disease or disorder.
42. The compound or salt or the diagnostic composition for use of item 41, wherein the disease or disorder is cancer, and wherein the cancer is preferably neuroendocrine tumor.
43. The compound or salt or the diagnostic composition for use of item 41 or 42, wherein the method of diagnosis involves nuclear diagnostic imaging, wherein the nuclear diagnostic imaging is preferably positron emission tomography or single photon emission computerised tomography imaging.

In this specification, a number of documents including not only scientific journal articles but also patent applications and manufacturer's manuals are cited (cf, e.g., the list of references below in this respect). The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### Abbreviations

2-CTC—2-Chlorotrityl chloride
Acm—Acetamidomethyl
AR42J—Rat exocrine pancreatic tumour
Arg—Arginine
Asp—Aspartic acid
BA—Benzoic acid
Boc—tert-Butoxycarbonyl
BSA—Bovine Serum Albumine
CHO—Chinese hamster ovary
CT—Computed tomography
Cys—Cysteine
d—D-Isomer, Doublet
DCM—Dichloromethane
Dde—2-Acetyldimedone
DIPEA—N,N-Diisopropylethylamine
DMEM/F-12—Dulbecco's Modified Eagle Medium/Nutrient Mixture F-12
DMF—Dimethylformamide
DMSO—Dimethyl sulfoxide
eq—Equivalent
ESI-MS—Electrospray ionization mass spectrometry
FCS—Fetal Calf Serum, Fetal Calf Serum
Fmoc—Fluorenylmethyloxycarbonyl
GBq—Gigabecquerels
Gly—Glycine
GP—General Procedure
HATU—Hexafluorophosphate Azabenzotriazole Tetramethyl Uronium
HBSS—Hank's Balanced Salt Solution
HBSS-B—HBSS with 1% BSA
HEPES—4-(2-hydroxyethyl)-1-piperazineethanesulfonic acid
HFIP—Hexafluoro-2-propanol
HOAt—1-Hydroxy-7-azabenzotriazole
HOBt—Hydroxybenzotriazole
HPLC—High Performance Liquid Chromatography
HSA—Human Serum Albumine
IC₅₀—half maximal inhibitory concentration
J—Spin-spin coupling
Kₐᵥ—Partition coefficient
I—L-Isomer, Loading density
Lys—Lysine
M—Molecular weight
m/z—Mass-to-charge ratio
MBq—Megabecquerels
m_{exact}—Calculated exact mass
m_{found}—Experimentally determined mass
MHz—Megahertz
n—Number of experiment repetitions
nm—Nanomolar, Nanometer
NMP—N-Methylpyrrolidone
OI—Optical imaging
Orn—Ornithine
p—Para
PBS—Phosphate Buffered Saline
PCC—Pyridinium chlorochromate
PEG—Polyethylene glycol
PET—Positron emissuin tomography
PG—Protecting group
Phe—Phenylalanine
PLL—Poly-L-lysine
Pma—Phosphonomethylalanine
ppm—Parts per million
Rf—Retention factor
RP—Reversed Phase
RT—Room temperature
s—Singlet
SiFA—Silicon Fluoride Acceptor
SPECT—Single photon emission computed tomography
SST₂—Somatostatin receptor type 2
TATE—Tyr³-Octreotate
TBDMS—tert-Butyldimethylsilyl
TBq—Terrabecquerels
TBTU—Hexafluorophosphate Benzotriazole Tetramethyl Uronium
tBu—tert-Butyl
TFA—Trifluoroacetic acid
THF—Tetrahydrofuran
Thr—Threonine
TIPS—Triisopropylsilane
TLC—Thin layer chromatography
TOC—Octreotid
t_{R}—Retention time
TRIS—Tris(hydroxymethyl)aminomethane
Trp—Tryptophan
Tyr—Tyrosine
UV/VIS—Ultraviolet/Visible
v/v—Percentage by volume
V₀—Void volume
Vₑ—Elution volume
Vol-%—Percentage by volume
Xaa—Amino acid
γ—Gamma
δ—Chemical shift in ppm

### EXAMPLES

### I. Materials and Methods

### 1. General Methods

1.1 Reagents, Solvents and radioactive Isotopes

The reagents and solvents were used without further purification. The used solvents were purchased from VWR International (Buchsal, Germany) or Sigma Aldrich (Munich, Germany). Water for the HPLC-solvents was obtained from the in-house Millipore-system from Thermo Fischer Scientific Inc. (Waltham MA, USA), while Tracepure water derived from the company Merck Millipore (Darmstadt, Germany). Amino acids were purchased from IRIS Biotech GmbH (Marktredewitz, Germany), Sigma-Aldrich (Munich, Germany), Carbolution Chemicals GmbH (St. Ingbert, Germany), Merck Millipore (Darmstadt, Germany). Resins were purchased from IRIS Biotech GmbH (Marktredewitz, Germany) or CEM (Matthews, USA). Coupling reagents and other chemicals derive from Sigma-Aldrich (Munich, Germany), Molekula GmbH (Garching, Germany), CEM (Matthews, USA) and Macrocyclics Inc. (Dallas, USA). Chemicals for synthesis were purchased from the company Sigma-Aldrich (Munich, Germany) and Merck KGaA (Darmstadt, Germany). The used chelators derived from CheMatech (Dijon, France). Radioactive labeling with ¹²⁵I was performed with a [¹²⁵I]Nal solution in 40 mM NaOH (74 TBq/mmol) from HARTMANN ANALYTIC GmbH (Braunschweig, Deutschland). ¹⁸F for radioactive labeling was received from Klinikum Rechts der Isar (Technische Universitst München, München, Deutschland). Biochemicals as cell mediums, PBS and Trypsin were bought from Biochrom GmbH (Berlin) and Sigma-Aldrich (Munchen, Deutschland)

### 1.2 Instruments and Analytics

### 1.2.1 RP-HPLC System

Reaction- and quality-controls were conducted via analytical RP-HPLC. For this purpose, a linear gradient of MeCN (0.1% TFA, 2% H₂O; v/v) in H₂O (0.1% TFA; v/v) was run. The respective gradients can be taken from the synthesis instructions. The detection was conducted via an UV/VIS-detector at λ=220 nm or λ=254 nm. All RP-HPLC chromatograms were evaluated using the LabSolutions Software from the Shimadzu Corp. (Kyoto, Japan). For the analytical studies following systems have been used:

Shimadzu Corp. (Kyoto, Japan): Consisting of two LC-20AD gradient-pumps, a CBM-20A communication module, a CTO-20A column oven, a SPD-20A UV/VIS-detector and a MultoKrom 100-5 C₁₈-column (5 μm, 125×4.6 mm, CS Chromatographie GmbH, Langerwehe, Germany) with a flow of 1 mL/min.

The complete purification of intermediate and end products was performed via semi-preparative RP-HPLC. Here again a linear gradient of MeCN (0.1% TFA, 5% H₂O; v/v) in H₂O (0.1% TFA; v/v) was run. The respective gradients can be taken from the synthesis instructions. The detection was conducted via an UV/VIS-detector at λ=220 nm or λ=254 nm. All RP-HPLC chromatograms were evaluated using the LabSolution Software from the Shimadzu Corp. (Kyoto, Japan). For preparative purification two systems of the Shimadzu Corp. (Kyoto, Japan) have been used:
Shimadzu Corp. (Kyoto, Japan): Consisting of two LC-20AP gradient-pumps, a DGU-20A degassing unit, a CBM-20A communication module, a CTO-20A column oven, a SPD-20A UV/VIS-detector and a MultoKrom 100-5 C₁₈-column (5 μm, 250×10 mm, CS Chromatographie GmbH, Langerwehe, Germany) with a flow of 5 mL/min.
Shimadzu Corp. (Kyoto, Japan): Consisting of two LC-20AT gradient-pumps, a DGU-20A degassing unit, a CBM-20A communication module, a SPD-20A UV/VIS-detector and a MultoKrom 100-5 C₁₈-column (5 μm, 250×10 mm, CS Chromatographie GmbH, Langerwehe, Germany) with a flow of 5 mL/min.
   1.2.2 ESI-MS Spectrocopy Mass spectrometry was performed using an Expression CMS Quadrupole device from the company Advion Inc. (Ithaca, USA).

### 1.2.3 Flash-Chromatography

Flash-chromatographic purifications were conducted at an Isolera™ Prime System from the company Biotage (Uppsala, Sweden) with a Biotage 09474 Rev. E Bio pump from the same company. For purification a linear gradient of MeCN (0.1% TFA, 2% H₂O; v/v) in H₂O (0.1% TFA; v/v) was run. Furthermore, a Biotage™ SNAP KP-C₁₈ cartridge (12 g cartridge material, pore diameter: 93 Å, surface: 382 m²/g from the company Biotage (Uppsala, Sweden) was used.

### 1.2.4 Lyophilisation

The lyophilization of intermediate- and end-products was conducted at an Alpha 1-2 lyophilization instrument from the company Christ (Osterode am Harz, Germany) using a RZ-2 vacuum-pump from Vacubrand GmbH. The substance to be dried was dissolved beforehand in H₂O and tBuOH (1/1; v/v) and the solution was frozen at −80° C.

### 1.2.5 NMR

¹H- and ¹³C-NMR spectra were recorded on a Bruker (Billerica, Massachusetts, USA) AVHD-300 or AVHD-400 at 300 K. ¹H and ¹³C chemical shifts are reported in ppm and referenced to the residual proton of the solvent. Deuterated solvents were obtained from Sigma Aldrich.

### 2. Organic Synthesis

### 2.1 Synthesis of SiFA-Br and SiFA-BA

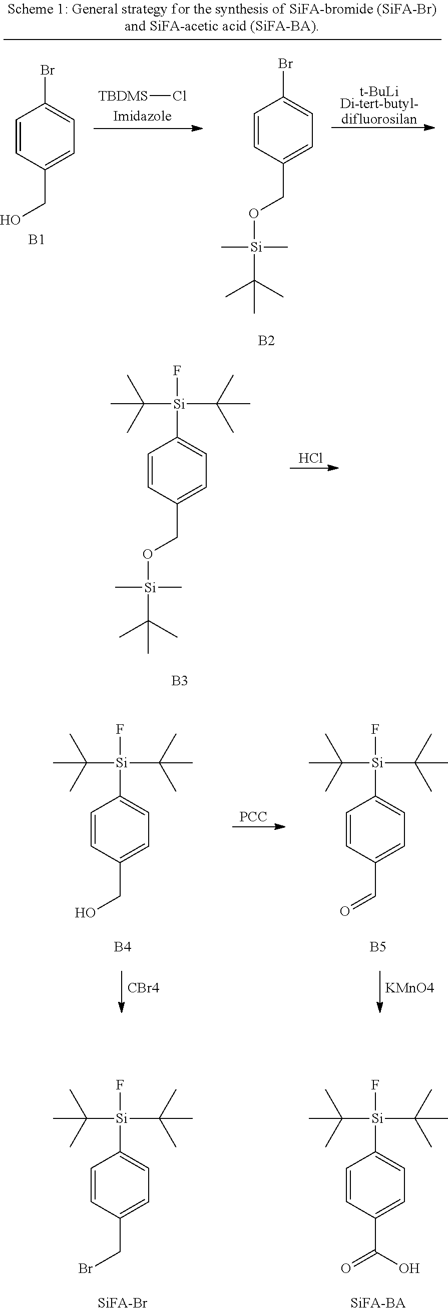

### ((4-Bromobenzyl)oxy)(tert-butyl)dimethylsilane (B2)

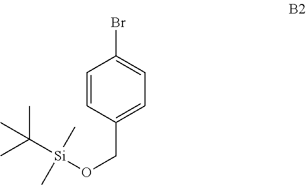 [SMILES: CC(C)(C)[Si](C)(C)OCc1ccc(Br)cc1]

In a round bottom flask, 4.68 g of 4-Bromobenzyl alcohol (B1, 25.0 mmol, 1.00 eq.) are dissolved and stirred in 70 mL dry DMF. 2.04 g of imidazole (30.0 mmol, 1.20 eq.) and 4.52 g TBDMS-chloride (30.0 mmol, 1.20 eq.) are added under stirring. The mixture is left for reaction for 20 h at room temperature. The reaction is then poured into 250 mL of ice-cold H₂O and the organic phase is extracted with Et₂O (5×50 mL). The combined organic phases are washed with a saturated aqueous solution of NaHCO₃ (100 mL), brine (100 mL) and dried over Na₂SO₄. The solvent is removed under reduced pressure and the crude product is purified via column chromatography (5% EtOAc in petroleum ether). After the solvents are removed under reduced pressure, 7.24 g of the product B2 (24.1 mmol, 96%) are yielded as a colorless oil. TLC (SiO₂, 5% EtOAc/petroleum ether): R_{f}=0.97 [UV]

¹H-NMR (300 MHz, CDCl₃): δ [ppm]=7.45 (d, ³J=8 Hz, 2H, H_{Ar}), 7.20 (d, ³J=8 Hz, 2H, H_{Ar}), 4.68 (s, 2H, Ar—CH₂), 0.94 (s, 9H, C—CH₃), 0.10 (s, 6H, Si—CH₃).

¹³C-NMR (75 MHz, CDCl₃): δ [ppm]=140.3 (s, Cᵢ), 130.1 (s, Cₘ), 127.5 (s, Cₒ) 120.4 (s, Cₒ), 64.2, (s, CH₂), 25.8 (s, C—CH₃), 18.2, (s, C—CH₃) 5.4 (s, Si—CH₃).

### Di-tert-butyl(4-(((tert-butyldimethylsilyl)oxy)methyl)phenyl)fluorosilane (B3)

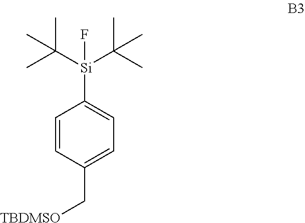 [SMILES: CC(C)(C)[Si](C)(C)OCc1ccc([Si](F)(C(C)(C)C)C(C)(C)C)cc1]

In an argon atmosphere, 7.24 g B2 (24.1 mmol, 1.00 eq.) are dissolved in 67 mL dry THF and cooled to −78° C. (dry ice and acetone). Over a period of 1.5 h, 32.6 mL of a 1.7 M solution of tBuLi in pentane (55.4 mmol, 2.30 eq.) are slowly dropped to the solution of B2 in THF. The mixture is left for stirring at −78° C. for an additional 30 min. In another round bottom flask, 5.00 g of di-tert-butyldifluorosilane (27.7 mmol, 1.10 eq.) are dissolved in 44 mL of dry THF and also cooled to −78° C. Over a period of 2 h and under constant stirring, the mixture of B2 and tBuLi in THF is slowly dropped to the solution of di-tert-butyldifluorosilane. The reaction is allowed to warm to room temperature and is left under stirring for another 15 h. Through addition of 120 mL of brine the reaction is terminated and the organic phase is separated. The aqueous phase is extracted with Et₂O (3×100 mL), the combined organic phases are dried over MgSO₄ and the solvents are removed under reduced pressure. The product B3 is yielded as a yellowish oil (9.14 g, 23.9 mmol, 99%).

¹³C-NMR (75 MHz, CDCl₃): δ [ppm]=143.0 (s, Cᵢ), 134.1 (d, ³J (⁹³C, ¹⁹F)=12 Hz, Cₘ), 132.0 (d, ²J (¹³C, ¹⁹F)=56 Hz, Cₚ), 125.3 (s, Cₒ), 65.0 (s, CH₂), 27.5 (s, CH₃), 26.8 (s, C—CH₃), 26.1 (s, CH₃), 20.4 (d, ²J (¹³C, ¹⁹F)=8 Hz, C—CH₃), 5.11 (s, Si—CH₃).

### (4-(Di-tert-butylfluorosilyl)phenyl)methanol (B4)

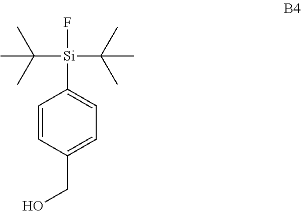 [SMILES: CC(C)(C)[Si](F)(c1ccc(CO)cc1)C(C)(C)C]

Compound B3 (9.14 g, 23.9 mmol, 1.00 eq.) is dissolved in 50 mL MeOH. After the addition of 3.00 mL of concentrated HCl (97.9 mmol, 4.10 eq.) the solution is left for reaction for 18 h at room temperature. The mixture is concentrated under reduced pressure, the precipitate is dissolved in 50 mL of Et₂O and the organic phase is washed with 50 mL of a saturated, aqueous solution of NaHCO₃. The aqueous phase is extracted with Et₂O (3×50 mL), the combined organic phases are combined and dried over MgSO₄. The solvents are removed under reduced pressure and the product B4 (5.90 g, 22.0 mmol, 92%) is yielded as a yellowish oil.

¹H-NMR (CDCl₃): δ [ppm]=7.61 (d, 2H, ³J=8 Hz, H_{Ar}), 7.38 (d, 2H, ³J=8 Hz, H_{Ar}), 4.72 (s, 2H, Ar—CH₂), 1.06 (s, 18H, C—CH₃).

¹³C-NMR (CDCl₃): δ [ppm]=142.3 (s, Cᵢ), 134.4 (d, ³J(¹³C, ¹⁹F)=12 Hz, Cₘ), 133.1 (d, ²J(¹³C, ¹⁹F)=56 Hz, Cₚ), 125.6 (s, Cₒ), 65.4 (s, CH₂), 27.4 (s, CH₃), 20.4 (d, ²J (¹³C, ¹⁹F)=8 Hz, C—CH₃).

HPLC (50-100% B in 15 min) t_{R}=10.7 min

### 4-(Di-tert-butylfluorosilyl)benzaldehyde (B5)

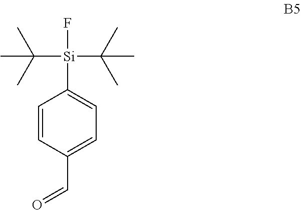 [SMILES: CC(C)(C)[Si](F)(c1ccc(C=O)cc1)C(C)(C)C]

The Alcohol B4 (5.90 g, 55.0 mmol, 2.5 eq.) is dissolved in 60 mL dry DCM and slowly dropped to an ice-cold solution of 11.9 g PCC (55.0 mmol, 2.5 eq.) in 180 mL DCM. The solution is stirred for 30 min at 0° C. and another 4 h at room temperature. The reaction is terminated through the addition of 60 mL of Et₂O and the supernatant is decanted. The insoluble, black residue is washed thoroughly with Et₂O and the combined organic phases are filtered over a short silica plug. Solvent removal under reduced pressure is followed by purification via column chromatography (2.5% EtOAc in petroleum ether). Product B5 is yielded as yellowish oil, slowly crystallizing to a yellowish solid (2.22 g, 8.33 mmol, 36%).

¹H-NMR (CDCl₃): δ [ppm]=10.1 (s, 1H, CHO), 7.88 (d, ³J=6 Hz, 2H, H_{Ar}), 7.79 (d, ³J=9 Hz, 2H, H_{Ar}), 1.07 (s, 18H, C—CH₃).

¹³C-NMR (CDCl₃): 5 [ppm]=192.7 (s, CHO), 142.5 (s, Cᵢ), 137.2 (s, Cₚ), 134.7 (d, ³J (¹³C, ¹⁹F)=12 Hz, Cₘ), 128.6 (s, Cₒ), 27.4 (s, CH₃), 20.5 (s, C—CH₃).

¹⁸F-NMR (CDCl₃)=δ [ppm]=188.4 (s, F).

HPLC (50-100% B in 15 min) t_{R}=15.9 min.

### 4-(Di-tert-butylfluorosilyl)benzoic acid (SiFA-BA)

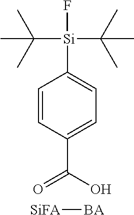

The aldehyde B5 (1.0 eq.) is dissolved in tBuOH (23 mL/g starting material), DCM (2.5 mL/g starting material) and NaH₂PO₄xH₂O (1.25 M, pH=4.0-4.5, 15 mL/g educt) and KMnO_{4aq.} (1 M, 23 mL/g starting material) is added. After stirring for 25 min, the mixture is cooled to 5° C. KMnO₄ (1.0 eq.) is added and the reaction quenched shortly afterwards by addition of a saturated, aqueous solution of NaHCO₃. The mixture is dried over MgSO₄ and the solvent evaporated under reduced pressure. The crude product is purified by recrystallization from Et₂O/n-hexane (1/3 v/v) and SiFA-BA is afforded as a colorless solid (60% yield).

¹H-NMR (300 MHz, CDCl₃): δ [ppm]=8.10 (d, ³J=8.1 Hz, 2H, Hₘ), 7.74 (d, ³J=8.1 Hz, 2H, Hₒ), 1.07 (s, 18H, CCH₃).

¹³C-NMR (75 MHz, CDCl₃): δ [ppm]=171.0 (s, COOH), 141.4 (d, Cₚ), 134.2 (d, Cₘ), 129.0 (d, C_{i;o}), 27.4 (s, CCH₃), 20.4 (d, CCH₃).

RP-HPLC (50-100% in 15 min) t_{R}=8.4 min.

### (4-(Bromomethyl)phenyl)di-tert-butylfluorosilane (SiFA-Br)

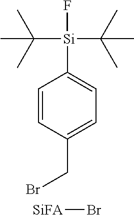

To a 0° C. cooled solution of B4 (3.08 g, 11.5 mmol, 1.0 eq.) and tetrabromomethane (4.18 g, 12.6 mmol, 1.1 eq.) in 100 mL DCM, triphenylphosphine (3.30 g, 12.6 mmol, 1.1 eq.) was added over a period of 30 min in small portions. The solution was stirred for 2 h at room temperature. Solvents were removed in vacuo and the residue was washed with cold n-hexane (3×50 mL). A white precipitate was removed by filtration and the solution was concentrated in vacuo. Purification was conducted by flash column chromatography (silica, 5% EtOAc in petrol, v/v). Compound SiFA-Br was isolated as a colorless oil (3.06 g, 9.20 mmol, 80%).

RP-HPLC (50 to 100% B in 15 min): t_{R}=9.2 min, K′=3.73.

¹H-NMR (400 MHz, CDCl₃): δ [ppm]=7.58 (2H, d, C₆H₄), 7.40 (2H, d, C₆H₄), 4.49 (2H, s, CH₂OSi), 1.05 (18H, s, Si(tBu)₂).

### 3. Peptide Synthesis

### 3.1 General Procedures (GP)

### 3.1.1 General Procedures for Solid-Phase-Peptide-Synthesis (SPPS)

By means of the solid phase peptide synthesis via Fmoc-strategy, peptides are synthesized on a resin, whereby Fmoc is used as temporary protective group. The resin is loaded with a Fmoc-protected amino acid (Xaa), subsequently it is Fmoc-deprotected and brought to reaction with the next amino acid. After completion of the aspired peptide, it is split off the resin. Before peptide elongation, cleavage of protecting groups or any other type of chemical modification, the already loaded resin has to be swollen for 30 min in a suitable solvent (DMF, DCM, NMP). After each reaction step, the resin has to be washed thoroughly, with the solvent used for the reaction. If no further reaction step is conducted, the resin has to be washed with DCM and dried in vacuo.

### GP1: 2-CTC-Resin Loading

Loading of the 2-Chlorotritylchloride resin (2-CTC) with a Fmoc-protected amino acid (Fmoc-Xaa-OH) is carried out by stirring a suspension of the 2-CTC-resin (1.60 mmol/g and Fmoc-Xaa-OH (0.70 eq.) in DMF with DIPEA (4.5 eq.) at room temperature for 4 h. Remaining tritylchloride is capped by addition of methanol (2 mL/g resin) for 15 min. Subsequently the resin is filtered and washed with DMF (5×5 mL/g resin), methanol (3×5 mL/g resin) and dried in vacuo. Final loading l of Fmoc-Xaa-OH is determined by the following equation:

$$l [ \frac{m mol}{g} ] = \frac{( {m}_{2} - {m}_{1} ) \times 1000}{( {M}_{W} - {M}_{HCl} ) {m}_{2}} \begin{matrix}{m}_{2} = mass of loaded resin [ g ] \\ {m}_{1} = mass of unloaded resin [ g ] \\ {M}_{W} = molecular weight of Xaa [ g / mol ] \\ {M}_{HCl} = molecular weight of HCl [ g / mol ]\end{matrix}$$

### GP2: On-Resin Peptide Formation

The respective side-chain protected Fmoc-Xaa-OH (1.5 eq.) is dissolved in DMF (8 mL/g resin) and pre-activated by adding TBTU or HATU (1.5 eq.), HOBt or HOAt (1.5 eq.) and DIPEA (4.5 eq.) or 2,4,6-collidine (5.5 eq.). The solution is left for pre-activation for 10 min, followed by addition to the resin-bound peptide NH₂-(Xaa)ₓ-2-CT and shaken for 2 h at room temperature. In case of amino acids, sensible to racemization (e.g. Dap, Cys), the pre-activation time is strictly limited to 2 min and 2,4,6-collidine is used as base. Subsequently, the resin is washed with DMF (6×5 mL/g resin) and after Fmoc-deprotection the next amino acid is coupled analogously. The coupling of non-aminoacidic building blocks (SiFA-BA etc.) is carried out following the same protocol.

### GP3: On-Resin Chelator Conjugation

The chelator (1.5 eq.) (e.g. DOTA(tBu)₃, R/S-DOTAGA(tBu)₄) is dissolved in DMF (8 mL/g resin) and pre-activated by adding HATU (1.5 eq.), HOAT (1.5 eq.) and DIPEA (4.5 eq.). After pre-activation for 10 minutes, the solution is added to the resin bound peptide H₂N-(Xaa)ₓ-2-CT and shaken for 6 to 18 h. Completeness of the reaction has to be confirmed by RP-HPLC and ESI-MS. Subsequently, the resin is washed with DMF (6×5 mL/g resin).

### GP4: On-Resin Fmoc-Deprotection

The resin-bound Fmoc-peptide is treated with 20% piperidine in DMF (v/v, 8 mL/g resin) for 5 min and subsequently for 15 min. Afterwards, the resin is washed thoroughly with DMF (8×5 mL/g resin).

### GP5: On-Resin Dde-Deprotection

Dde-deprotection is performed by adding a solution of imidazole (0.46 g), hydroxylamine hydrochloride (0.63 g) in NMP (2.5 mL) and DCM (0.5 mL) for 3 h at room temperature. After deprotection the resin is washed with DMF (6×5 mL/g resin).

GP6: Peptide Cleavage from the Resin (Preservation of Acid labile Groups)

The resin-bound peptide is cleaved and dissolved in a mixture of HFIP/DCM (v/v; 4/1, 8 mL/g resin) and shaken for 45 min. The solution containing the fully protected peptide is filtered off and the resin is treated with another portion of the cleavage solution for 45 min. Both fractions are combined and the solvents are removed in vacuo. After lyophilisation in tBuOH/H₂O, the crude, fully protected peptide is obtained.

GP7: Peptide Cleavage from the Resin (Deprotection of all Acid Labile Groups)

The fully protected resin-bound peptide is dissolved in a mixture of TFA/TIPS/H₂O (v/v/v; 90/2.5/7.5) and shaken for 45 min. The solution is filtered off and the resin is treated in the same way for another 45 min. Complete deprotection is achieved by combining both filtrates and incubation at 40° C. for 1 h and at room temperature for 2 h (no chelator with tBu-protecting groups apparent the peptide) or 12 h (chelator with tBu-protecting group e.g. DOTA(tBu)₃ apparent in the peptide). After concentration under a stream of nitrogen, the crude product is dissolved in a mixture of tert-butanol and water followed by subsequent lyophilisation to obtain the crude peptide.

### GP8: Reaction Control Via Test-Cleavage

To verify the completeness of an amino acid coupling or any other kind of chemical modification, a small portion of resin is transferred into a 1.5 mL reaction tube and treated with the under GP6 and GP7 described solutions to either obtain the protected (in the following described as mild test cleavage) or unprotected (in the following described as harsh test cleavage) peptide. After concentration under a stream of nitrogen the remains are dissolved in RP-HPLC solvent and therefore suitable for RP-HPLC and ESI-MS investigation.

### 3.1.2 General Procedures for Synthesis in Solution

GP9: Formation of [^{nat}Ga] Complexes

The ligand is dissolved in DMSO at a 2 mM concentration. Since the solvents for the purification via semi-preparative RP-HPLC are acidified with TFA, the formation of TFA salts is assumed and included in the molecular weight. For complexation, a defined volume of the peptide solution is mixed with 1.5 eq. of [^{nat}Ga]Ga(NO₃)₃ in H₂O (20 mM). DMSO is added to generate a final concentration of 1 mm. The solution is left at 70° C. for 40 min and the stoichiometric conversion of the chelator is confirmed by RP-HPLC and ESI-MS.

GP10: Formation of [^{nat}Lu] Complexes

The ligand is dissolved in DMSO at a 2 mM concentration. Since the solvents for the purification via semi-preparative RP-HPLC are acidified with TFA, the formation of TFA salts is assumed and included in the molecular weight. For complexation, a defined volume of the peptide solution is mixed with 1.5 eq. of [^{nat}Lu]LuCl₃ in H₂O (20 mM). DMSO is added to generate a final concentration of 1 mM. The solution is left at 70° C. for 40 min and the stoichiometric conversion of the chelator is confirmed by RP-HPLC and ESI-MS.

GP11: Formation of [^{nat}Pb] Complexes

The ligand is dissolved in DMSO at a 2 mM concentration. Since the solvents for the purification via semi-preparative RP-HPLC are acidified with TFA, the formation of TFA salts is assumed and included in the molecular weight. For complexation, a defined volume of the peptide solution is mixed with 1.1 eq. of [^{nat}Pb]PbCl₂ in H₂O (10 mM). DMSO is added to generate a final concentration of 1 mm. The solution is left at 70° C. for 40 min and the stoichiometric conversion of the chelator is confirmed by RP-HPLC and ESI-MS.

### 3.2 Synthesis of Building Blocks

### Synthesis of SiFA-BA-D-Asp-OtBu (B6)

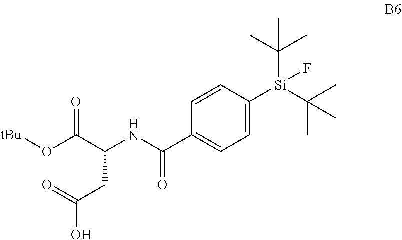 [SMILES: CC(C)(C)OC(=O)[C@@H](CC(=O)O)NC(=O)c1ccc([Si](F)(C(C)(C)C)C(C)(C)C)cc1]

The on-resin synthesis of SiFA-BA-D-Asp-OtBu (B6) is carried out applying general procedures GP1, GP2, GP4 and GP6. Briefly, Fmoc-D-Asp-OtBu (0.7 eq.) is loaded onto 2-CTC resin (1.0 eq.; loading capacity 1.6 mmol/g). After Fmoc-deprotection, SiFA-BA (1.5 eq.) is conjugated, applying HOAt (1.5 eq.), TBTU (1.5 eq.) and 2,4,6-collidine (5.5 eq.). To separate the peptide from the resin under preservation of the tBu-protecting group, the resin is treated with HFIP/DCM (v/v; 4/1, 8 mL/g resin) as described in GP6. After final lyophilisation, crude product B6 is yielded as colorless solid. Correct product formation is confirmed by RP-HPLC and ESI-MS.

RP-HPLC (10-90% in 15 min): t_{R}=17.1 min. ESI-MS: m_{exact} (C₁₉H₂₈FNO₅Si): 397.2; m_{found}: m/z=398.2 [M+H]⁺.

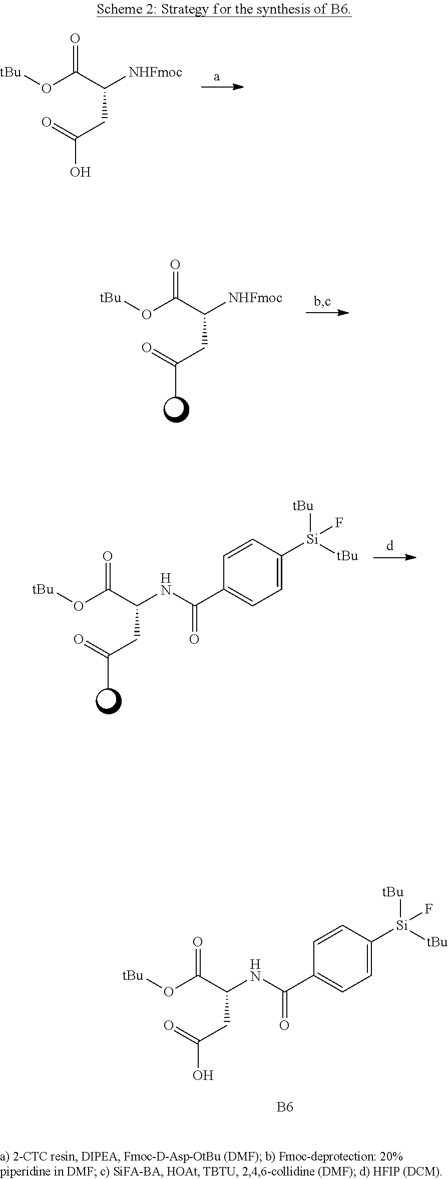

### Synthesis of Boc-D-Orn(Boc)-D-Orn(Boc)-OH (17)

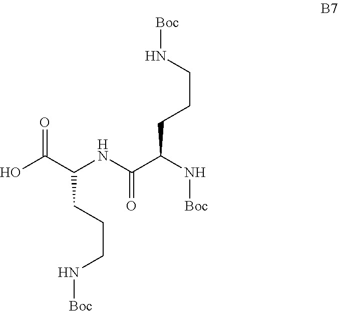

The on-resin synthesis of Boc-D-Orn(Boc)-D-Orn(Boc)-OH (B7) is carried out applying general procedures GP1, GP2, GP4 and GP6. Briefly, Fmoc-D-Orn(Boc)-OH (0.7 eq.) is loaded onto 2-CTC resin (1.0 eq.; loading capacity 1.6 mmol/g). After Fmoc-deprotection, Boc-D-Orn(Boc)-OH (1.5 eq.) is conjugated, applying HOAt (1.5 eq.), TBTU (1.5 eq.) and 2,4,6-collidine (5.5 eq.). Mild peptide cleavage from the resin is carried out according to GP6. After final lyophilisation, crude product B7 is yielded as colorless solid. Correct product formation is confirmed by RP-HPLC and ESI-MS.

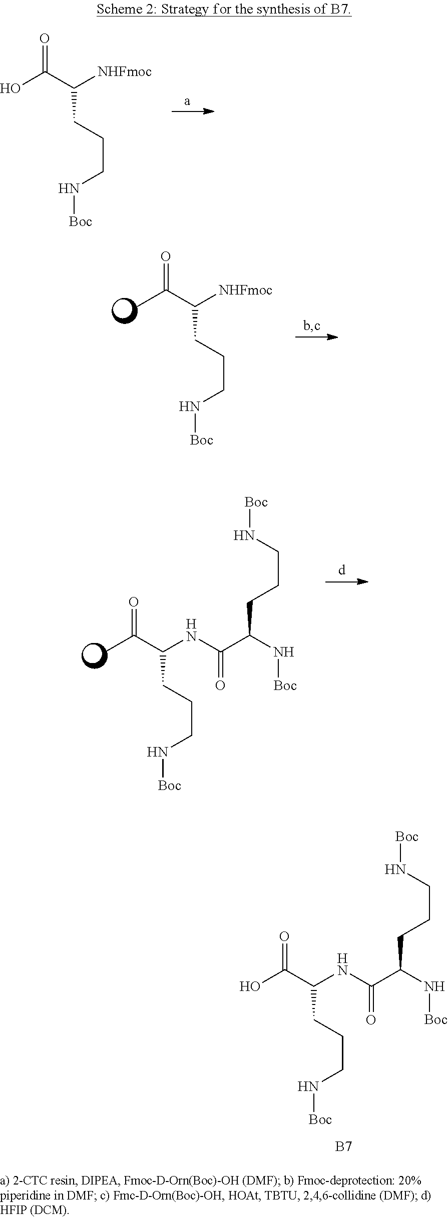

### 3.3 Synthesis of the Binding Motifs

### 3.3.1 Tyr³-Octreotate (TATE or X1)

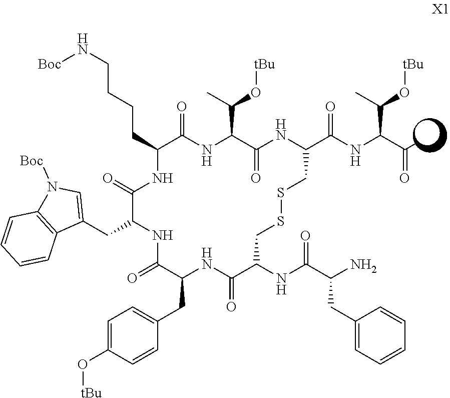

### Peptide Sequence

The on-resin synthesis of fully protected TATE is carried out analogously to published protocols applying general procedures GP1, GP2 and GP4 [19, 25]. Briefly, Fmoc-L-Thr(tBu)-OH (0.7 eq.) is loaded to 2-CTC resin (1.0 eq.; loading capacity 1.6 mmol/g), followed by Fmoc-deprotection and subsequent coupling of Fmoc-L-Cys(Acm)-OH, Fmoc-L-Thr(tBu)-OH, Fmoc-L-Lys(Boc)-OH, Fmoc-D-Trp(Boc)-OH, Fmoc-L-Tyr(tBu)-OH, Fmoc-L-Cys(Acm)-OH and Fmoc-D-Phe-OH. For the conjugation of all amino acids the following protocol is applied: Fmoc-Xaa-OH (1.5 eq.), HOAt (1.5 eq.), TBTU (1.5 eq.) and 2,4,6-collidine (5.5 eq.). The Fmoc protecting group of the last amino acid remains until the disulfide bridge is formed.

### Disulfide-Bridge Formation [19, 25-27]

Fmoc-D-Phe-L-Cys(Acm)-L-Tyr(tBu)-D-Trp(Boc)-L-Lys(Boc)-L-Thr(tBu)-L-Cys(Acm)-L-Thr(tBu)-2-CT (1.0 eq.) is treated with TI(TFA)₃ (4.0 eq.) in DMF for 45 min. After discarding the first solution the procedure is repeated with a new portion of TI(TFA)₃, for another 45 min. The resin is washed with DMF (6×5 mL/g resin) and completeness of cyclization is verified by RP-HPLC and ESI-MS (test cleavage: TFA/H₂O/TIPS 90/2.5/7.5) confirming the synthesis of Fmoc-D-Phe-cyco[L-Cys-L-Tyr(tBu)-D-Trp(Boc)-L-Lys(Boc)-L-Thr(tBu)-L-Cys]-L-Thr(tBu)-2-CT. Finally, the peptide is Fmoc-deprotected leading to H-TATE(PG)-2-CT (X1).

RP-HPLC (10-90% in 15 min): t_{R}=10.8 min.

ESI-MS: m_{exact} (C₆₄H₇₄N₁₀O₁₄S₂): 1270.5; m_{found}: m/z=1273.4 [M+H]⁺.

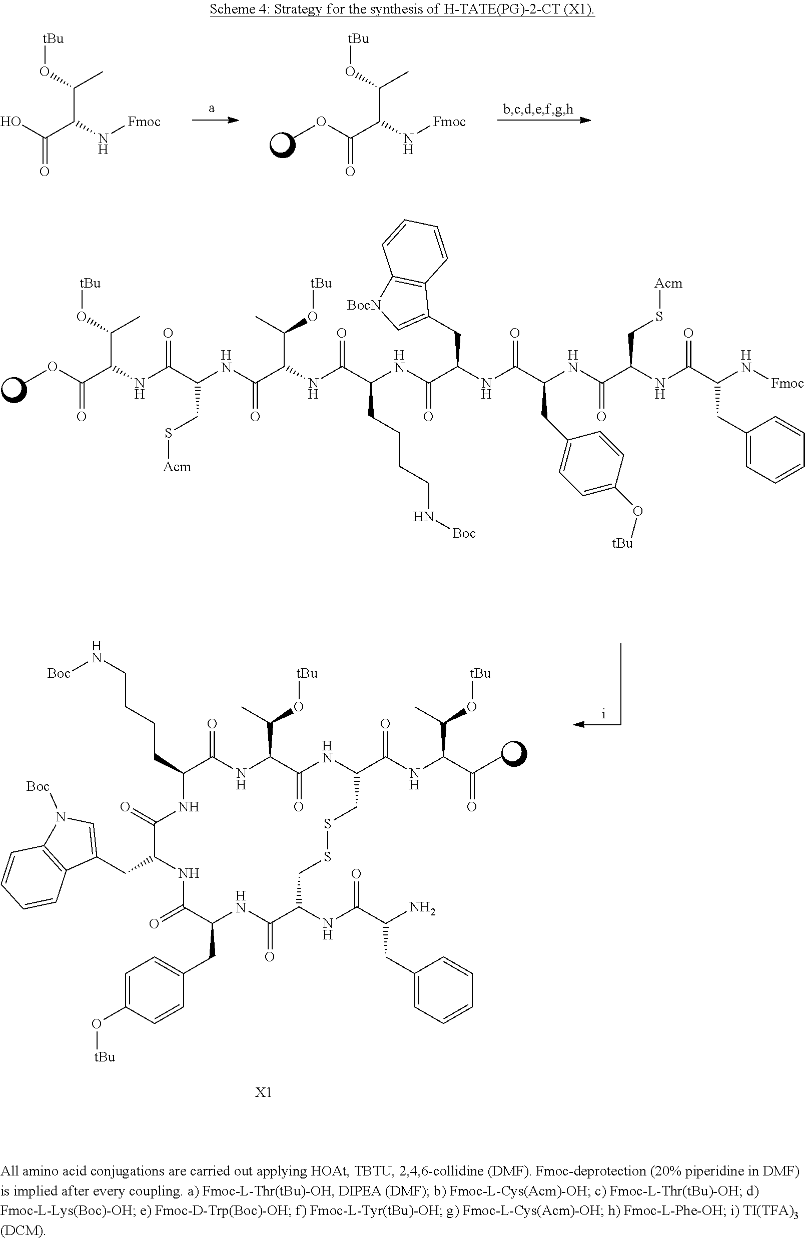

### 3.3.2 JR11 (X25)

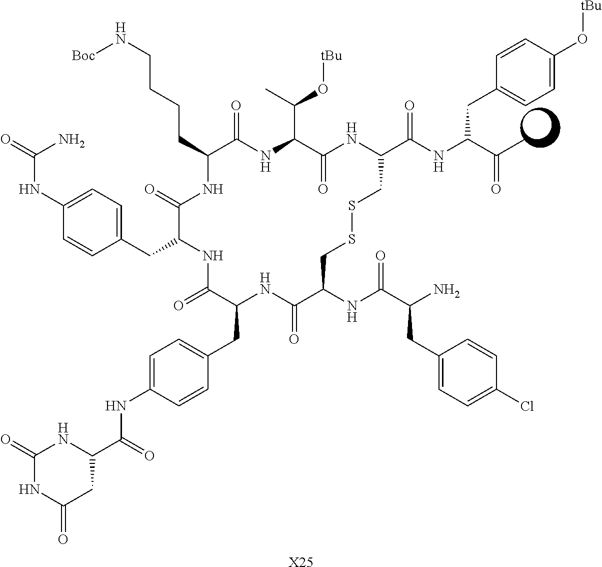

The on-resin synthesis of fully protected JR11 (X25) is carried out analogously to the synthesis described for X1 applying general procedures GP2 and GP4. In contrast to the synthesis described for TATE based peptides, the here applied resin is the Rink-amide resin. A specific loading protocol is not needed for this type of resin, therefore the first amino acid (Fmoc-D-Tyr(tBu)-OH, 1.5 eq.) is directly conjugated applying HOAt (1.5 eq.), TBTU (1.5 eq.) and DIPEA (4.0 eq.). Followed by Fmoc-deprotection and subsequent coupling of Fmoc-L-Cys(Acm)-OH, Fmoc-L-Thr(tBu)-OH, Fmoc-L-Lys(Boc)-OH, Fmoc-D-Aph(Cbm)-OH, Fmoc-L-Aph(Hor)-OH, Fmoc-o-Cys(Acm)-OH and Fmoc-L-Phe(4-Cl)—OH. For the conjugation of all amino acids the following protocol is applied: Fmoc-Xaa-OH (1.5 eq.), HOAt (1.5 eq.), TBTU (1.5 eq.) and DIPEA (4.0 eq.). The Fmoc protecting group of the last amino acid remains until the disulfide bridge is formed. The formation of the disulfide bridge is carried analogously to the synthesis described for X1. The completeness of the cyclization is verified by RP-HPLC and ESI-MS (test cleavage: TFA/H₂O/TIPS 90/2.5/7.5) confirming the synthesis of Fmoc-L-Phe(4-Cl)-cyclo[D-Cys-L-Aph(Hor)-D-Aph (Cbm)-L-Lys(Boc)-L-Thr(tBu)-L-Cys]-D-Tyr(tBu)-rink-amide. Finally, the peptide is Fmoc-deprotected leading to H-JR11(PG)-2-CT (X25).

RP-HPLC (10-90% in 15 min): t_{R}=9.90 min.

ESI-MS: m_{exact} (C₇₇H₉₀ClN₁₅O₁₆S₂): 1522.5; m_{found}: m/z=763.7 [M+2H]²⁺, 1524.2 [M+H]⁺.

### 3.4 Synthesis of Ligands

### 3.4.1 Synthesis of Precursors

### Synthesis of H-PEG₁-TATE(PG)-2-CT (X4)

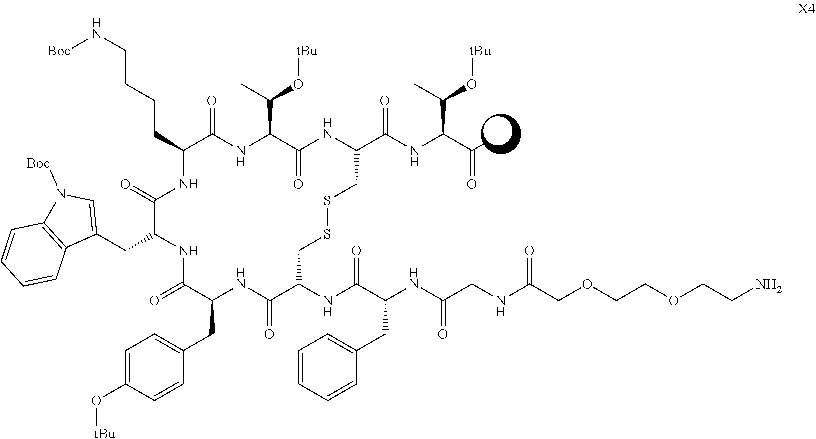

The synthesis of H-PEG₁-TATE(PG)-2-CT (X4) is carried out applying GP2 and GP4. Briefly, resin bound X1 (1.0 eq.) is conjugated with Fmoc-O20c-OH (Fmoc-PEG₁-OH, 1.50 eq.) applying HOAt (1.5 eq.) TBTU (1.5 eq.) and 2,4,6-collidine (5.5 eq.). Final Fmoc-deprotection results in compound X4.

### Synthesis of H-[Gly]₁₋₃-TATE(PG)-2-CT (X12, X5, X13)

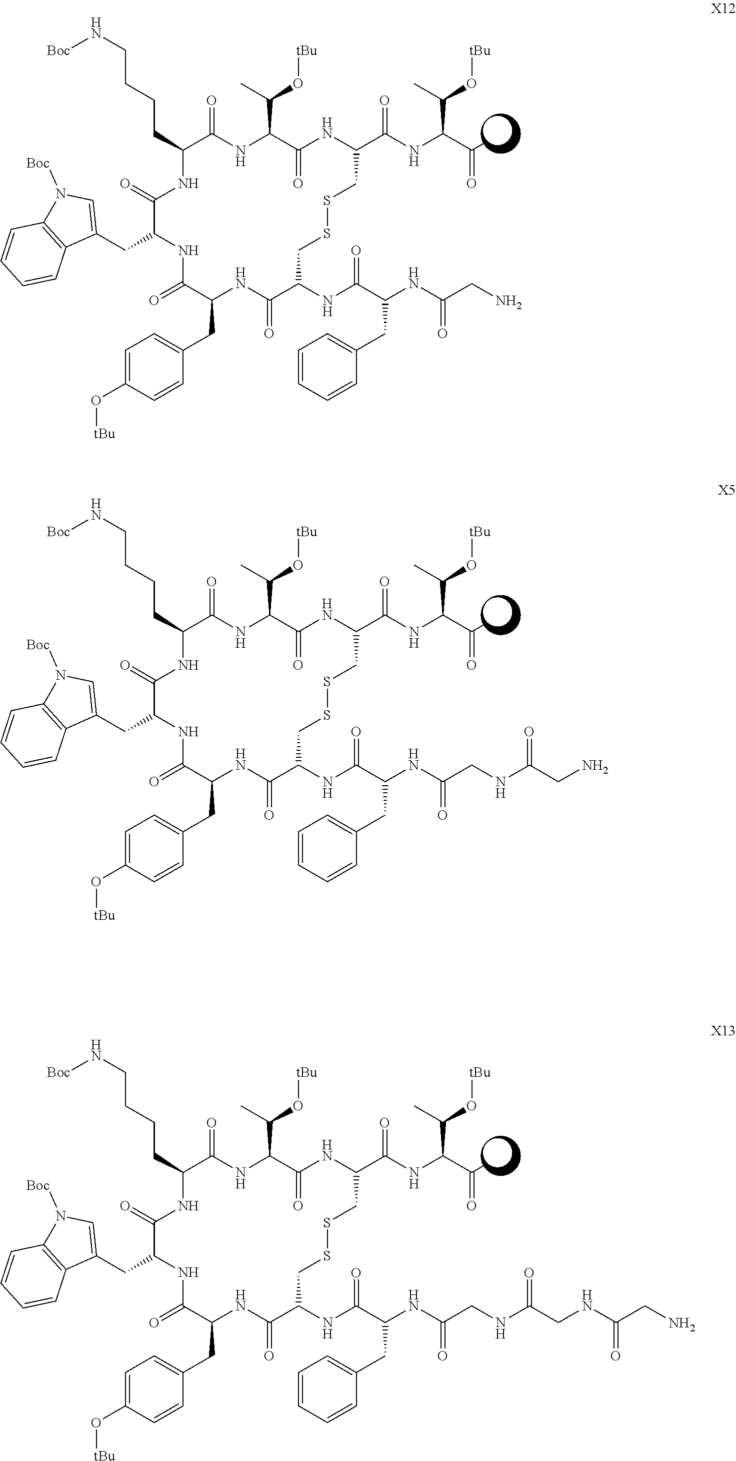

The synthesis of H-Gly-TATE(PG)-2-CT (X12), H-Gly-Gly-TATE(PG)-2-CT (X5) and H-Gly-Gly-Gly-TATE(PG)-2-CT (X13), are carried out analogously, applying GP2 and GP4. Briefly, resin bound X1 (1.0 eq.) is conjugated once, twice or thrice with Fmoc-Gly-OH (1.5 eq.) applying HOAt (1.5 eq.) TBTU (1.5 eq.) and 2,4,6-collidine (5.5 eq.). Final Fmoc-deprotection results in compounds X12, X5, and X13.

### Synthesis of H-L/D-Asn(Trt)-L/D-Asn(Trt)-TATE(PG)-2-CT (X6 and X16)

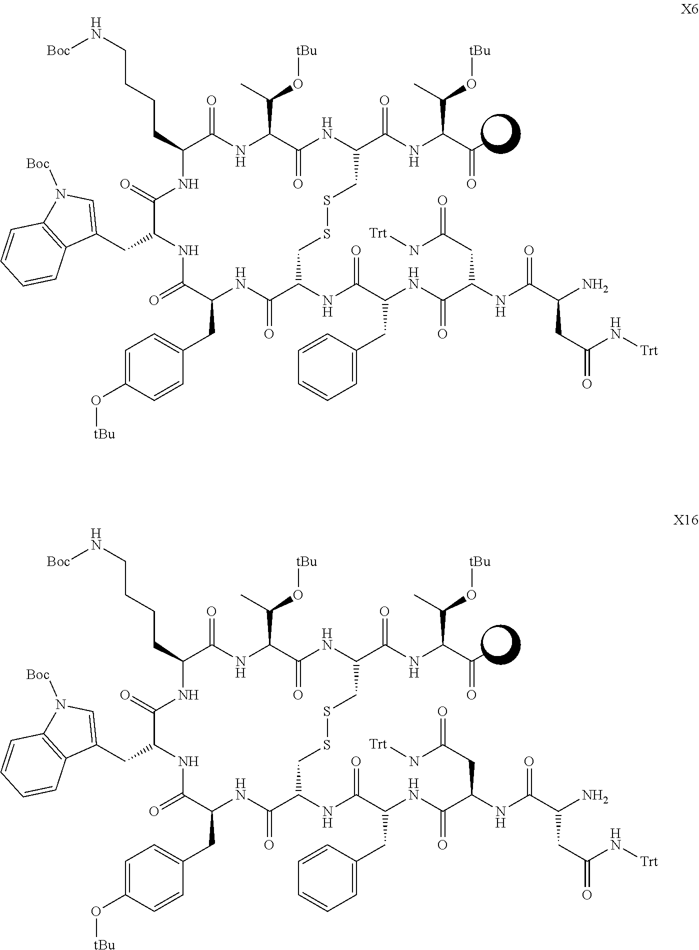

The synthesis of H-L-Asn(Trt)-L-Asn(Trt)-TATE(PG)-2-CT (X6) and H-D-Asn(Trt)-D-Asn(Trt)-TATE(PG)-2-CT (X16) are carried out, applying GP2 and GP4. Briefly, resin bound X1 (1.0 eq.) is conjugated twice with Fmoc-L-Asn(Trt)-OH (1.5 eq.) or Fmoc-D-Asn(Trt)-OH (1.5 eq.) applying HOAt (1.5 eq.) TBTU (1.5 eq.) and 2,4,6-collidine (5.5 eq.). Final Fmoc-deprotection results in compounds X6 and X16.

### Synthesis of H-Gly-D-Asn(Trt)-D-Asn(Trt)-TATE(PG)-2-CT (X17)

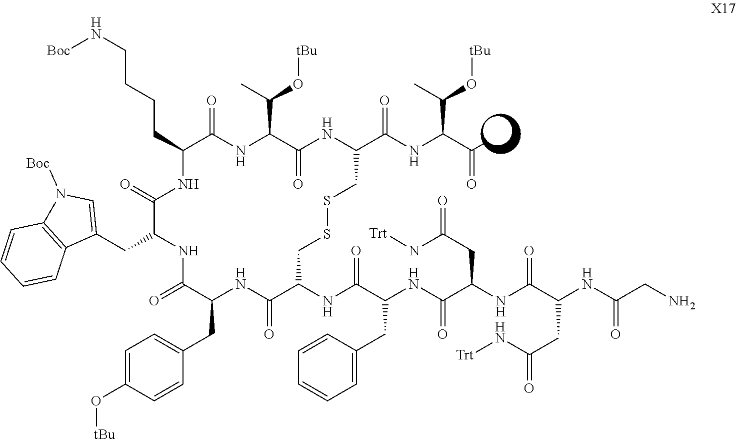

N The synthesis of H-Gly-D-Asn(Trt)-D-Asn(Trt)-TATE(PG)-2-CT (X17) is carried out, applying GP2 and GP4. Briefly, resin bound X1 (1.0 eq.) is conjugated twice with Fmoc-D-Asn(Trt)-OH (1.5 eq.) and once with Fmoc-Gly-OH, applying HOAt (1.5 eq.) TBTU (1.5 eq.) and 2,4,6-collidine (5.5 eq.). Final Fmoc-deprotection results in compound X17.

### H-D-Asp(tBu)-D-Asp(tBu)-PEG₁-TATE(PG)-2-CT (X7)

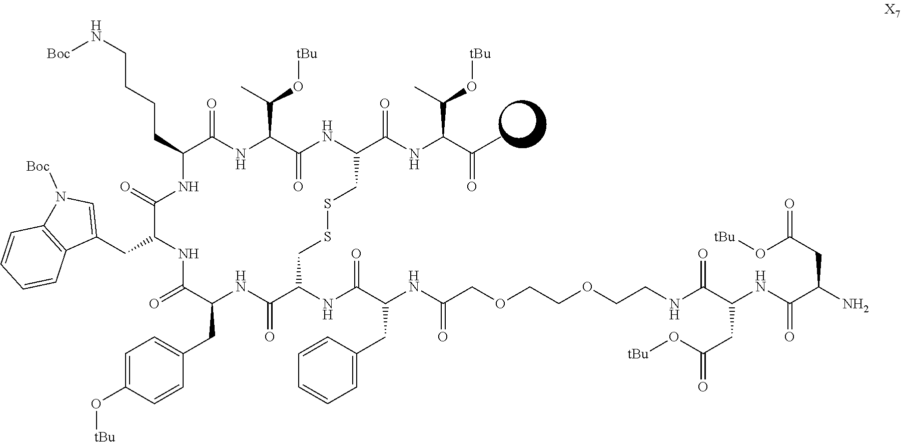

The synthesis of H-D-Asp(tBu)-D-Asp(tBu)-PEG₁-TATE(PG)-2-CT (X7), is carried out applying GP2 and GP4. Briefly, resin bound X4 (1.0 eq.) is conjugated twice with Fmoc-D-Asp(tBu)-OH (1.5 eq.) applying HOAt (1.5 eq.) TBTU (1.5 eq.) and 2,4,6-collidine (5.5 eq.). Final Fmoc-deprotection results in compound X7.

### H-D-Asp(tBu)-D-Asp(tBu)-Gly-TATE(PG)-2-CT (X14)

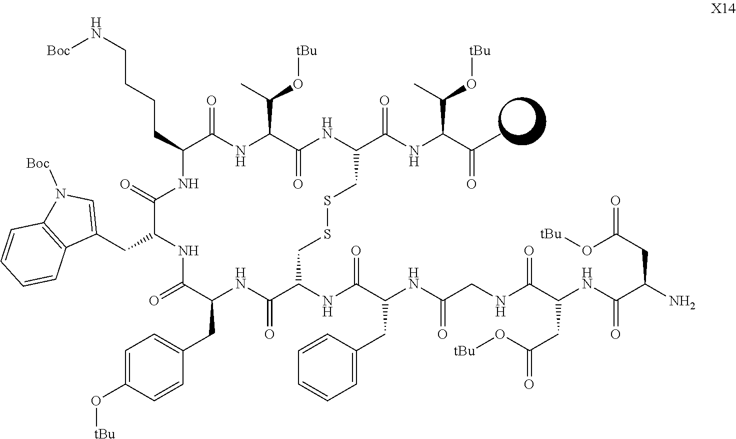

Starting from resin bound X12, the synthesis of H-D-Asp(tBu)-D-Asp(tBu)-Gly-TATE(PG)-2-CT (X14) is carried out analogously to the synthesis of X7. All synthesis steps are transferable.

### H-D-Asp(tBu)-D-Asp(tBu)-Gly-Gly-TATE(PG)-2-CT (X8)

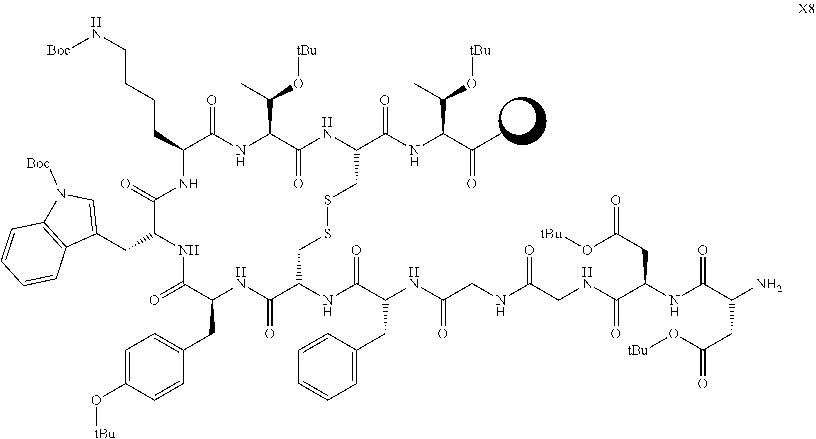

Starting from resin bound X5, the synthesis of H-D-Asp(tBu)-D-Asp(tBu)-Gly-Gly-TATE(PG)-2-CT (X8) is carried out analogously to the synthesis of X7. All synthesis steps are transferable.

### H-D-Asp(tBu)-D-Asp(tBu)-Gly-Gly-Gly-TATE(PG)-2-CT (X15)

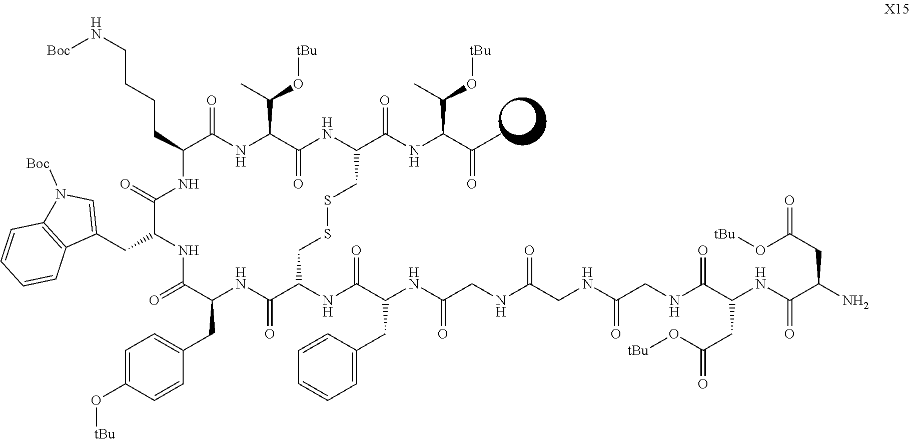

Starting from resin bound X13, the synthesis of H-D-Asp(tBu)-D-Asp(tBu)-Gly-Gly-Gly-TATE(PG)₂-CT (X15) is carried out analogously to the synthesis of X7. All synthesis steps are transferable.

### H-D-Asp(tBu)-D-Asp(tBu)-L-Asn(Trt)-L-Asn(Trt)-TATE(PG)-2-CT (X9)

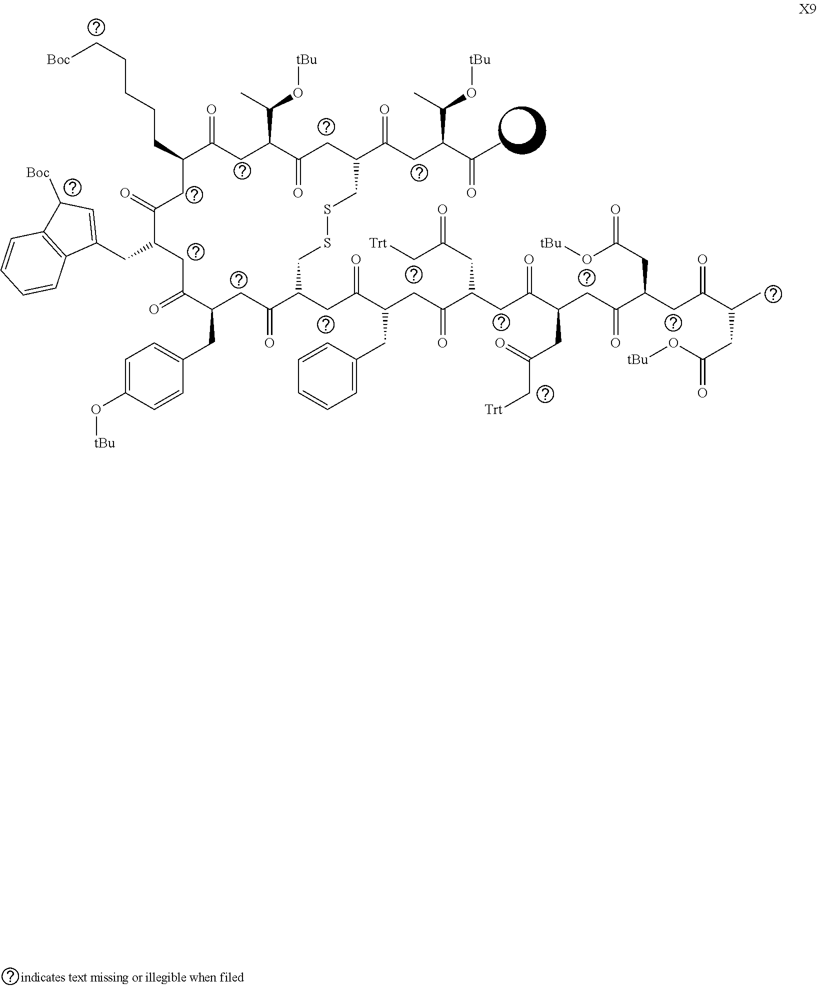

Starting from resin bound X6, the synthesis of H-D-Asp(tBu)-D-Asp(tBu)-L-Asn(Trt)-L-Asn(Trt)-TATE(PG)-2-CT (X9) is carried out analogously to the synthesis of X7. All synthesis steps are transferable.

H-D-Asp(tBu)-D-Asp(tBu)-D-Asn(Trt)-D-Asn(Trt)-TATE(PG)-2-CT (X18)

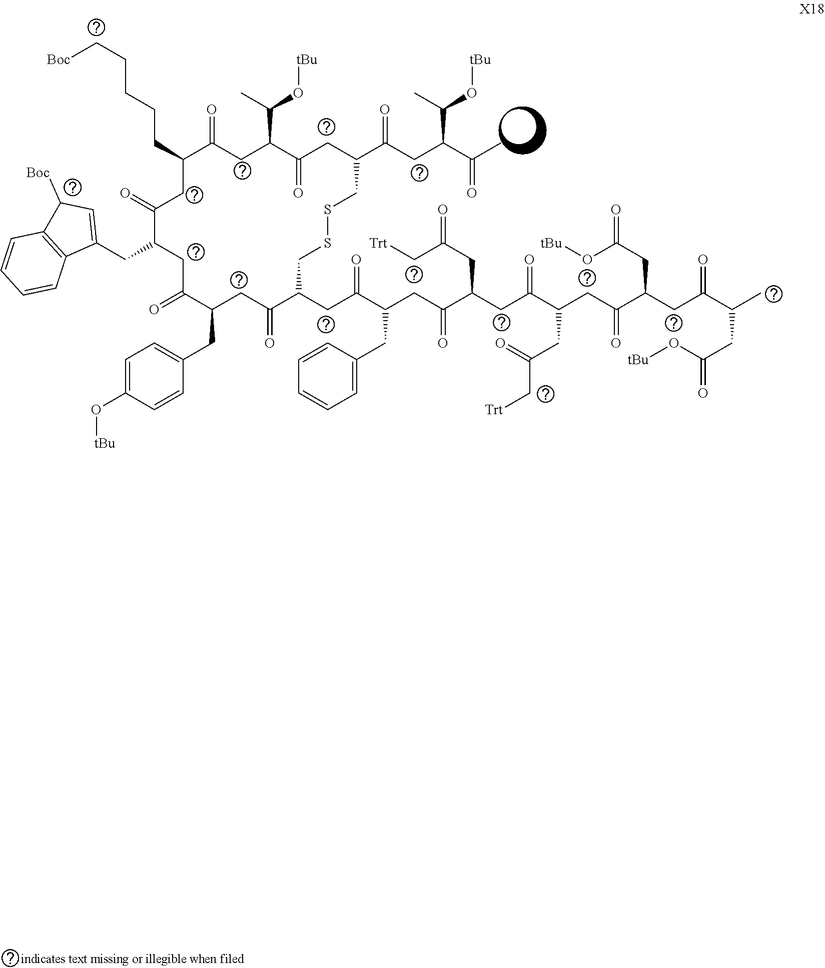

Starting from resin bound X16, the synthesis of H-D-Asp(tBu)-D-Asp(tBu)-D-Asn(Trt)-D-Asn(Trt)-TATE(PG)-2-CT (X18) is carried out analogously to the synthesis of X7. All synthesis steps are transferable.

### H-D-Asp(tBu)-D-Asp(tBu)-Gly-D-Asn(Trt)-D-Asn(Trt)-TATE(PG)-2-CT (X19)

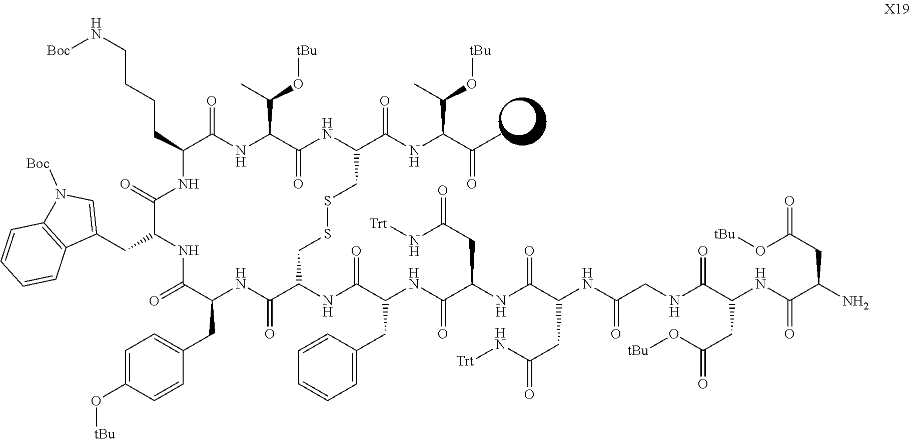

^{H} Starting from resin bound X17, the synthesis of H-D-Asp(tBu)-D-Asp(tBu)-D-Asn(Trt)-D-Asn(Trt)-TATE(PG)-2-CT (X19) is carried out analogously to the synthesis of X7. All synthesis steps are transferable.

### Synthesis of H-D-Orn(Boc)-D-Orn(Boc)-Gly-Gly-TATE(PG)-2-CT (X10)

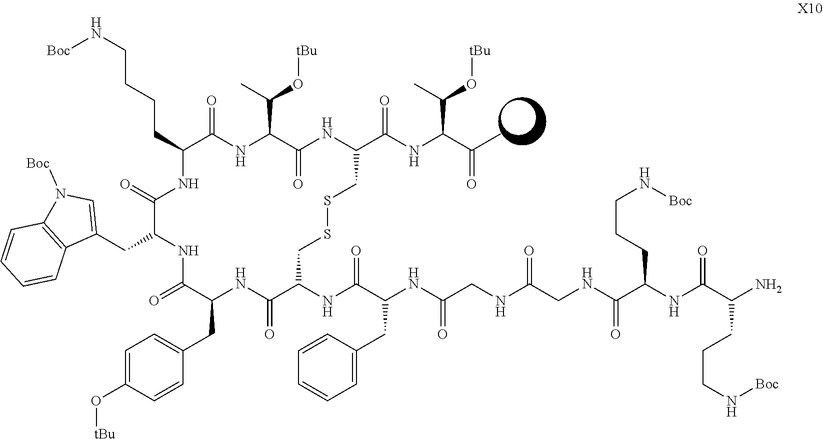

Starting from resin bound X5, the synthesis of H-D-Orn(Boc)-D-Orn(Boc)-Gly-Gly-TATE(PG)-2-CT (X10) is carried out analogously to the synthesis of X7. In contrast to X7, instead of Fmoc-D-Asp(tBu)-OH, Fmoc-D-Orn(Boc)-OH is used as amino acid. All other steps are transferable.

### Synthesis of H-D-Orn(Boc)-D-Orn(Boc)-Gly-Gly-Gly-TATE(PG)-2-CT (X23)

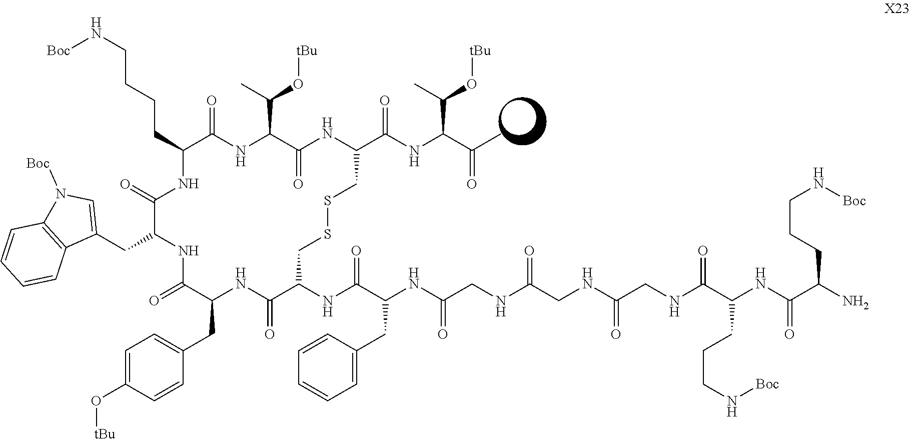

Starting from resin bound X13, the synthesis of H-D-Orn(Boc)-D-Orn(Boc)-Gly-Gly-Gly-TATE(PG)-2-CT (X23) is carried out analogously to the synthesis of X10. All Synthesis steps are transferable.

### Synthesis of H-D-Asn(Trt)-D-Asn(Trt)-Gly-Gly-TATE(PG)-2-CT (X11)

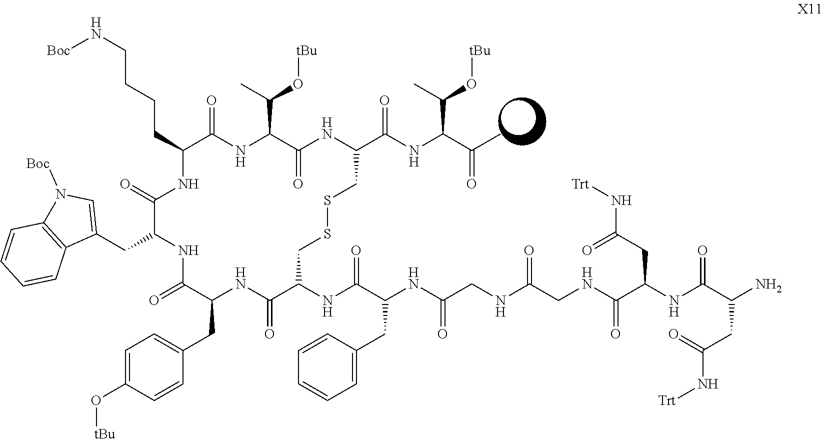

Starting from resin bound X5, the synthesis of H-D-Asn(Trt)-D-Asn(Trt)-Gly-Gly-TATE(PG)-2-CT (X11) is carried out analogously to the synthesis of X7. In contrast to X7, instead of Fmoc-D-Asp(tBu)-OH, Fmoc-D-Asn(Trt)-OH is used as amino acid. All other steps are transferable.

### Synthesis of H-D-Asn(Trt)-D-Asn(Trt)-Gly-Gly-Gly-TATE(PG)-2-CT (X24)

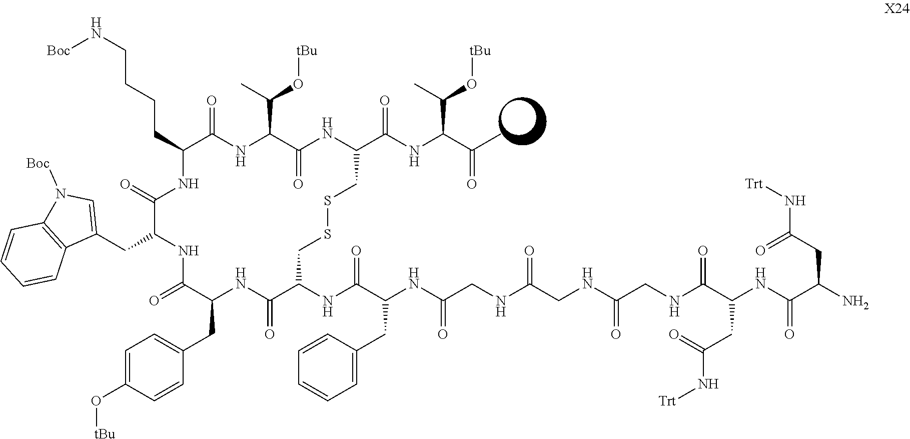

Starting from resin bound X13, the synthesis of H-D-Asn(Trt)-D-Asn(Trt)-Gly-Gly-Gly-TATE(PG)-2-CT (X24) is carried out analogously to the synthesis of X11. All synthesis steps are transferable.

### Synthesis of H-D-Lys(Boc)-D-Asp(tBu)-D-Asp(tBu)-Gly-Gly-TATE(PG)-2-CT (X20), H-D-Cit-D-Asp(tBu)-D-Asp(tBu)-Gly-Gly-TATE(PG)-2-CT (X21) and H-D-Glu(tBu)-D-Asp(tBu)-D-Asp(tBu)-Gly-Gly-TATE(PG)-2-CT (X22)

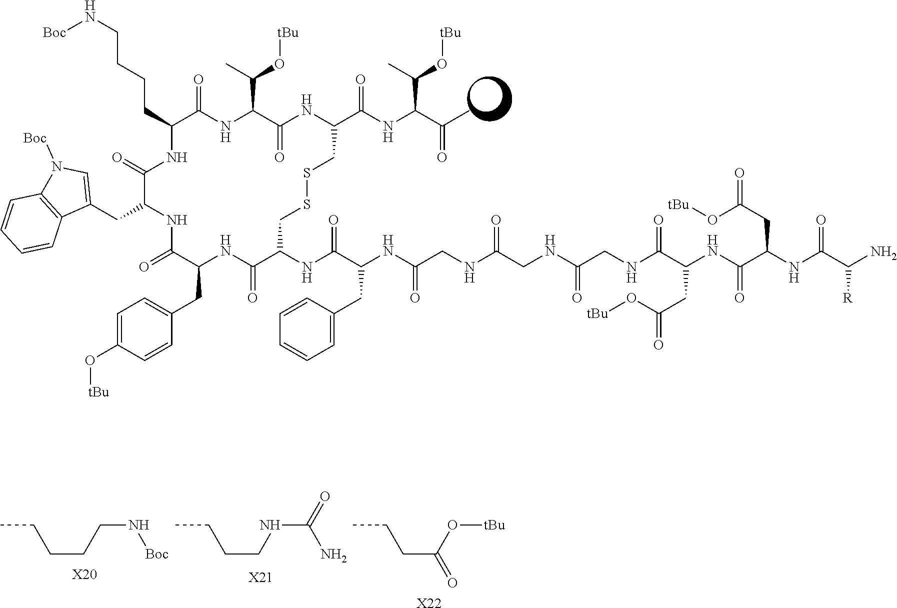

The synthesis of X20, X21 and X22 are carried out applying GP2 and GP4. Briefly, resin bound X15 (1.0 eq.) is conjugated with either Fmoc-D-Lys(Boc)-OH (1.5 eq.) or Fmoc-D-Cit-OH (1.5 eq.) or Fmoc-D-Glu(tBu)-OH (1.5 eq.) applying HOAt (1.5 eq.) TBTU (1.5 eq.) and 2,4,6-collidine (5.5 eq.). Final Fmoc-deprotection results in compounds X20, X21 and X22.

### H-D-Asp(tBu)-D-Asp(tBu)-Gly-Gly-Gly-JR11(PG)-rink-amide (X26)

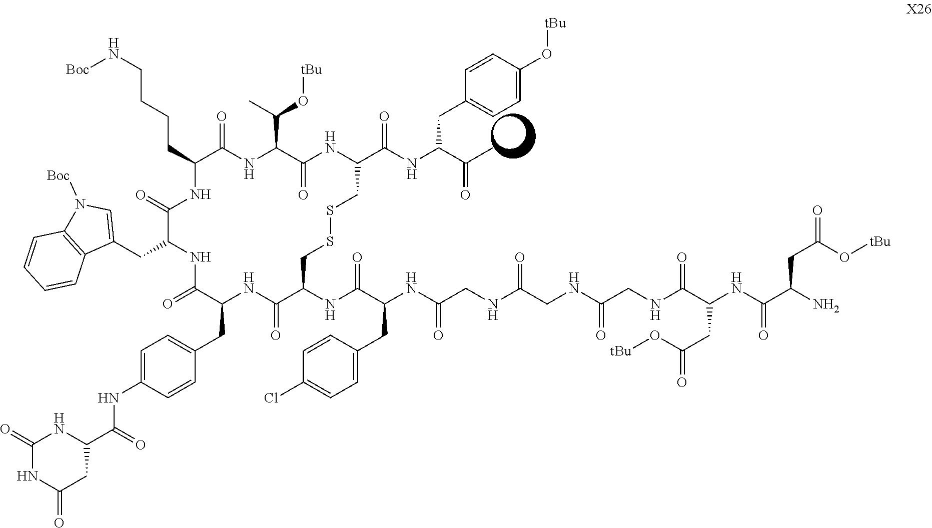

The synthesis of H-D-Asp(tBu)-D-Asp(tBu)-Gly-Gly-Gly-JR11(PG)-rink-amide (X26), is carried out applying GP2 and GP4. Briefly, resin bound X25 (1.0 eq.) is conjugated three times with Fmoc-Gly-OH and then twice with Fmoc-D-Asp(tBu)-OH (1.5 eq.) applying HOAt (1.5 eq.) TBTU (1.5 eq.) and DIPEA (4.0 eq.). Final Fmoc-deprotection results in compound X26.

### 3.4.2 Ligand Synthesis: Simple SiFA-Moiety

### Synthesis of 1 (Reference Ligand)

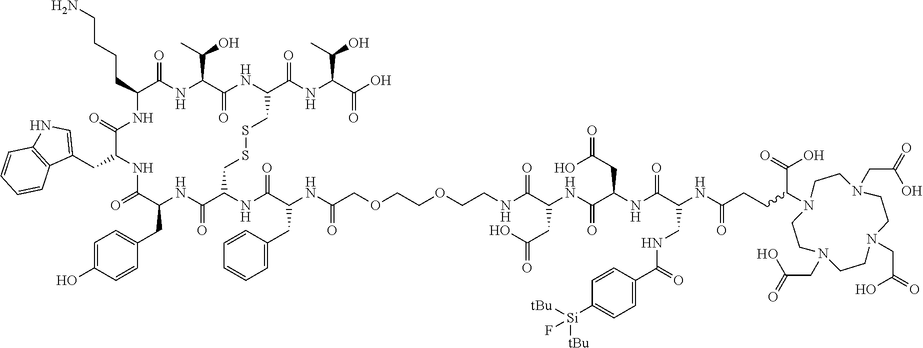

The synthesis of the ligand 1 is carried out applying GP2, GP3, GP4, GP5 and GP7. Briefly, resin bound precursor X7 (1.0 eq.) is conjugated OH (1.5 eq.) applying HOBt (1.5 eq.) TBTU (1.5 eq.) and 2,4,6-collidine (5.5 eq.). After Dde-deprotection, SiFA-BA (1.5 eq.) is conjugated applying HOBt (1.5 eq.), TBTU (1.5 eq.) and DIPEA (4.5 eq.). After Fmoc-deprotection, the N-terminus (1.0 eq.) is conjugated with the chelator rac-DOTAGA-anhydride (2.5 eq.), applying DIPEA (10.0 eq.) in DMF (8 mL/g resin) for 24 h at room temperature. The resin bound peptide is deprotected and cleaved from the resin by treatment with TFA/TIPS/H₂O, as described in GP7. The crude product is purified applying semi-preparative RP-HPLC and the solvent is removed under reduced pressure. The precipitate is dissolved in tBuOH/H₂O, frozen at −80° C. and final lyophilisation yields peptide 1 as colorless solid.

RP-HPLC semi-preparative (38-55% in 20 min): t_{R}=11.8 min.

RP-HPLC (10-90% in 15 min): t_{R}=10.9 min.

ESI-MS: m_{exact} (C₁₀₀H₁₄₂FN₁₉O₃₂S₂Si): 2231.9; m_{found}: m/z=745.3 [M+3H]³⁺, 1117.2 [M+2H]²⁺, 1489.4 [2M+3H]³⁺.

### Synthesis of 3 (Reference Ligand)

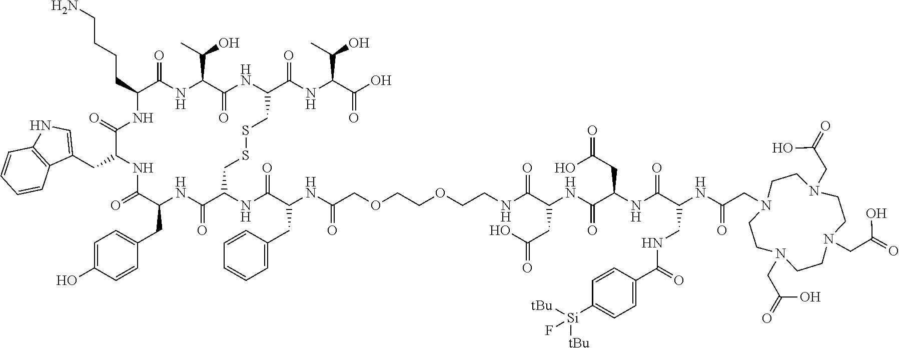

Starting from precursor X7, the synthesis of ligand 3 is carried out analogously to the synthesis described for 1. In contrast to the synthesis of 1, DOTA-(tBu)₃ (1.2 eq.) is used as chelator, applying HOBt (1.2 eq.), TBTU (1.2 eq.) and DIPEA (3.6 eq.). All other synthesis steps are transferable. After deprotection and cleavage from the resin, the crude product is purified applying semi-preparative RP-HPLC, yielding peptide 3 as colorless solid.

RP-HPLC semi-preparative (38-55% in 20 min): t_{R}=10.7 min.

RP-HPLC (10-90% in 15 min): t_{R}=8.84 min.

ESI-MS: m_{exact} (C₉₇H₁₃₈FN₁₉O₃₀S₂Si): 2159.9; m_{found}: m/z=721.1 [M+3H]³⁺, 1081.1 [M+2H]²⁺, 1441.1 [2M+3H]³⁺.

### Synthesis of 6 (Reference Ligand)

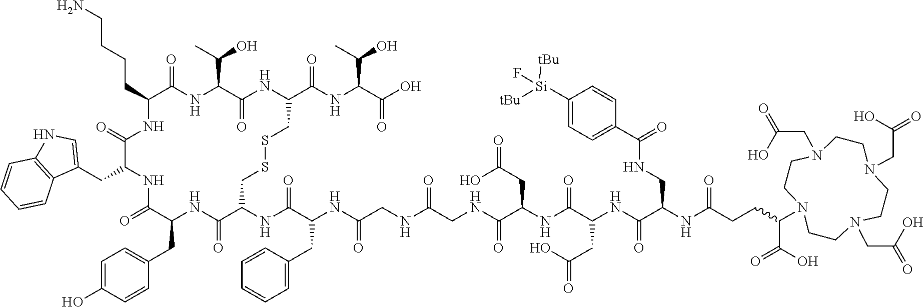

Starting from precursor X8, the synthesis of ligand 6 is carried out analogously to the synthesis described for 1. All synthesis steps are transferable. After deprotection and cleavage from the resin, the crude product is purified applying semi-preparative RP-HPLC, yielding peptide 6 as colorless solid.

RP-HPLC semi-preparative (38-55% in 20 min): t_{R}=10.5 min.

RP-HPLC (10-90% in 15 min): t_{R}=8.67 min.

ESI-MS: m_{exact} (C₉₈H₁₃₇FN₂₀O₃₁S₂Si): 2200.9; m_{found}: m/z=735.2 [M+3H]³⁺, 1101.9 [M+2H]²⁺, 1468.4 [2M+3H]³⁺.

### Synthesis of 8 (Reference Ligand)

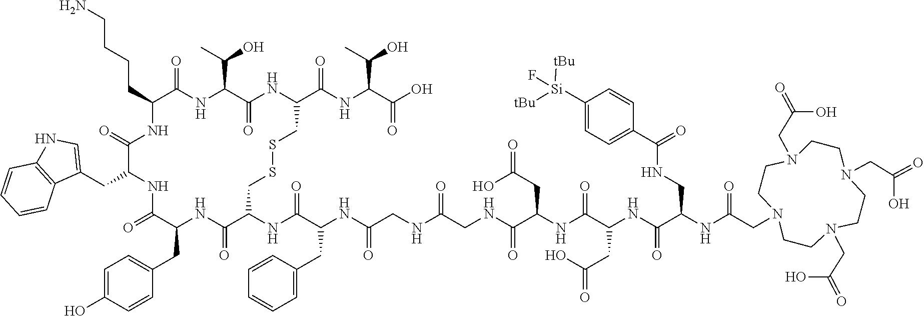

Starting from precursor X8, the synthesis of ligand 8 is carried out analogously to the synthesis described for 1. In contrast to the synthesis of 1, DOTA(tBu)₃ (1.2 eq.) is used as chelator, applying HOBt (1.2 eq.), TBTU (1.2 eq.) and DIPEA (3.6 eq.). All other synthesis steps are transferable. After deprotection and cleavage from the resin, the crude product is purified applying semi-preparative RP-HPLC, yielding peptide 8 as colorless solid.

RP-HPLC semi-preparative (38-55% in 20 min): t_{R}=10.7 min.

RP-HPLC (10-90% in 15 min): t_{R}=8.74 min.

ESI-MS: m_{exact} (C₉₅H₁₃₃FN₂₀O₂₉S₂Si): 2128.9; m_{found}: m/z=711.3 [M+3H]³⁺, 1065.7 [M+2H]²⁺.

### Synthesis of 2 (Reference Ligand)

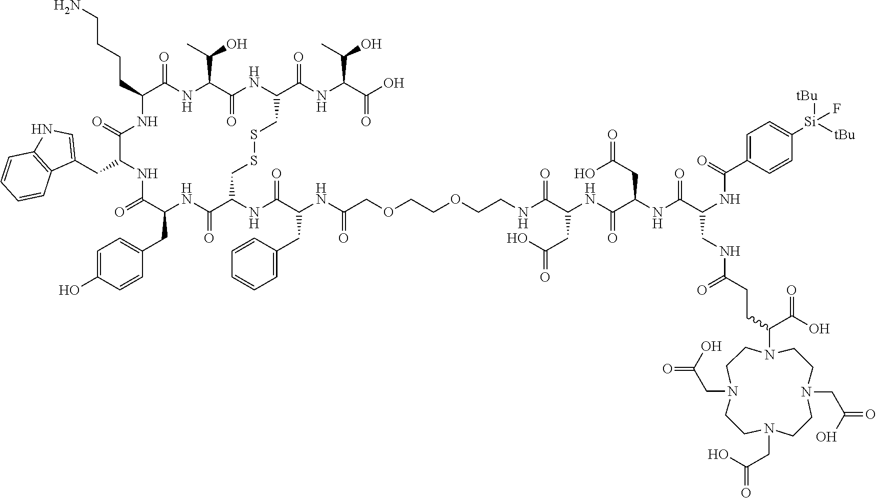

The synthesis of the ligand 2 is carried out applying GP2, GP3, GP4, GP5 and GP7. Briefly, resin bound precursor X7 (1.0 eq.) is conjugated with Fmoc-D-Dap(Dde)-OH (1.5 eq.) applying HOBt (1.5 eq.) TBTU (1.5 eq.) and 2,4,6-collidine (5.5 eq.). After Fmoc-deprotection, SiFA-BA (1.5 eq.) is conjugated applying HOBt (1.5 eq.), TBTU (1.5 eq.) and DIPEA (4.5 eq.). After Dde-deprotection, the N-terminus (1.0 eq.) is conjugated with the chelator rac-DOTAGA-anhydride (2.5 eq.), applying DIPEA (10.0 eq.) in DMF (8 mL/g resin) for 24 h at room temperature. The resin bound peptide is deprotected and cleaved from the resin by treatment with TFA/TIPS/H₂O, as described in GP7. The crude product is purified applying semi-preparative RP-HPLC and the solvent is removed under reduced pressure. The precipitate is dissolved in tBuOH/H₂O, frozen at −80° C. and final lyophilisation yields peptide 1 as colorless solid.

RP-HPLC semi-preparative (38-55% in 20 min): t_{R}=11.5 min.

RP-HPLC (10-90% in 15 min): t_{R}=10.7 min.

ESI-MS: m_{exact} (C₁₀₀H₁₄₂FN₁₉O₃₂S₂Si): 2231.9; m_{found}: m/z=745.3 [M+3H]³⁺, 1117.2 [M+2H]²⁺, 1489.4 [2M+3H]³⁺.

### Synthesis of 4 (Reference Ligand)

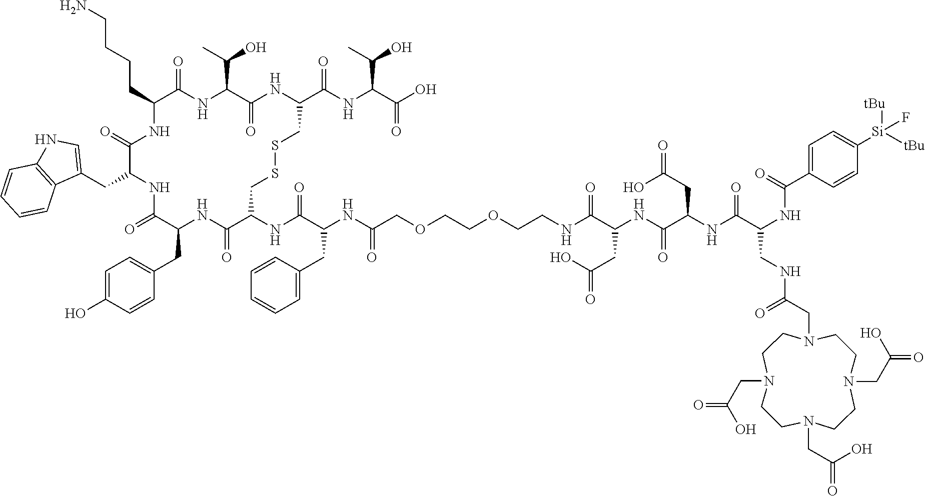

Starting from precursor X7, the synthesis of ligand 4 is carried out analogously to the synthesis described for 2. In contrast to the synthesis of 2, DOTA (tBu)₃ (1.2 eq) is used as chelator, applying HOBt (1.2 eq.), TBTU (1.2 eq.) and DIPEA (3.6 eq.). All other synthesis steps are transferable. After deprotection and cleavage from the resin, the crude product is purified applying semi-preparative RP-HPLC, yielding peptide 4 as colorless solid.

RP-HPLC semi-preparative (38-55% in 20 min): t_{R}=10.6 min.

RP-HPLC (10-90% in 15 min): t_{R}=8.83 min.

ESI-MS: m_{exact} (C₉₇H₁₃₈FN₁₉O₃₀S₂Si): 2159.9; m_{found}: m/z=721.1 [M+3H]³⁺, 1081.1 [M+2H]²⁺, 1441.1 [2M+3H]³⁺.

### Synthesis of 7 (Reference Ligand)

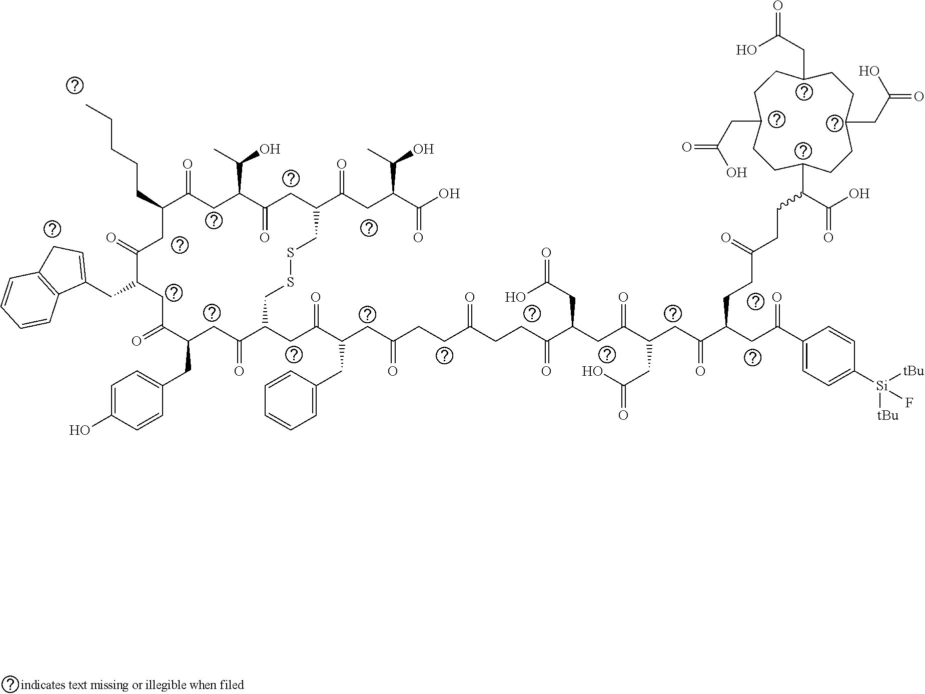

Starting from precursor X8, the synthesis of ligand 7 is carried out analogously to the synthesis described for 2. All synthesis steps are transferable. After deprotection and cleavage from the resin, the crude product is purified applying semi-preparative RP-HPLC, yielding peptide 7 as colorless solid.

RP-HPLC semi-preparative (38-55% in 20 min): t_{R}=10.5 min.

RP-HPLC (10-90% in 15 min): t_{R}=8.64 min.

ESI-MS: m_{exact} (C₉₈H₁₃₇FN₂₀O₃₁S₂Si): 2200.9; m_{found}: m/z=735.2 [M+3H]³⁺, 1101.9 [M+2H]²⁺, 1468.4 [2M+3H]³⁺.

### Synthesis of 9 (Reference Ligand)

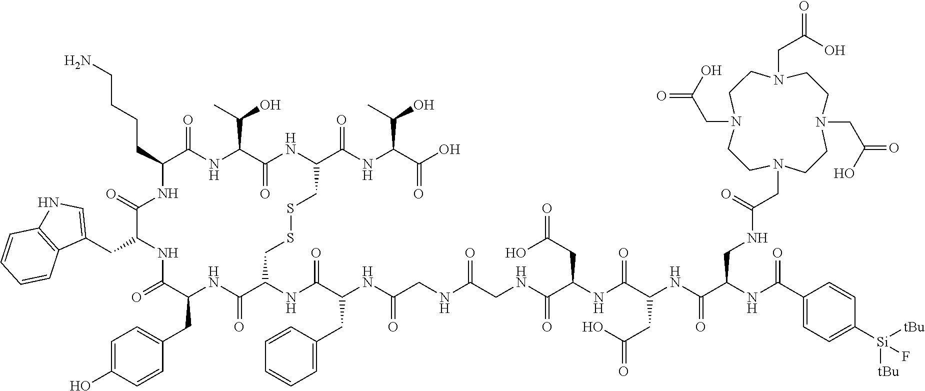

Starting from precursor X8, the synthesis of ligand 9 is carried out analogously to the synthesis described for 2. In contrast to the synthesis of 2, DOTA(tBu)₃ (1.2 eq.) is used as chelator, applying HOBt (1.2 eq.), TBTU (1.2 eq.) and DIPEA (3.6 eq.). All other synthesis steps are transferable. After deprotection and cleavage from the resin, the crude product is purified applying semi-preparative RP-HPLC, yielding peptide 9 as colorless solid.

RP-HPLC semi-preparative (38-55% in 20 min): t_{R}=11.0 min.

RP-HPLC (10-90% in 15 min): t_{R}=8.89 min.

ESI-MS: m_{exact} (C₉₅H₁₃₃FN₂₀O₂₉S₂Si): 2128.9; m_{found}: m/z=711.3 [M+3H]³⁺, 1065.7 [M+2H]²⁺, 1420.7 [2M+3H]³⁺.

### Synthesis of 18 (Reference Ligand)

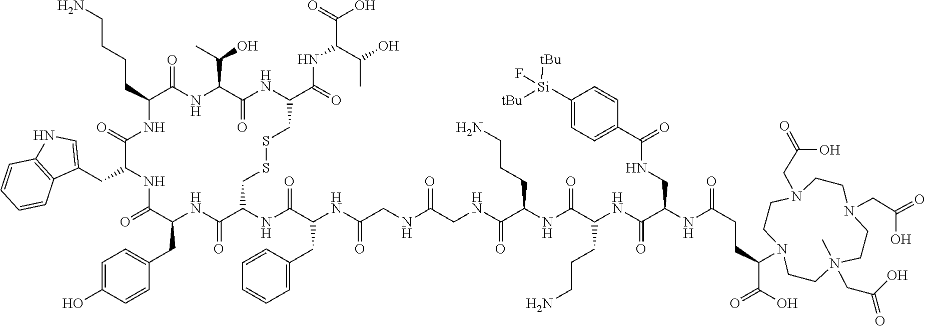

The synthesis of the ligand 18 is carried out applying GP2, GP3, GP4, GP5 and GP7. Briefly, resin bound precursor X10 (1.0 eq.) is conjugated with Fmoc-D-Dap(Dde)-OH (1.5 eq.) applying HOAt (1.5 eq.) TBTU (1.5 eq.) and 2,4,6-collidine (5.5 eq.). After Dde-deprotection, SiFA-BA (1.5 eq.) is conjugated applying HOAt (1.5 eq.), TBTU (1.5 eq.) and 2,4,6-collidine (5.5 eq.) and the N-terminus is Fmoc-deprotected. R-DOTAGA(tBu)₄ is conjugated applying HOAt (1.5 eq.), HATU (1.5 eq.) and DIPEA (4.5 eq.). The resin bound peptide is deprotected and cleaved from the resin by treatment with TFA/TIPS/H₂O, as described in GP7. The crude product is purified applying semi-preparative RP-HPLC and the solvents are removed under reduced pressure. The precipitate is dissolved in tBuOH/H₂O, frozen at −80° C. and final lyophilisation yields peptide 18 as colorless solid.

RP-HPLC semi-preparative (30-50% in 20 min): t_{R}=16.1 min.

RP-HPLC (10-60% in 15 min): t_{R}=11.2 min.

ESI-MS: m_{exact} (C₁₀₀H₁₄₇FN₂₂O₂₇S₂Si): 2199.0; m_{found}: m/z=550.7 [M+4H]⁴⁺, 733.8 [M+3H]³⁺, 1099.9 [M+2H]²⁺, 1466.1 [2M+3H]³⁺.

### 3.4.3 Ligand Synthesis: Positive Charge Through Additional Dap at SiFA

### Synthesis of 23

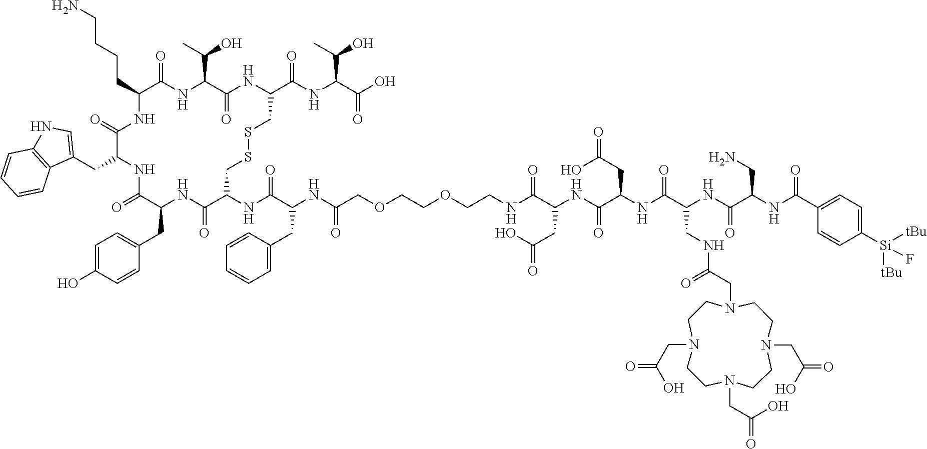

The synthesis of the ligand 23 is carried out applying GP2, GP3, GP4, GP5 and GP7. Briefly, resin bound precursor X7 (1.0 eq.) is conjugated with Fmoc-D-Dap(Dde)-OH (1.5 eq.) applying HOAt (1.5 eq.) TBTU (1.5 eq.) and 2,4,6-collidine (5.5 eq.). After Dde-deprotection, DOTA(tBu)₃ (1.5 eq.) is conjugated, applying HOAt (1.5 eq.), HATU (1.5 eq.) and DIPEA (4.5 eq.). The N-terminus is Fmoc-deprotected and conjugated with Fmoc-D-Dap(Boc)-OH (1.5 eq.) applying HOAt (1.5 eq.), TBTU (1.5 eq.) and 2,4,6-collidine (5.5 eq.), followed by Fmoc-deprotection. SiFA-BA (1.5 eq.) is conjugated applying HOAt (1.5 eq.), TBTU (1.5 eq.) and 2,4,6-collidine (5.5 eq.). The resin bound peptide is deprotected and cleaved from the resin by treatment with TFA/TIPS/H₂O, as described in GP7. The crude product is purified applying semi-preparative RP-HPLC and the solvents are removed under reduced pressure. The precipitate is dissolved in tBuOH/H₂O, frozen at −80° C. and final lyophilisation yields peptide 23 as colorless solid.

RP-HPLC semi-preparative (37-45% in 20 min): t_{R}=14.0 min.

RP-HPLC (10-60% in 15 min): t_{R}=12.2 min.

ESI-MS: m_{exact} (C₁₀₀H₁₄₄FN₂₁O₃₁S₂Si): 2246.0; m_{found}: m/z=749.6 [M+3H]³⁺, 1123.8 [M+2H]²⁺, 1499.0 [2M+3H]³⁺, 1685.7 [3M+4H]⁴⁺.

### Synthesis of 19

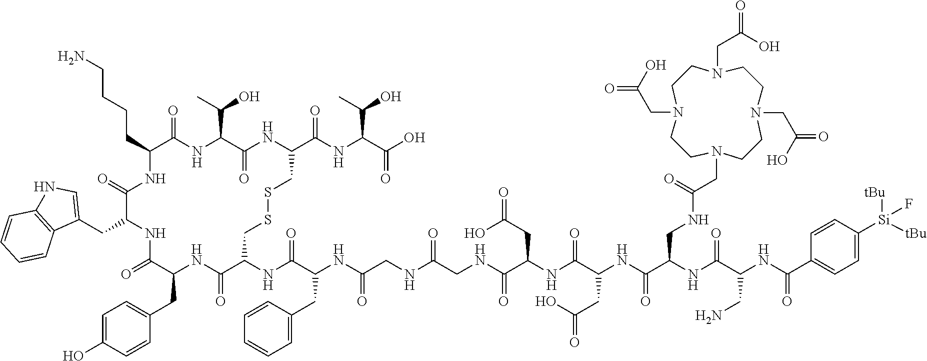

Starting from precursor X8, the synthesis of ligand 19 is carried out analogously to the synthesis described for 23. All synthesis steps are transferable. After deprotection and cleavage from the resin, the crude product is purified applying semi-preparative RP-HPLC, yielding peptide 19 as colorless solid.

RP-HPLC semi-preparative (33-45% in 20 min): t_{R}=17.6 min.

RP-HPLC (10-60% in 15 min): t_{R}=11.9 min.

ESI-MS: m_{exact} (C₉₈H₁₃₉FN₂₂O₃₀S₂Si): 2214.9; m_{found}: m/z=739.7 [M+3H]³⁺, 1108.5 [M+2H]²⁺, 1477.8 [2M+3H]³⁺, 1662.5 [3M+4H]⁴⁺.

### Synthesis of 39

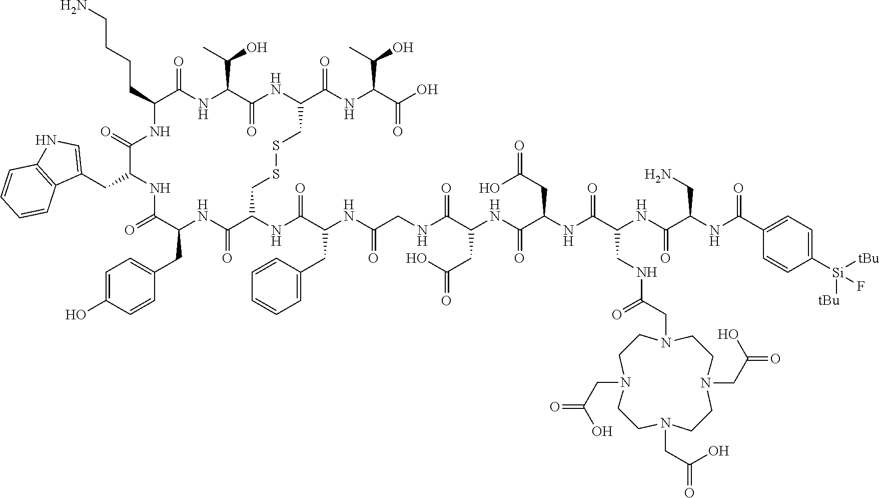

Starting from precursor X14, the synthesis of ligand 39 is carried out analogously to the synthesis described for 23. All synthesis steps are transferable. After deprotection and cleavage from the resin, the crude product is purified applying semi-preparative RP-HPLC, yielding peptide 39 as colorless solid.

RP-HPLC semi-preparative (34-44% in 20 min): t_{R}=15.5 min.

RP-HPLC (10-60% in 15 min): t_{R}=12.3 min.

ESI-MS: m_{exact} (C₉₆H₁₃₆FN₂₁O₂₉S₂Si): 2157.9; m_{found}: m/z=720.6 [M+3H]³⁺, 1080.5 [M+2H]²⁺, 1440.9 [2M+3H]³⁺, 1620.3 [3M+4H]⁴⁺.

### Synthesis of 40

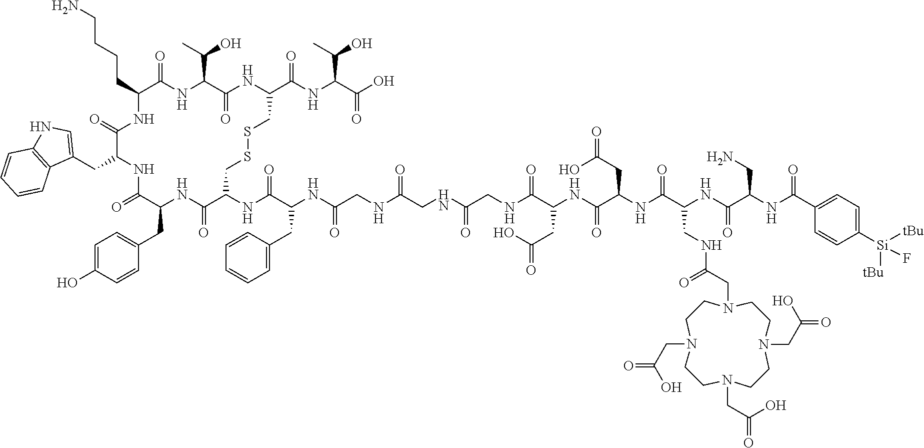

Starting from precursor X15, the synthesis of ligand 40 is carried out analogously to the synthesis described for 23. All synthesis steps are transferable. After deprotection and cleavage from the resin, the crude product is purified applying semi-preparative RP-HPLC, yielding peptide 40 as colorless solid.

RP-HPLC semi-preparative (37-44% in 20 min): t_{R}=14.8 mli.

RP-HPLC (10-60% in 15 min): t_{R}=12.0 min.

ESI-MS: m_{exact} (C₁₀₀H₁₄₂FN₂₃O₃₁S₂Si): 2271.9; m_{found}: m/z=758.7 [M+3H]³⁺, 1137.6 [M+2H]²⁺, 1517.2 [2M+3H]³⁺, 1706.5 [3M+4H]⁴⁺.

### Synthesis of 20

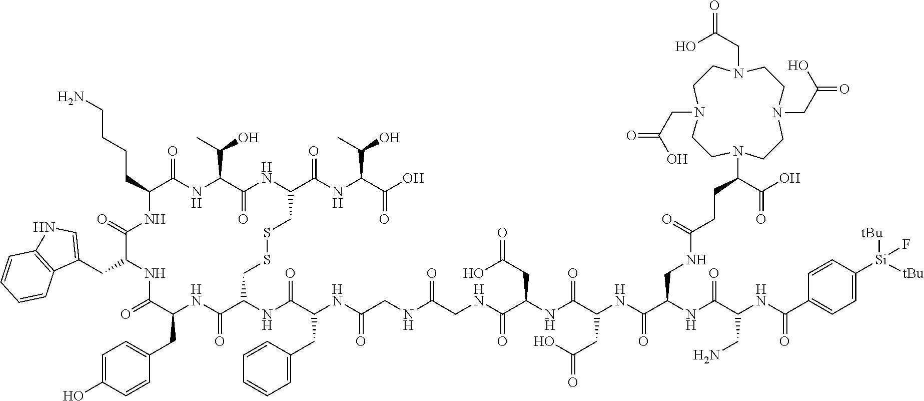

Starting from precursor X8, the synthesis of ligand 20 is carried out analogously to the synthesis described for 23. In contrast to the synthesis of 23, R-DOTAGA(tBu)₄ (1.5 eq.) is used as chelator, applying HOAt (1.5 eq.), HATU (1.5 eq.) and DIPEA (4.5 eq.). All other synthesis steps are transferable. After deprotection and cleavage from the resin, the crude product is purified applying semi-preparative RP-HPLC, yielding peptide 20 as colorless solid.

RP-HPLC semi-preparative (34-42% in 20 min): t_{R}=17.1 min.

RP-HPLC (10-60% in 15 min): t_{R}=11.7 min.

ESI-MS: m_{exact} (C₁₀₁H₁₄₃FN₂₂O₃₂S₂Si): 2286.9; m_{found}: m/z=572.9 [M+4H]⁴⁺, 763.7 [M+3H]³⁺, 1144.5 [M+2H]²⁺.

### Synthesis of 41

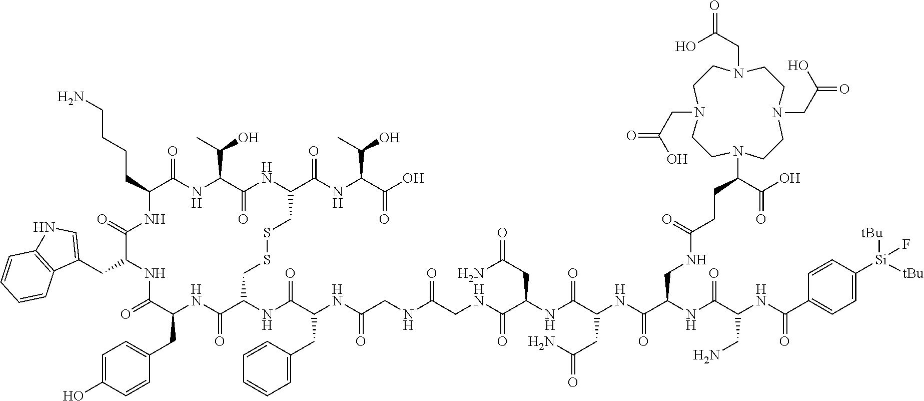

Starting from precursor X11, the synthesis of ligand 41 is carried out analogously to the synthesis described for 23. In contrast to the synthesis of 23, R-DOTAGA(tBu)₄ (1.5 eq.) is used as chelator, applying HOAt (1.5 eq.), HATU (1.5 eq.) and DIPEA (4.5 eq.). All other synthesis steps are transferable. After deprotection and cleavage from the resin, the crude product is purified applying semi-preparative RP-HPLC, yielding peptide 41 as colorless solid.

RP-HPLC semi-preparative (35-44% in 20 min): t_{R}=13.5 min.

RP-HPLC (10-60% in 15 min): t_{R}=11.7 min.

ESI-MS: m_{exact} (C₁₀₁H₁₄₅FN₂₄O₃₀S₂Si): 2285.0; m_{found}: m/z=763.1 [M+3H]³⁺, 1143.7 [M+2H]²⁺, 1525.4 [2M+3H]³⁺

### Synthesis of 42

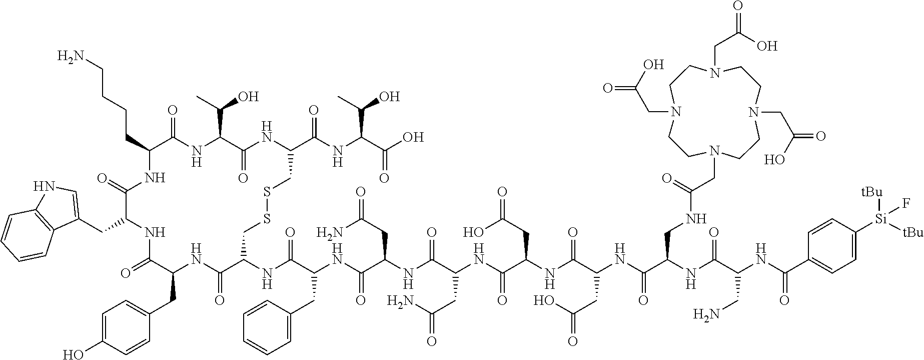

Starting from precursor X9, the synthesis of ligand 42 is carried out analogously to the synthesis described for 23. All synthesis steps are transferable. After deprotection and cleavage from the resin, the crude product is purified applying semi-preparative RP-HPLC, yielding peptide 42 as colorless solid.

RP-HPLC semi-preparative (35-45% in 20 min): t_{R}=14.6 min.

RP-HPLC (10-60% in 15 min): t_{R}=11.8 min.

ESI-MS: m_{exact} (C₁₀₂H₁₄₅FN₂₄O₃₂S₂Si): 2329.0; m_{found}: m/z=778.0 [M+3H]³⁺, 1166.9 [M+2H]²⁺, 1555.6 [2M+3H]³⁺.

### Synthesis of 43

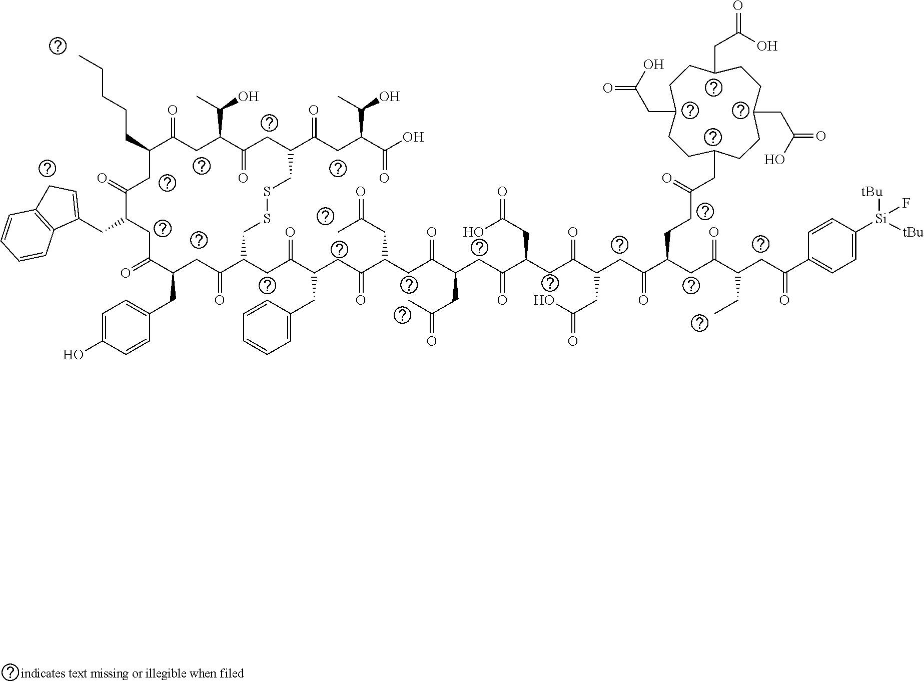

Starting from precursor X18, the synthesis of ligand 43 is carried out analogously to the synthesis described for 23. All synthesis steps are transferable. After deprotection and cleavage from the resin, the crude product is purified applying semi-preparative RP-HPLC, yielding peptide 43 as colorless solid.

RP-HPLC semi-preparative (35-45% in 20 min): t_{R}=15.4 min.

RP-HPLC (10-60% in 15 min): t_{R}=12.0 min.

ESI-MS: m_{exact} (C₁₀₂H₁₄₅FN₂₄O₃₂S₂Si): 2329.0; m_{found}: m/z=777.7 [M+3H]³⁺, 1165.8 [M+2H]²⁺, 1554.5 [2M+3H]³⁺.

### Synthesis of 36

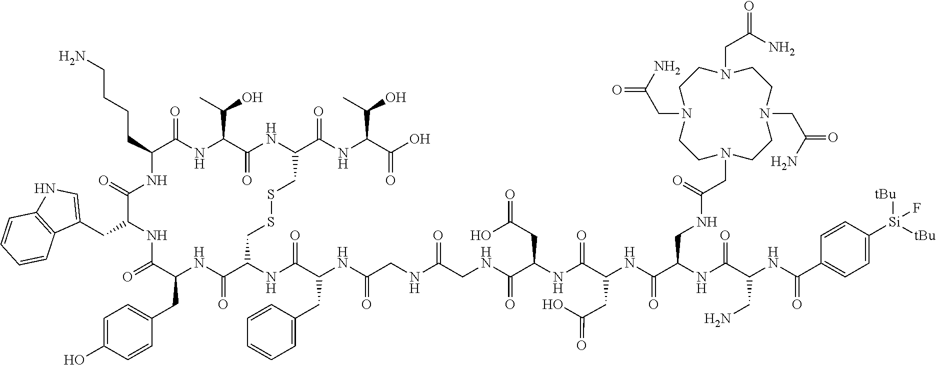

Starting from precursor X8, the synthesis of ligand 36 is carried out analogously to the synthesis described for 23. In contrast to the synthesis of 23, DO3AM-acetic acid (1.8 eq.) is used as chelator, applying HOAt (1.5 eq.), TBTU (1.5 eq.) and DIPEA (5.5 eq.) in DMF/NMP (1/1 v/v; 8 mL/g resin) for 3 h at room temperature. All other synthesis steps are transferable. After deprotection and cleavage from the resin, the crude product is purified applying semi-preparative RP-HPLC, yielding peptide 36 as colorless solid.

RP-HPLC semi-preparative (34-43% in 20 min): t_{R}=14.7 min.

RP-HPLC (10-60% in 15 min): t_{R}=11.3 min.

ESI-MS: m_{exact} (C₉₈H₁₄₂FN₂₅O₂₇S₂Si): 2212.0; m_{found}: m/z=728.4 [M+3H]³⁺, 1106.7 [M+2H]²⁺, 1475.9 [2M+3H]³⁺.

### Synthesis of 37

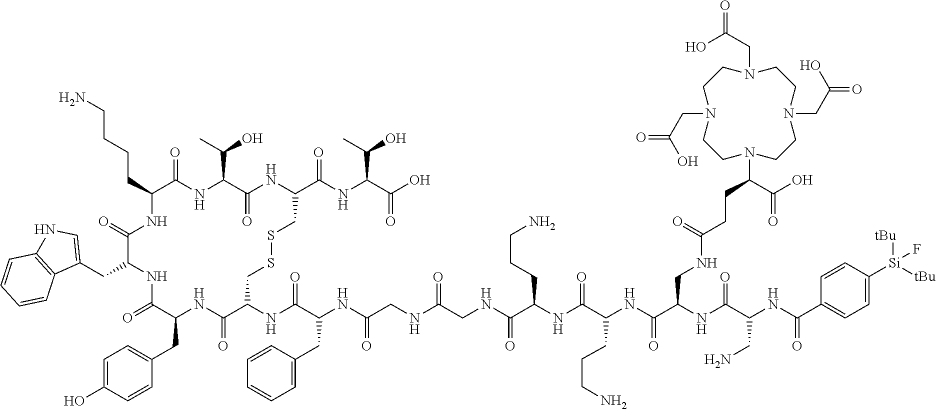

Starting from precursor X11, the synthesis of ligand 37 is carried out analogously to the synthesis described for 23. In contrast to the synthesis of 23, R-DOTAGA(tBu)₄ (1.5 eq.) is used as chelator, applying HOAt (1.5 eq.), HATU (1.5 eq.) and DIPEA (4.5 eq.). All other synthesis steps are transferable. After deprotection and cleavage from the resin, the crude product is purified applying semi-preparative RP-HPLC, yielding peptide 37 as colorless solid.

RP-HPLC semi-preparative (35-45% in 20 min): t_{R}=10.5 min.

RP-HPLC (10-60% in 15 min): t_{R}=10.8 min.

ESI-MS: m_{exact} (C₁₀₃H₁₅₃FN₂₄O₂₈S₂Si): 2285.1; m_{found}: m/z=573.1 [M+4H]⁴⁺, 763.9 [M+3H]³⁺.

3.4.4 Ligand Synthesis: positive Charge through SiFAlin-Building Block

### Synthesis of 24

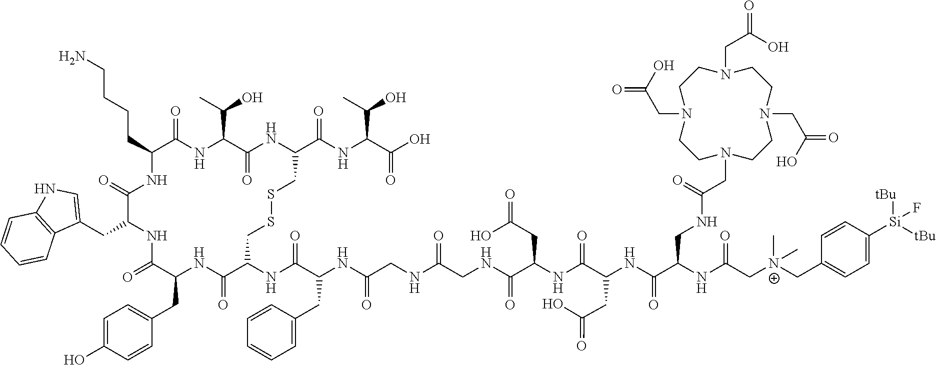

The synthesis of the ligand 24 is carried out applying GP2, GP3, GP4, GP5 and GP7. Briefly, resin bound precursor is conjugated with FMOc-D-Dap(Dde)-OH (1.5 eq.) applying HOAt (1.5 eq.) TBTU (1.5 eq.) and 2,4,6-collidine (5.5 eq.). After Dde-deprotection, DOTA(tBu)₃ (1.5 eq.) is conjugated, applying HOAt (1.5 eq.), HATU (1.5 eq.) and DIPEA (4.5 eq.). The N-terminus is Fmoc-deprotected and conjugated with Me₂-Gly-OH (3.0 eq.), applying HOAt (3.0 eq.), TBTU (3.0 eq.) and 2,4,6-collidine (9.0 eq.) in DMF (8 mL/g resin) for 4 h at room temperature. The SiFA-moiety is formed, by incubation with SiFA-Br (3.0 eq.) and 2,4,6-collidine (6.0 eq.) in DCM (8 mL/g resin) for 24 h at room temperature. Reaction control via test cleavage (GP8; mild test cleavage) confirms correct but incomplete product formation. Nevertheless, the resin bound peptide is deprotected and cleaved from the resin by treatment with TFA/TIPS/H₂O, as described in GP7. The crude product is purified applying semi-preparative RP-HPLC and the solvents are removed under reduced pressure. The precipitate is dissolved in tBuOH/H₂O, frozen at −80° C. and final lyophilisation yields peptide 24 as colorless solid. Due to the quaternary amine present in the peptide, the formation of a TFA-salt is assumed.

RP-HPLC semi-preparative (37-53% in 20 min): t_{R}=11.9 min.

RP-HPLC (10-60% in 15 min): t_{R}=12.2 min.

ESI-MS: m_{exact} (C₉₉H₁₄₃FN₂₁O₂₉S₂Si⁺): 2201.0; m_{found}: m/z=1101.0 [M+2H]²⁺, 1467.6 [2M+3H]³⁺, 1650.4 [3M+4H]⁴⁺.

### Synthesis of 38

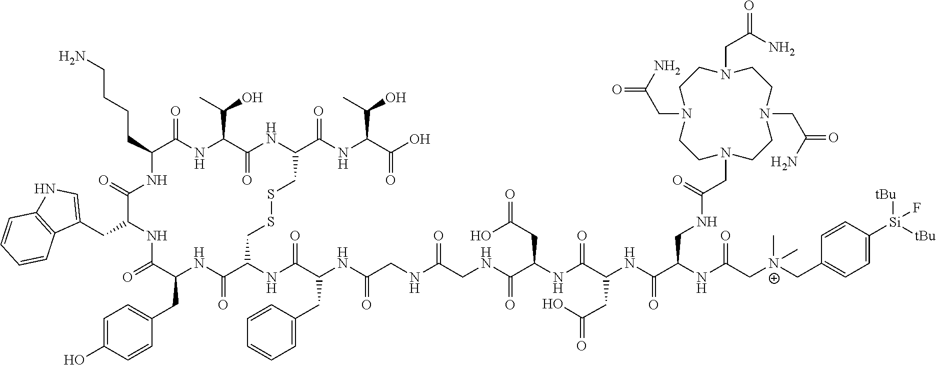

Starting from precursor X8, the synthesis of ligand 38 is carried out analogously to the synthesis described for 24. In contrast to the synthesis of 24, DO3AM-acetic acid (1.8 eq.) is used as chelator, applying HOAt (1.5 eq.), TBTU (1.5 eq.) and DIPEA (5.5 eq.) in DMF/NMP (1/1 v/v; 8 mL/g resin) for 3 h at room temperature. All other synthesis steps are transferable. After deprotection and cleavage from the resin, the crude product is purified applying semi-preparative RP-HPLC, yielding peptide 38 as colorless solid.

RP-HPLC semi-preparative (35-43% in 20 min): t_{R}=14.6 min.

RP-HPLC (10-60% in 15 min): t_{R}=˜ 11.7 min.

ESI-MS: m_{exact} (C₉₉H₁₄₆FN₂₄O₂₆S₂Si⁺): 2198.0; m_{found}: m/z=734.5 [M+3H]³⁺, 1101.3 [M+2H]²⁺.

### Synthesis of 48

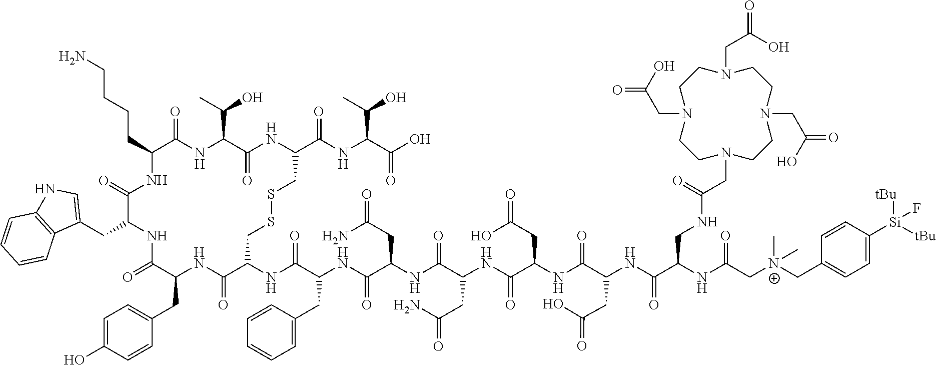

Starting from precursor X18, the synthesis of ligand 48 is carried out analogously to the synthesis described for 24. All synthesis steps are transferable. After deprotection and cleavage from the resin, the crude product is purified applying semi-preparative RP-HPLC, yielding peptide 48 as colorless solid.

RP-HPLC semi-preparative (40-48% in 20 min): t_{R}=16.0 min.

RP-HPLC (10-60% in 15 min): t_{R}=11.9 min.

ESI-MS: m_{exact} (C₁₀₃H₁₄₉FN₂₃O₃₁S₂Si⁺): 2315.0; m_{found}: m/z=772.3 [M+3H]³⁺, 1032.7 [M-CH₂—C₆H₄—SitBu₂F+2H]²⁺, 1157.8 [M+2H]²⁺.

### Synthesis of 49

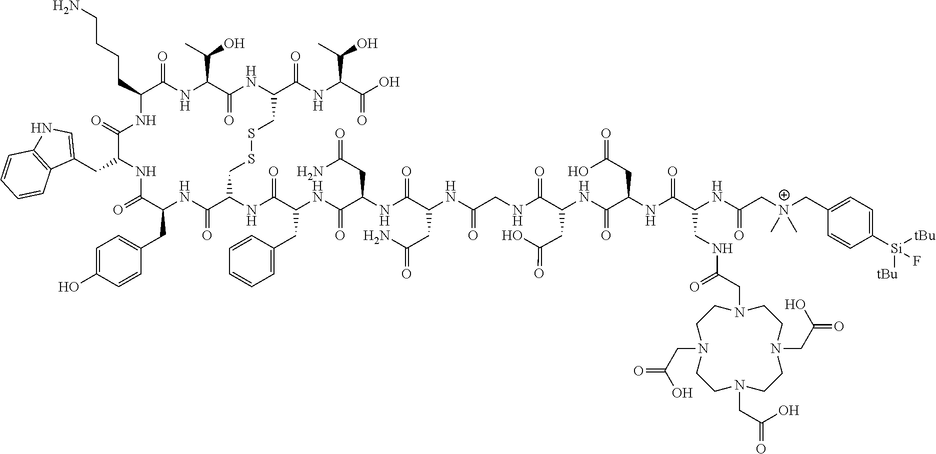

Starting from precursor X19, the synthesis of ligand 49 is carried out analogously to the synthesis described for 24. All synthesis steps are transferable. After deprotection and cleavage from the resin, the crude product is purified applying semi-preparative RP-HPLC, yielding peptide 49 as colorless solid.

RP-HPLC semi-preparative (40-45% in 20 min): t_{R}=17.0 min.

RP-HPLC (10-60% in 15 min): t_{R}=11.8 min.

ESI-MS: m_{exact} (C₁₀₅H₁₅₂FN₂₄O₃₂S₂Si⁺): 2372.0; m_{found}: m/z=791.3 [M+3H]³⁺, 1061.2 [M-CH₂—C₆H₄—SitBu₂F+2H]²⁺, 1186.4 [M+2H]²⁺.

### Synthesis of 50

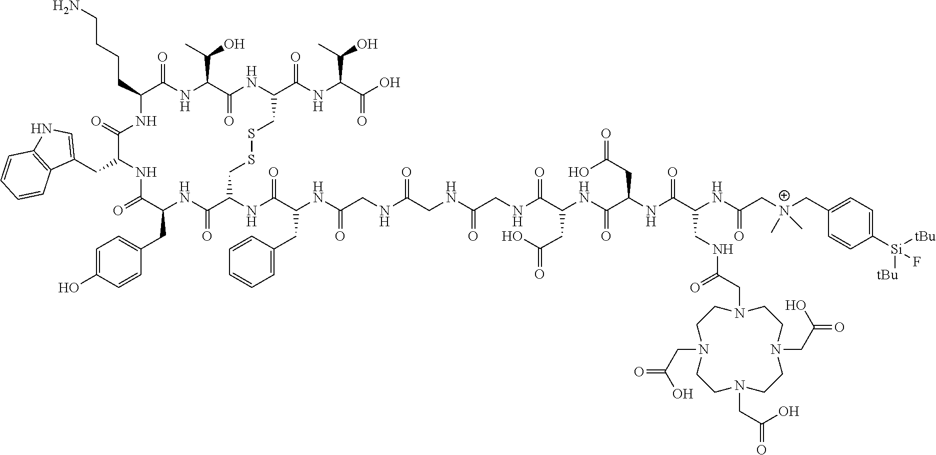

Starting from precursor X15, the synthesis of ligand 50 is carried out analogously to the synthesis described for 24. All synthesis steps are transferable. After deprotection and cleavage from the resin, the crude product is purified applying semi-preparative RP-HPLC, yielding peptide 50 as colorless solid.

RP-HPLC semi-preparative (42-47% in 20 min): t_{R}=14.1 mli.

RP-HPLC (10-60% in 15 min): t_{R}=12.0 min.

ESI-MS: m_{exact} (C101H₁₄₆FN₂₂O₃₀S₂Si⁺): 2258.0; m_{found}: m/z=753.3 [M+3H]³⁺, 1004.2 [M-CH₂—C₆H₄—SitBu₂F+2H]²⁺, 1129.4 [M+2H]²⁺.

### Synthesis of 54

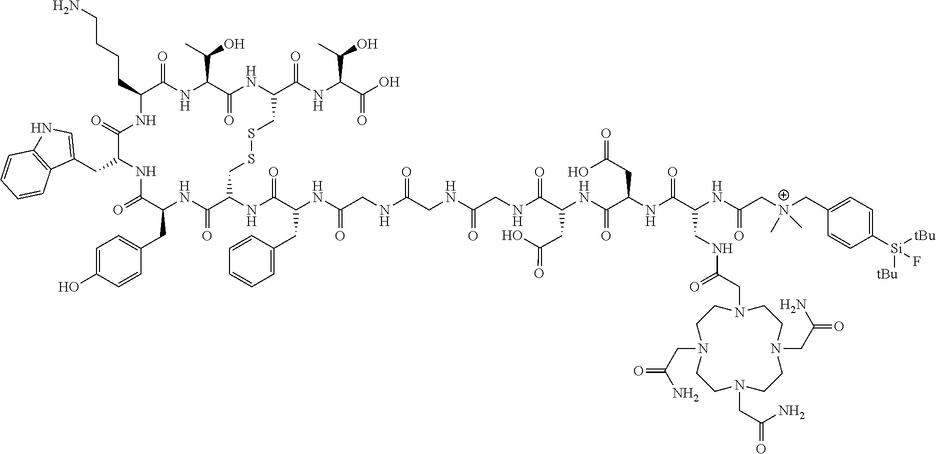

Starting from precursor X15, the synthesis of ligand 54 is carried out analogously to the synthesis described for 24. In contrast to the synthesis of 24, DO3AM-acetic acid (1.8 eq.) is used as chelator, applying HOAt (1.5 eq.), TBTU (1.5 eq.) and DIPEA (5.5 eq.) in DMF (8 mL/g resin) for 3 h at room temperature. All other synthesis steps are transferable. After deprotection and cleavage from the resin, the crude product is purified applying semi-preparative RP-HPLC, yielding peptide 54 as colorless solid.

RP-HPLC semi-preparative (36-42% in 20 min): t_{R}=11.8 min.

RP-HPLC (10-60% in 15 min): t_{R}=11.6 min.

ESI-MS: m_{exact} (C₁₀₁H₁₄₉FN₂₅O₂₇S₂Si⁺): 2255.0; m_{found}: m/z=564.8 [M+4H]⁴⁺, 752.7 [M+3H]³⁺, 1128.6 [M+2H]²⁺, 1504.8 [2M+3H]³⁺.

### Synthesis of 55

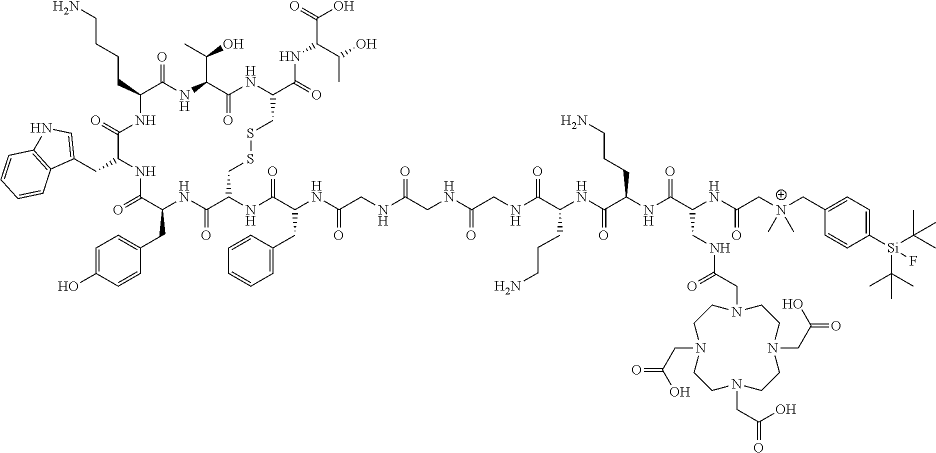

Starting from precursor X23, the synthesis of ligand 55 is carried out analogously to the synthesis described for 24. All synthesis steps are transferable. After deprotection and cleavage from the resin, the crude product is purified applying semi-preparative RP-HPLC, yielding peptide 55 as colorless solid.

RP-HPLC semi-preparative (34-45% in 20 min): t_{R}=11.9 min.

RP-HPLC (10-60% in 15 min): t_{R}=11.0 min.

ESI-MS: m_{exact} (C₁₀₃H₁₅₆FN₂₄O₂₆S₂Si⁺): 2256.1; m_{found}: m/z=565.1 [M+4H]⁴⁺, 753.2 [M+3H]³⁺, 1129.2 [M+2H]²⁺, 1505.1 [2M+3H]³⁺, 1693.8 [3M+4H]⁴⁺.

### Synthesis of 56

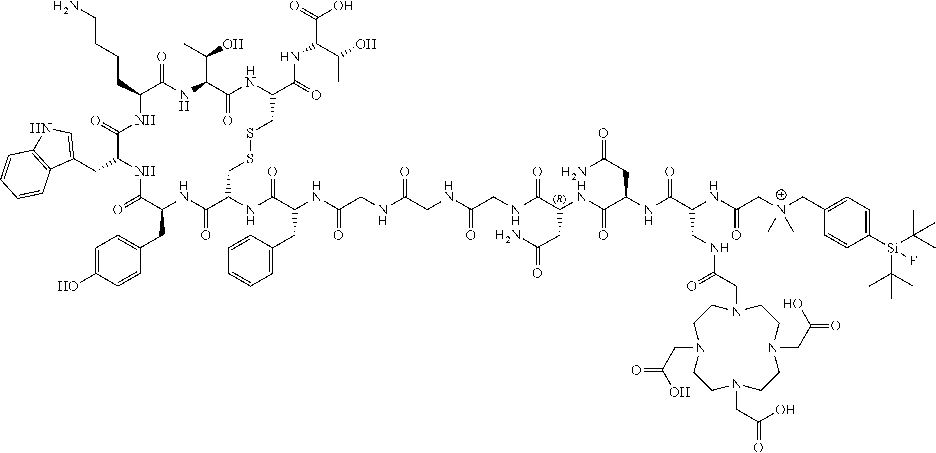

Starting from precursor X24, the synthesis of ligand 56 is carried out analogously to the synthesis described for 24. All synthesis steps are transferable. After deprotection and cleavage from the resin, the crude product is purified applying semi-preparative RP-HPLC, yielding peptide 56 as colorless solid.

RP-HPLC semi-preparative (36-48% in 20 min): t_{R}=12.6 min.

RP-HPLC (10-60% in 15 min): t_{R}=11.9 min.

ESI-MS: m_{exact} (C₁₀₁H₁₄₈FN₂₄O₂₈S₂Si⁺): 2256.0; m_{found}: m/z=753.0 [M+3H]³⁺, 1129.2 [M+2H]²⁺, 1505.3 [2M+3H]³⁺, 1694.0 [3M+4H]⁴⁺.

### Synthesis of 57

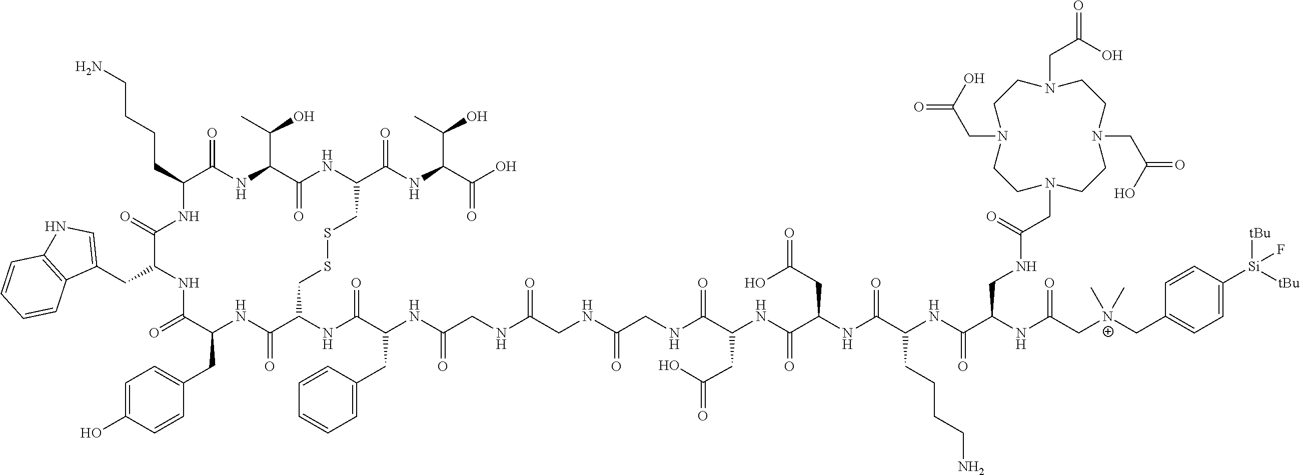

Starting from precursor X20, the synthesis of ligand 57 is carried out analogously to the synthesis described for 24. All synthesis steps are transferable. After deprotection and cleavage from the resin, the crude product is purified applying semi-preparative RP-HPLC, yielding peptide 57 as colorless solid.

RP-HPLC semi-preparative (35-42% in 20 min): t_{R}=12.3 min.

RP-HPLC (10-60% in 15 min): t_{R}=11.4 min.

ESI-MS: m_{exact} (C₁₀₇H₁₅₈FN₂₄O₃₁S₂Si⁺): 2386.1; m_{found}: m/z=2256.0; m_{found}: m/z=597.7 [M+4H]⁴⁺, 796.4 [M+3H]³⁺, 1194.2 [M+2H]²⁺, 1592.3 [2M+3H]³⁺, 1791.3 [3M+4H]⁴⁺, 1910.7 [4M+5H]⁵⁺.

### Synthesis of 58

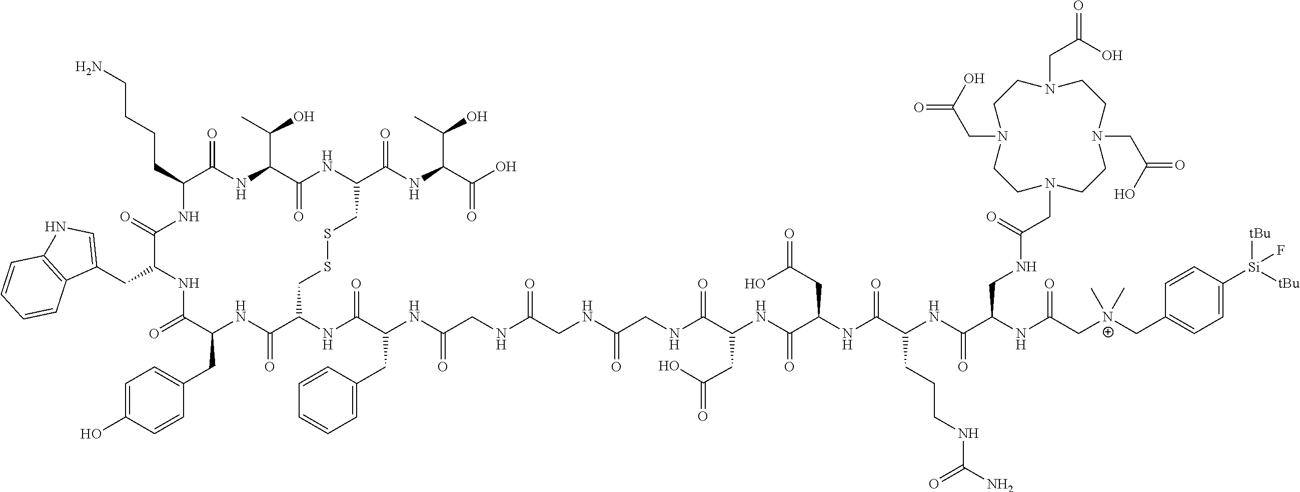

Starting from precursor X21, the synthesis of ligand 58 is carried out analogously to the synthesis described for 24. All synthesis steps are transferable. After deprotection and cleavage from the resin, the crude product is purified applying semi-preparative RP-HPLC, yielding peptide 58 as colorless solid.

RP-HPLC semi-preparative (38-42% in 20 min): t_{R}=11.3 min.

RP-HPLC (10-60% in 15 min): t_{R}=11.9 min.

ESI-MS: m_{exact} (C₁₀₇H₁₅₇FN₂₅O₃₂S₂Si⁺): 2415.1; m_{found}: m/z=806.1 [M+3H]³⁺, 1208.8 [M+2H]²⁺.

### Synthesis of 59

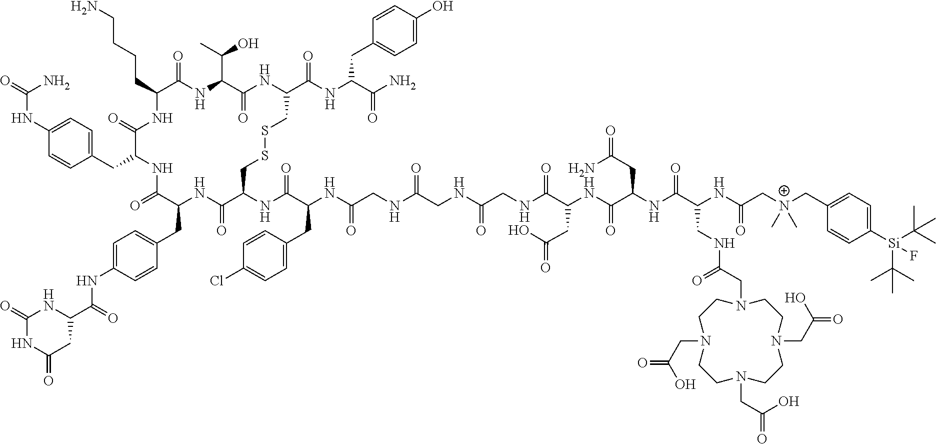

Starting from precursor X26, the synthesis of ligand 59 is carried out analogously to the synthesis described for 24. All synthesis steps are transferable. After deprotection and cleavage from the resin, the crude product is purified applying semi-preparative RP-HPLC, yielding peptide 59 as colorless solid.

RP-HPLC semi-preparative (37-45% in 20 min): t_{R}=14.1 mli.

RP-HPLC (10-60% in 15 min): t_{R}=11.9 min.

ESI-MS: m_{exact} (C₁₁₀H₁₅₄ClFN₂₇O₃₂S₂Si⁺): 2511.0; m_{found}: m/z=629.0 [M+4H]⁴⁺, 838.3 [M+3H]³⁺, 1257.0 [M+2H]²⁺, 1676.1 [2M+3H]³⁺, 1885.3 [3M+4H]⁴⁺.

### Synthesis of 61

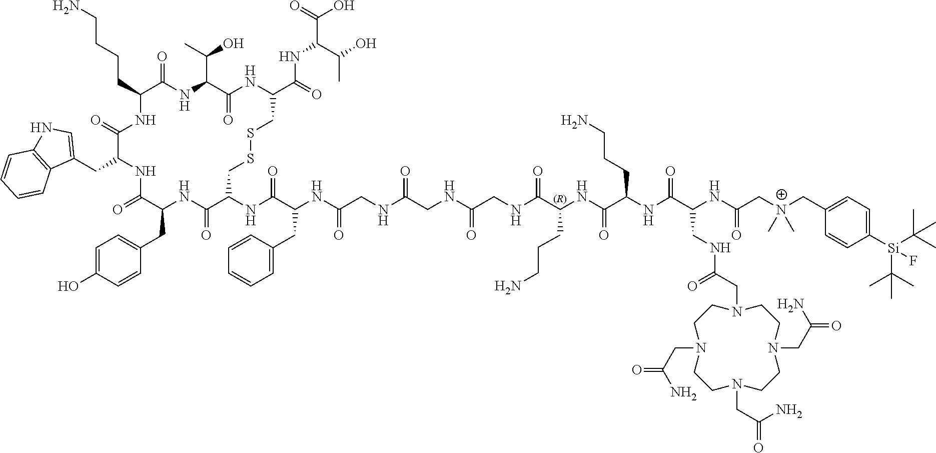

Starting from precursor X23, the synthesis of ligand 61 is carried out analogously to the synthesis described for 24. In contrast to the synthesis of 24, DO3AM-acetic acid (1.8 eq.) is used as chelator, applying HOAt (1.5 eq.), TBTU (1.5 eq.) and DIPEA (5.5 eq.) in DMF (8 mL/g resin) for 3 h at room temperature. All other synthesis steps are transferable. After deprotection and cleavage from the resin, the crude product is purified applying semi-preparative RP-HPLC, yielding peptide 61 as colorless solid.

RP-HPLC semi-preparative (33-45% in 20 min): t_{R}=11.6 min.

RP-HPLC (10-60% in 15 min): t_{R}=10.6 min.

ESI-MS: m_{exact} (C₁₀₃H₁₅₉FN₂₇O₂₃S₂Si⁺): 2253.1; m_{found}: m/z=564.5 [M+4H]⁴⁺, 752.2 [M+3H]³⁺, 1127.4 [M+2H]²⁺.

### Synthesis of 44

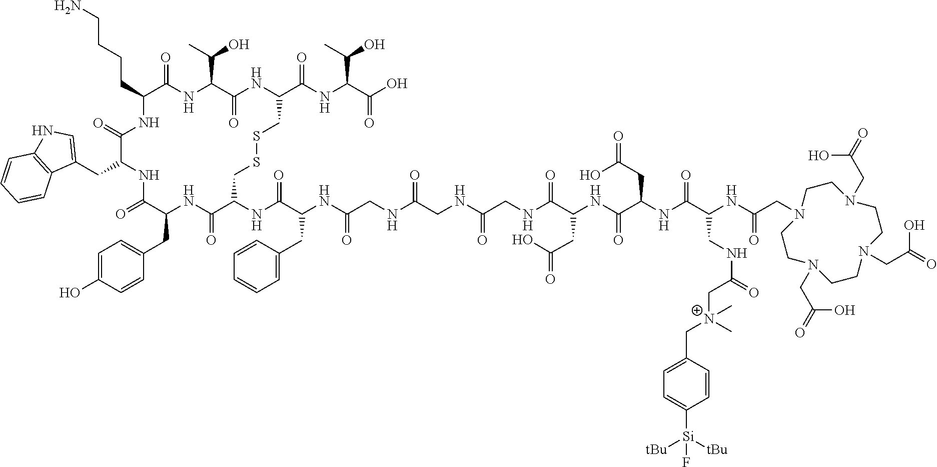

The synthesis of the ligand 44 is carried out applying GP2, GP3, GP4, GP5 and GP7. Briefly, resin bound precursor X15 (1.0 eq.) is conjugated with Fmoc-D-Dap(Dde)-OH (1.5 eq.) applying HOAt (1.5 eq.) TBTU (1.5 eq.) and 2,4,6-collidine (5.5 eq.). After Dde-deprotection, Me₂-Gly-OH (3.0 eq.) is conjugated, applying HOAt (3.0 eq.), TBTU (3.0 eq.) and 2,4,6-collidine (9.0 eq.) in DMF (8 mL/g resin) for 4 h at room temperature. The N-terminus is Fmoc-deprotected and conjugated with DOTA(tBu)₃ (1.5 eq), applying HOAt (1.5 eq.), HATU (1.5 eq.) and DIPEA (4.5 eq.). Subsequently the SiFA-moiety is formed, by incubation with SiFA-Br (3.0 eq.) and 2,4,6-collidine (6.0 eq.) in DCM (8 mL/g resin) for 24 h at room temperature. Reaction control via test cleavage (GP8; mild test cleavage) confirms correct but incomplete product formation. Nevertheless, the resin bound peptide is deprotected and cleaved from the resin by treatment with TFA/TIPS/H₂O, as described in GP7. The crude product is purified applying semi-preparative RP-HPLC and the solvents are removed under reduced pressure. The precipitate is dissolved in tBuOH/H₂O, frozen at −80° C. and final lyophilisation yields peptide 44 as colorless solid. Due to the quaternary amine present in the peptide, the formation of a TFA-salt is assumed.

RP-HPLC semi-preparative (35-45% in 20 min): t_{R}=13.2 min.

RP-HPLC (10-60% in 15 min): t_{R}=12.0 min.

ESI-MS: m_{exact} (C₁₀₁H₁₄₆FN₂₂O₃₀S₂Si⁺): 2258.0; m_{found}: m/z=753.2 [M+3H]³⁺, 1129.2 [M+2H]²⁺, 1505.0 [2M+3H]³⁺.

### Synthesis of 45

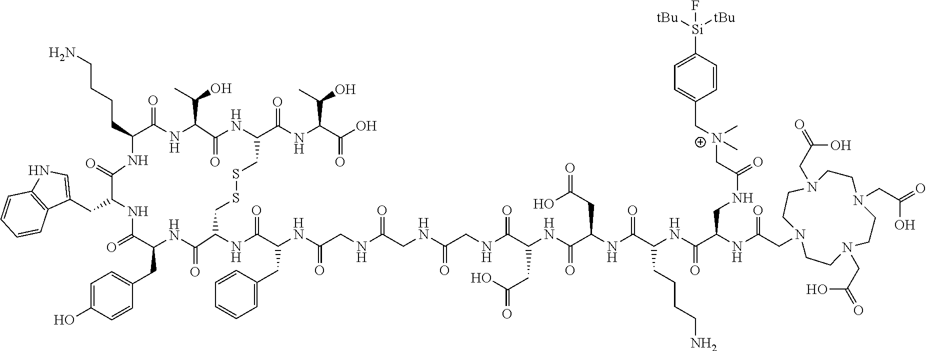

Starting from precursor X20, the synthesis of ligand 45 is carried out analogously to the synthesis described for 44. All synthesis steps are transferable. After deprotection and cleavage from the resin, the crude product is purified applying semi-preparative RP-HPLC, yielding peptide 45 as colorless solid.

RP-HPLC semi-preparative (32-42% in 20 min): t_{R}=15.0 min.

RP-HPLC (10-60% in 15 min): t_{R}=11.3 min.

ESI-MS: m_{exact} (C₁₀₇H₁₅₈FN₂₄O₃₁S₂Si⁺): 2386.1; m_{found}: m/z=597.4 [M+4H]⁴⁺, 795.8 [M+3H]³⁺, 1193.2 [M+2H]²⁺, 1590.7 [2M+3H]³⁺.

### Synthesis of 46

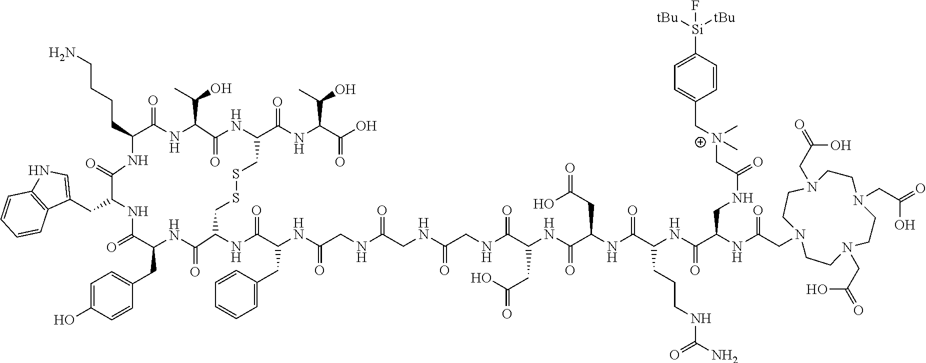

Starting from precursor X21, the synthesis of ligand 46 is carried out analogously to the synthesis described for 44. All synthesis steps are transferable. After deprotection and cleavage from the resin, the crude product is purified applying semi-preparative RP-HPLC, yielding peptide 46 as colorless solid.

RP-HPLC semi-preparative (35-42% in 20 min): t_{R}=12.1 min.

RP-HPLC (10-60% in 15 min): t_{R}=11.8 min.

ESI-MS: m_{exact} (C₁₀₇H₁₅₇FN₂₅O₃₂S₂Si⁺): 2415.1; m_{found}: m/z=805.4 [M+3H]³⁺, 1207.7 [M+2H]²⁺, 1610.0 [2M+3H]³⁺

### Synthesis of 47

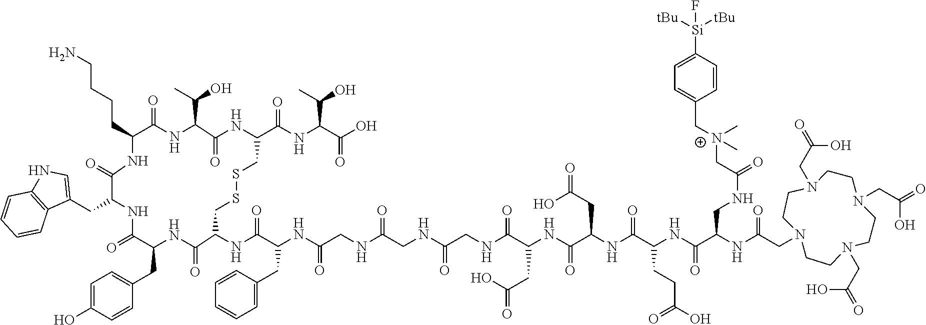

Starting from precursor X22, the synthesis of ligand 47 is carried out analogously to the synthesis described for 44. All synthesis steps are transferable. After deprotection and cleavage from the resin, the crude product is purified applying semi-preparative RP-HPLC, yielding peptide 47 as colorless solid.

RP-HPLC semi-preparative (40-50% in 20 min): t_{R}=15.5 min.

RP-HPLC (10-60% in 15 min): t_{R}=11.9 min.

ESI-MS: m_{exact} (C₁₀₆H₁₅₃FN₂₃O₃₃S₂Si⁺): 2387.0; m_{found}: m/z=796.3 [M+3H]³⁺, 1068.7 [M-CH₂—C₆H₄—SitBu₂F+2H]²⁺, 1193.8 [M+2H]²⁺.

### Synthesis of 51

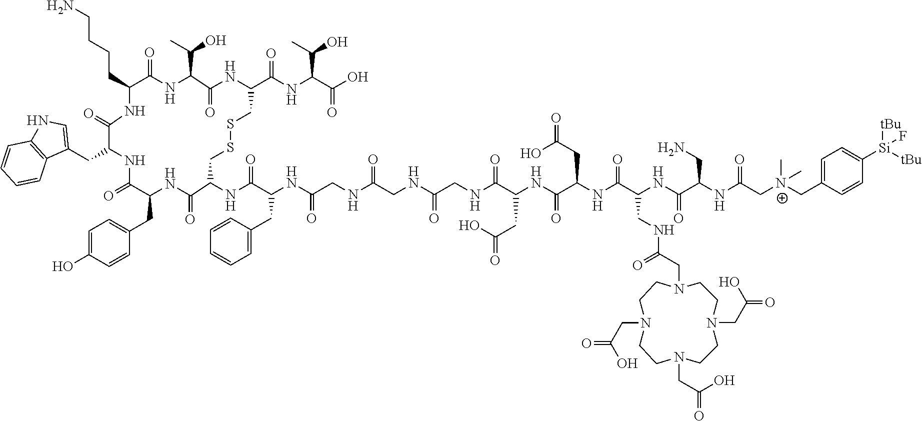

The synthesis of the ligand 51 is carried out applying GP2, GP3, GP4, GP5 and GP7. Briefly, resin bound precursor X15 (1.0 eq.) is conjugated with Fmoc-D-Dap(Dde)-OH (1.5 eq.) applying HOAt (1.5 eq.) TBTU (1.5 eq.) and 2,4,6-collidine (5.5 eq.). After Dde-deprotection, DOTA(tBu)₃ (1.5 eq.) is conjugated, applying HOAt (1.5 eq.), HATU (1.5 eq.) and DIPEA (4.5 eq.). The N-terminus is Fmoc-deprotected and conjugated with Fmoc-D-Dap(Boc)-OH (1.5 eq.) applying HOAt (1.5 eq.) TBTU (1.5 eq.) and 2,4,6-collidine (5.5 eq.). After Fmoc-deprotection Me₂-Gly-OH (3.0 eq.) is conjugated, applying HOAt (3.0 eq.), TBTU (3.0 eq.) and 2,4,6-collidine (9.0 eq.) in DMF (8 mL/g resin) for 4 h at room temperature. The SiFA-moiety is formed, by incubation with SiFA-Br (3.0 eq.) and 2,4,6-collidine (6.0 eq.) in DCM (8 mL/g resin) for 24 h at room temperature. Reaction control via test cleavage (GP8; mild test cleavage) confirms correct but incomplete product formation. Nevertheless, the resin bound peptide is deprotected and cleaved from the resin by treatment with TFA/TIPS/H₂O, as described in GP7. The crude product is purified applying semi-preparative RP-HPLC and the solvents are removed under reduced pressure. The precipitate is dissolved in tBuOH/H₂O, frozen at −80° C. and final lyophilisation yields peptide 51 as colorless solid. Due to the quaternary amine present in the peptide, the formation of a TFA-salt is assumed.

RP-HPLC semi-preparative (40-45% in 20 min): t_{R}=14.5 min.

RP-HPLC (10-60% in 15 min): t_{R}=11.6 min.

ESI-MS: m_{exact} (C₁₀₄H₁₅₂FN₂₄O₃₁S₂Si⁺): 2344.0; m_{found}: m/z=781.3 [M+3H]³⁺, 1171.4 [M+2H]²⁺, 1156.9 [2M+3H]³⁺, 1757.1 [3M+4H]⁴⁺.

### Synthesis of 60

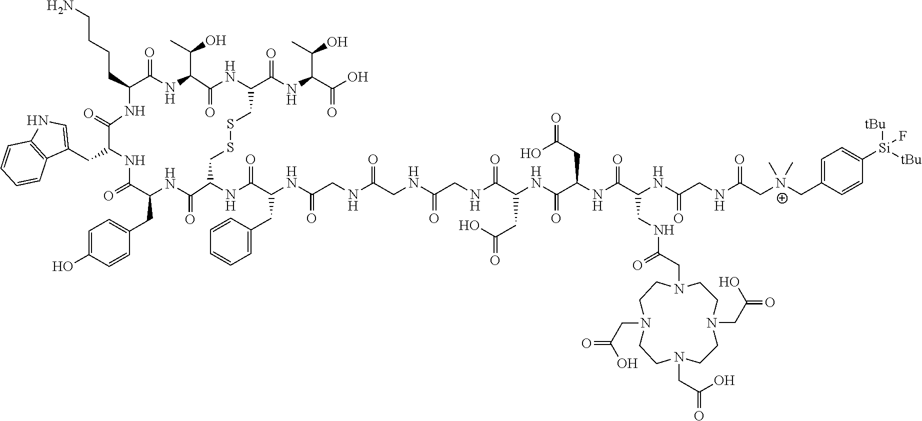

Starting from precursor X15, the synthesis of ligand 60 is carried out analogously to the synthesis described for 51. In contrast to the synthesis of 51, Fmoc-Gly-OH is conjugated instead of Fmoc-D-Dap(Boc)-OH, applying HOAt (1.5 eq.), TBTU (1.5 eq.) and DIPEA (4.5 eq.). All other synthesis steps are transferable. After deprotection and cleavage from the resin, the crude product is purified applying semi-preparative RP-HPLC, yielding peptide 60 as colorless solid.

RP-HPLC semi-preparative (37-43% in 20 min): t_{R}=15.8 min.

RP-HPLC (10-60% in 15 min): t_{R}=12.2 min.

ESI-MS: m_{exact} (C₁₀₃H₁₄₉FN₂₃O₃₁S₂Si⁺): 2315.0; m_{found}: m/z=579.8 [M+4H]⁴⁺, 772.8 [M+3H]³⁺, 1158.5 [M+2H]²⁺, 1544.9 [2M+3H]³⁺.

### Synthesis of 52

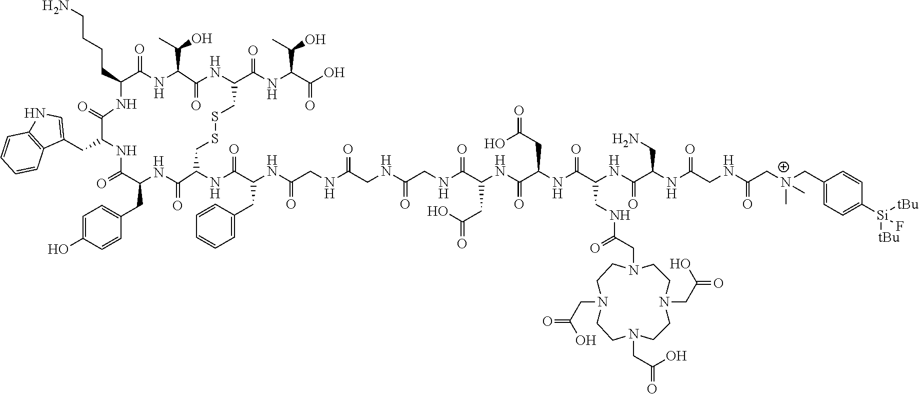

Starting from precursor X15, the synthesis of ligand 52 is carried out analogously to the synthesis described for 51. In contrast to the synthesis of 51, Fmoc-Gly-OH is coupled before the conjugation of Me₂-Gly-OH. All other synthesis steps are transferable. After deprotection and cleavage from the resin, the crude product is purified applying semi-preparative RP-HPLC, yielding peptide 52 as colorless solid.

RP-HPLC semi-preparative (45-48% in 20 min): t_{R}=11.3 min.

RP-HPLC (10-60% in 15 min): t_{R}=11.7 min.

ESI-MS: m_{exact} (C₁₀₆H₁₅₅FN₂₅O₃₂S₂Si⁺): 2401.1; m_{found}: m/z=800.2 [M+3H]³⁺, 1199.9 [M+2H]²⁺, 1599.9 [2M+3H]³⁺, 1800.2 [3M+4H]⁴⁺.

### Synthesis of 53

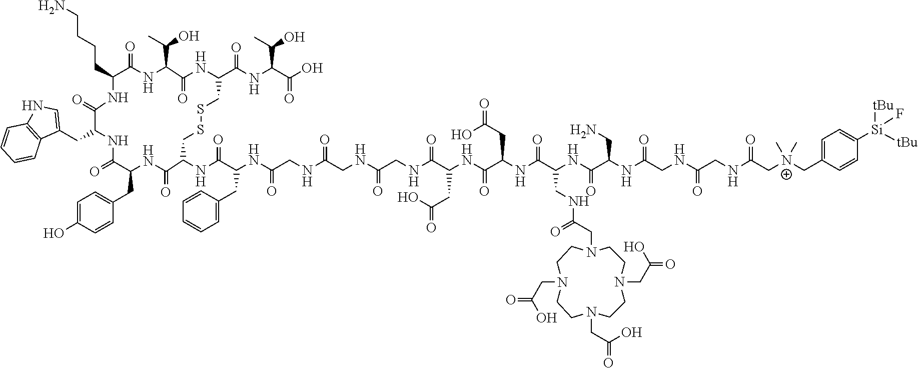

Starting from precursor X15, the synthesis of ligand 53 is carried out analogously to the synthesis described for 51. In contrast to the synthesis of 51, Fmoc-Gly-OH is coupled twice before the conjugation of Me₂-Gly-OH. All other synthesis steps are transferable. After deprotection and cleavage from the resin, the crude product is purified applying semi-preparative RP-HPLC, yielding peptide 53 as colorless solid.

RP-HPLC semi-preparative (42-45% in 20 min): t_{R}=13.5 min.

RP-HPLC (10-60% in 15 min): t_{R}=11.8 min.

ESI-MS: m_{exact} (C₁₀₈H₁₅₈FN₂₆O₃₃S₂Si⁺): 2458.1; m_{found}: m/z=819.2 [M+3H]³⁺, 1228.3 [M+2H]²⁺, 1637.4 [2M+3H]³⁺, 1841.8 [3M+4H]⁴.

### 3.4.5 Metal Complexation for Biological Evaluation

^{nat}Ga^{III}-, ^{nat}Lu^{III}- and ^{nat}Pb^{II}-chelate formation was achieved applying general procedures GP9, GP10 and GP11. The resulting analytical data (analytical RP-HPLC and ESI-MS) are listed below.

^{nat}Ga^{III}-Complexes
[^{nat}Ga]1

RP-HPLC (10-90% in 15 min): t_{R}=10.9 min.

ESI-MS: m_{exact} (C₁₀₀H₁₃₉FN₁₉O₃₂S₂SiGa): 2298.7; m_{found}: m/z=767.6 [M+3H]³⁺, 1150.7 [M+2H]²⁺, 1533.9 [2M+3H]³⁺.

[^{nat}Ga]2

RP-HPLC (10-90% in 15 min): t_{R}=10.4 min.

ESI-MS: m_{exact} (C₁₀₀H₁₃₉FN₁₉O₃₂S₂SiGa): 2298.7; m_{found}: m/z=767.6 [M+3H]³⁺, 1150.7 [M+2H]²⁺, 1533.9 [2M+3H]³⁺.

[^{nat}Ga]3

RP-HPLC (10-90% in 15 min): t_{R} 11.1 min.

ESI-MS: m_{exact} (C₉₇H₁₃₅FN₁₉O₃₀S₂SiGa): 2226.7; m_{found}: m/z=743.3 [M+3H]³⁺, 1114.6 [M+2H]²⁺, 1485.9 [2M+3H]³⁺.

[^{nat}Ga]4

RP-HPLC (10-90% in 15 min): t_{R}=11.4 min.

ESI-MS: m_{exact} (C₉₇H₁₃₅FN₁₉O₃₀S₂SiGa): 2226.7; m_{found}: m/z=743.3 [M+3H]³⁺, 1114.6 [M+2H]²⁺, 1485.9 [2M+3H]³⁺.

[^{nat}Ga]5

RP-HPLC (10-90% in 15 min): t_{R}=8.87 min.

ESI-MS: m_{exact} (C₁₀₉H₁₄₅FN₂₁O₃₆S₂SiGa): 2504.7; m_{found}: m/z=836.2 [M+3H]³⁺, 1253.5 [M+2H]²⁺, 1671.4 [2M+3H]³⁺.

[^{nat}Ga]6

RP-HPLC (10-90% in 15 min): t_{R}=8.81 min.

ESI-MS: m_{exact} (C₉₈H₁₃₄FN₂₀O₃₁S₂SiGa): 2267.6; m_{found}: m/z=1135.6 [M+2H]²⁺, 1513.6 [2M+3H]³⁺.

[^{nat}Ga]7

RP-HPLC (10-90% in 15 min): t_{R}=8.77 min.

ESI-MS: m_{exact} (C₉₅H₁₃₄FN₂₀O₃₁S₂SiGa): 2267.66; m_{found}: m/z=1135.6 [M+2H]²⁺, 1513.6 [2M+3H]³⁺.

[^{nat}Ga]8

RP-HPLC (10-90% in 15 min): t_{R}=8.80 min.

ESI-MS: m_{exact} (C₉₅H₁₃₀FN₂₀O₂₉S₂SiGa): 2195.6; m_{found}: m/z=1099.6 [M+2H]²⁺, 1466.4 [2M+3H]³⁺

[^{nat}Ga]9

RP-HPLC (10-90% in 15 min): t_{R}=8.81 min.

ESI-MS: m_{exact} (C₉₅H₁₃₀FN₂₀O₂₉S₂SiGa): 2195.6; m_{found}: m/z=1099.6 [M+2H]²⁺, 1466.4 [2M+3H]³⁺.

[^{nat}Ga]18

RP-HPLC (10-60% in 15 min): t_{R}=11.6 min.

ESI-MS: m_{exact} (C₁₀₀H₁₄₅FGaN₂₂O₂₇S₂Si): 2265.9; m_{found}: m/z=1134.6 [M+2H]²⁺, 757.0 [M+3H]³⁺.

[^{nat}Ga]19

RP-HPLC (10-60% in 15 min): t_{R}=11.7 min.

ESI-MS: m_{exact} (C₉₆H₁₃₈FGaN₂₂O₃₀S₂Si): 2282.8; m_{found}: m/z=1142.1 [M+2H]²⁺, 1522.5 [2M+3H]³⁺, 1712.1 [3M+4H]⁴⁺.

[^{nat}Ga]20

RP-HPLC (10-60% in 15 min): t_{R}=12.0 min.

ESI-MS: m_{exact} (C₁₀₁H₁₄₁FGaN₂₂O₃₂S₂Si): 2353.9; m_{found}: m/z=1178.2 [M+2H]²⁺, 1570.5 [2M+3H]³⁺, 1766.4 [3M+4H]⁴⁺.

[^{nat}Ga]23

RP-HPLC (10-60% in 15 min): t_{R}=12.0 min.

ESI-MS: m_{exact} (C₁₀₀H₁₄₂FGaN₂₁O₃₁S₂Si): 2312.9; m_{found}: m/z=771.9 [M+3H]³⁺, 1157.6 [M+2H]²⁺, 1542.9 [2M+3H]³⁺.

[^{nat}Ga]24

RP-HPLC (10-60% in 15 min): t_{R}=12.4 min.

ESI-MS: m_{exact} (C₉₉H₁₄₁FGaN₂₁O₂₉S₂Si⁺): 2267.9; m_{found}: m/z=756.6 [M+3H]³⁺, 1134.4 [M+2H]²⁺, 1512.6 [2M+3H]³⁺.

[^{nat}Ga]37

RP-HPLC (10-60% in 15 min): t_{R}=11.1 min.

ESI-MS: m_{exact} (C₁₀₃H₁₁FGaN₂₄O₂₈S₂Si): 2352.0; m_{found}: m/z=589.2 [M+4H]⁴⁺, 785.4 [M+3H]³⁺, 1177.6 [M+2H]²⁺. [^{nat}Ga]39

RP-HPLC (10-60% in 15 min): t_{R}=12.0 min.

ESI-MS: m_{exact} (C₉₆H₁₃₄FGaN₂₁O₂₉S₂Si): 2224.8; m_{found}: m/z=743.0 1114.1. [^{nat}Ga]40

RP-HPLC (10-60% in 15 min): t_{R}=11.8 min.

ESI-MS: m_{exact} (C₁₀₀H₁₄₀FGaN₂₃O₃₁S₂Si): 2338.9; m_{found}: m/z=781.1 [M+3H]³⁺, 1171.1 [M+2H]²⁺, 1561.3 [2M+3H]³⁺.

[^{nat}Ga]41

RP-HPLC (10-60% in 15 min): t_{R}=12.0 min.

ESI-MS: m_{exact} (C₁₀₁H₁₄₃FGaN₂₄O₃₀S₂Si): 2351.9; m_{found}: m/z=785.2 [M+3H]³⁺, 1177.7 [M+2H]²⁺, 1571.0 [2M+3H]³⁺.

[^{nat}Ga]42

RP-HPLC (10-60% in 15 min): t_{R}=11.7 min.

ESI-MS: m_{exact} (C₁₀₂H₁₄₃FGaN₂₄O₃₂S₂Si): 2395.9; m_{found}: m/z=800.2 [M+3H]³⁺, 1200.3 [M+2H]²⁺, 1599.2 [2M+3H]³⁺.

[^{nat}Ga]43

RP-HPLC (10-60% in 15 min): t_{R} min.

ESI-MS: m_{exact} (C₁₀₂H₁₄₃FGaN₂₄O₃₂S₂Si): 2395.9; m_{found}: m/z=799.8 [M+3H]³⁺, 1199.5 [M+2H]²⁺, 1599.8 [2M+3H]³⁺.

[^{nat}Ga]44

RP-HPLC (10-60% in 15 min): t_{R}=12.1 min.

ESI-MS: m_{exact} (C₁₀₁H₁₄₄FGaN₂₂O₃₀S₂Si⁺): 2324.9; m_{found}: m/z=775.4 [M+3H]³⁺, 1162.4 [M+2H]²⁺, 1549.6 [2M+3H]³⁺.

[^{nat}Ga]45

RP-HPLC (10-60% in 15 min): t_{R}=11.4 min.

ESI-MS: m_{exact} (C₁₀₇H₁₅₆FGaN₂₄O₃₁S₂Si⁺): 2453.0; m_{found}: m/z=817.9 [M+3H]³⁺, 1226.0 [M+2H]²⁺, 1634.6 [2M+3H]³⁺.

[^{nat}Ga]46

RP-HPLC (10-60% in 15 min): t_{R}=11.9 min.

ESI-MS: m_{exact} (C₁₀₇H₁₅₅FGaN₂₅O₃₂S₂Si⁺): 2482.0; m_{found}: m/z=827.6 [M+3H]³⁺, 1240.8 [M+2H]²⁺, 1653.3 [2M+3H]³⁺.

[^{nat}Ga]47

RP-HPLC (10-60% in 15 min): t_{R}=12.0 min.

ESI-MS: m_{exact} (C₁₀₆H₁₁FGaN₂₃O₃₃S₂Si⁺): 2453.9; m_{found}: m/z=817.9 [M+3H]³⁺, 1226.6 [M+2H]²⁺, 1635.3 [2M+3H]³⁺.

[^{nat}Ga]48

RP-HPLC (10-60% in 15 min): t_{R}=12.1 min.

ESI-MS: m_{exact} (C₁₀₃H₁₄₇FGaN₂₃O₃₁S₂Si⁺): 2381.9; m_{found}: m/z=794.1 [M+3H]³⁺, 1190.3 [M+2H]²⁺, 1587.6 [2M+3H]³⁺.

[^{nat}Ga]49

RP-HPLC (10-60% in 15 min): t_{R}=12.0 min.

ESI-MS: m_{exact} (C₁₀₅H₁₅₀FGaN₂₄O₃₂S₂Si⁺): 2438.9; m_{found}: m/z=813.1 [M+3H]³⁺, 1218.8 [M+2H]²⁺, 1625.1 [2M+3H]³⁺, 1829.4 [3M+4H]⁴⁺.

[^{nat}Ga]50

RP-HPLC (10-60% in 15 min): t_{R}=12.2 min.

ESI-MS: m_{exact} (C₁₀₁H₁₄₄FGaN₂₂O₃₀S₂Si⁺): 2324.9; m_{found}: m/z=775.1 [M+3H]³⁺, 1162.1 [M+2H]²⁺, 1549.1 [2M+3H]³⁺, 1742.3 [3M+4H]⁴⁺.

[^{nat}Ga]51

RP-HPLC (10-60% in 15 min): t_{R}=11.4 min.

ESI-MS: m_{exact} (C₁₀₄H₁₅₀FGaN₂₄O₃₁S₂Si⁺): 2410.9; m_{found}: m/z=803.5 [M+3H]3, 1204.6 [M+2H]²⁺, 1605.6 [2M+3H]³⁺.

[^{nat}Ga]52

RP-HPLC (10-60% in 15 min): t_{R}=11.5 min.

ESI-MS: m_{exact} (C₁₀₆H₁₅₃FGaN₂₅O₃₂S₂Si⁺): 2468.0; m_{found}: m/z=822.3 [M+3H]³⁺, 1232.8 [M+2H]²⁺, 1643.7 [2M+3H]³⁺.

[^{nat}Ga]53

RP-HPLC (10-60% in 15 min): t_{R}=11.9 min.

ESI-MS: m_{exact} (C₁₀₈H₁₅₆FGaN₂₆O₃₃S₂Si⁺): 2525.0; m_{found}: m/z=841.4 [M+3H]³⁺, 1261.3 [M+2H]²⁺, 1680.8 [2M+3H]³⁺.

[^{nat}Ga]55

RP-HPLC (10-60% in 15 min): t_{R}=11.1 min.

ESI-MS: m_{exact} (C₁₀₃H₁₅₄FGaN₂₄O₂₆S₂Si⁺): 2323.0; m_{found}: m/z=581.8 [M+4H]⁴⁺, 775.3 [M+3H]³⁺, 1163.2 [M+2H]²⁺.

[^{nat}Ga]56

RP-HPLC (10-60% in 15 min): t_{R}=12.0 min.

ESI-MS: m_{exact} (C₁₀₁H₁₄₆FGaN₂₄O₂₈S₂Si⁺): 2322.9; m_{found}: m/z=775.3 [M+3H]³⁺, 1161.9 [M+2H]²⁺, 1549.4 [2M+3H]³⁺.

[^{nat}Ga]57

RP-HPLC (10-60% in 15 min): t_{R}=11.4 min.

ESI-MS: m_{exact} (C₁₀₇H₁₅₆FGaN₂₄O₃₁S₂Si⁺): 2453.0; m_{found}: m/z=818.8 [M+3H]³⁺, 1227.9 [M+2H]²⁺.

[^{nat}Ga]58

RP-HPLC (10-60% in 15 min): t_{R}=12.0 min.

ESI-MS: m_{exact} (C₁₀₇H₁₅₅FGaN₂₅O₃₂S₂Si⁺): 2482.0; m_{found}: m/z=828.2 [M+3H]³⁺, 1242.2 [M+2H]²⁺, 1655.7 [2M+3H]³⁺.

[^{nat}Ga]59

RP-HPLC (10-60% in 15 min): t_{R}=12.0 min.

ESI-MS: m_{exact} (C₁₁₀H₁₅₂ClFGaN₂₇O₃₂S₂Si⁺): 2577.9; m_{found}: m/z=860.6 [M+3H]3, 1290.4 [M+2H]²⁺, 1720.9 [2M+3H]³⁺, 1935.3 [3M+4H]⁴⁺.

[^{nat}Ga]60

RP-HPLC (10-60% in 15 min): t_{R} 12.2 min.

ESI-MS: m_{exact} (C₁₀₃H₁₄₇FGaN₂₃O₃₁S₂Si⁺): 2381.9; m_{found}: m/z=795.0 [M+3H]³⁺, 1192.0 [M+2H]²⁺, 1588.6 [2M+3H]³⁺.

^{nat}Lu^{III}-Complexes
[^{nat}Lu]19

RP-HPLC (10-60% in 15 min): t_{R}=12.0 min.

ESI-MS: m_{exact} (C₉₈H₁₃₆FLuN₂₂O₃₀S₂Si): 2386.8; m_{found}: m/z=796.9 [M+3H]³⁺, 1194.6 [M+2H]²⁺, 1593.2 [2M+3H]³⁺.

[^{nat}Lu]20

RP-HPLC (10-60% in 15 min): t_{R}=12.3 min.

ESI-MS: m_{exact} (C₁₀₁H₁₄₀FLuN₂₂O₃₂S₂Si): 2458.9; m_{found}: m/z=820.8 [M+3H]³⁺, 1230.9 [M+2H]²⁺, 1641.4 [2M+3H]³⁺.

[^{nat}Lu]24

RP-HPLC (10-60% in 15 min): t_{R}=12.4 min.

ESI-MS: m_{exact} (C₉₉H₁₄₀FLuN₂₁O₂₉S₂Si⁴): 2372.9; m_{found}: m/z=790.7 [M+3H]³⁺, 1185.8 [M+2H]²⁺, 1581.2 [2M+3H]³⁺.

[^{nat}Lu]44

RP-HPLC (10-60% in 15 min): t_{R}=12.2 min.

ESI-MS: m_{exact} (C₁₀₁H₁₄₃FLuN₂₂O₃OS₂Si⁺): 2429.9; m_{found}: m/z=809.9 [M+3H]³⁺, 1214.2 [M+2H]²⁴, 1618.7 [2M+3H]³⁺.

[^{nat}Lu]45

RP-HPLC (10-60% in 15 min): t_{R}=11.6 min.

ESI-MS: m_{exact} (C₁₀₇H₁₅₅FLuN₂₄O₃₁S₂Si⁺): 2558.0; m_{found}: m/z=852.6 [M+3H]³⁺, 1278.3 [M+2H]²⁺, 1703.4 [2M+3H]³⁺.

[^{nat}Lu]46

RP-HPLC (10-60% in 15 min): t_{R}=12.0 min.

ESI-MS: m_{exact} (C₁₀₇H₁₅₄FLuN₂₅O₃₂S₂Si⁺): 2587.0; m_{found}: m/z=862.2 [M+3H]³⁺, 1292.8 [M+2H]²⁺, 1722.8 [2M+3H]³⁺.

[^{nat}Lu]47

RP-HPLC (10-60% in 15 min): t_{R}=12.1 min.

ESI-MS: m_{exact} (C₁₀₆H₁₅₀FLuN₂₃O₃₃S₂Si⁺): 2558.9; m_{found}: m/z=853.0 [M+3H]³⁺, 1278.7 [M+2H]²⁺, 1704.1 [2M+3H]³⁺.

[^{nat}Lu]48

RP-HPLC (10-60% in 15 min): t_{R}=12.1 min.

ESI-MS: m_{exact} (C₁₀₃H₁₄₆FLuN₂₃O₃₁S₂Si⁺): 2486.9; m_{found}: m/z=829.0 [M+3H]³⁺, 1242.7 [M+2H]²⁺, 1656.3 [2M+3H]³⁺.

[^{nat}Lu]49

RP-HPLC (10-60% in 15 min): t_{R}=12.0 min.

ESI-MS: m_{exact} (C₁₀₅H₁₄₉FLuN₂₄O₃₂S₂Si⁺): 2543.9; m_{found}: m/z=847.9 [M+3H]³⁺, 1271.2 [M+2H]²⁺, 1694.7 [2M+3H]³⁺, 1907.3 [3M+4H]⁴⁺.

[^{nat}Lu]50

RP-HPLC (10-60% in 15 min): t_{R}=12.3 min.

ESI-MS: m_{exact} (C₁₀₁H₁₄₃FLuN₂₂O₃₀S₂Si⁺): 2429.9; m_{found}: m/z=810.0 [M+3H]³⁺, 1214.0 [M+2H]²⁺, 1618.3 [2M+3H]³⁺.

[^{nat}Lu]51

RP-HPLC (10-60% in 15 min): t_{R}=11.9 min.

ESI-MS: m_{exact} (C₁₀₄H₁₄₉FLuN₂₄O₃₁S₂Si⁺): 2515.9; m_{found}: m/z=838.4 [M+3H]³⁺, 1257.5 [M+2H]²⁺, 1676.2 [2M+3H]³⁺.

[^{nat}Lu]52

RP-HPLC (10-60% in 15 min): t_{R}=11.9 min.

ESI-MS: m_{exact} (C₁₀₆H₁₅₂FLuN₂₅O₃₂S₂Si⁺): 2573.0; m_{found}: m/z=857.3 [M+3H]³⁺, 1285.0 [M+2H]²⁺, 1714.6 [2M+3H]³⁺.

[^{nat}Lu]53

RP-HPLC (10-60% in 15 min): t_{R}=12.3 min.

ESI-MS: m_{exact} (C₁₀₈H₁₅₅FLuN₂₆O₃₃S₂Si⁺): 2630.0; m_{found}: m/z=876.4 [M+3H]³⁺, 1314.2 [M+2H]²⁺, 1751.9 [2M+3H]³⁺.

^{nat}Pb^{II}-Complexes
[^{nat}Pb]36

RP-HPLC (10-60% in 15 min): t_{R}=11.4 min.

ESI-MS: m_{exact} (C₉₈H₁₄₀FN₂₅O₂₇PbS₂Si): 2417.9; m_{found}: m/z=807.2 [M+3H]³⁺, 1210.8 [M+2H]²⁺, 1613.5 [2M+3H]³⁺.

[^{nat}Pb]38

RP-HPLC (10-60% in 15 min): t_{R}=11.8 min.

ESI-MS: m_{exact} (C₉₉H₁₄₄FN₂₄O₂₆PbS₂Si⁺): 2404.0; m_{found}: m/z=801.7 [M+3H]³⁺, 1202.0 [M+2H]²⁺, 1603.4 [2M+3H]³⁺.

[^{nat}Pb]54

RP-HPLC (10-60% in 15 min): t_{R}=11.6 min.

ESI-MS: m_{exact} (C₁₀₁H₁₄₇FN₂₅O₂₇PbS₂Si⁺): 2461.0; m_{found}: m/z=616.2 [M+4H]⁴⁺, 821.2 [M+3H]³⁺, 1231.5 [M+2H]²⁺, 1641.7 [2M+3H]³⁺.

[^{nat}Pb]61

RP-HPLC (10-60% in 15 min): t_{R}=10.6 min.

ESI-MS: m_{exact} (C₁₀₃H₁₅₇FN₂₇O₂₃PbS₂Si⁺): 2459.1; m_{found}: m/z=615.7 [M+4H]⁴⁺, 820.7 [M+3H]³⁺, 1230.5 [M+2H]²⁺.

### 4. Radiolabeling

### 4.1 ⁶⁸Ga-Labeling

⁶⁸Ga-labeling was done using an automated system (GallElut⁺ by Scintomics, Germany) as described previously [28]. Briefly, the ⁶⁸Ge/⁶⁸Ga-generator with SnO₂ matrix (IThemba LABS) was eluted with 1.0 M aqueous HCl, from which a fraction (1.25 mL) of approximately 80% of the activity (500-700 MBq), was transferred into a reaction vial (ALLTECH, 5 mL). The reactor was loaded before elution with 2-5 nmol of respective chelator conjugate in an aqueous 2.7 M HEPES solution (900 μL). After elution the vial was heated for 5 minutes at 95° C. Purification was done by passing the reaction mixture over a solid phase extraction cartridge (C 8 light, SepPak), which was purged with water (10 mL) and the product eluted with 50% aqueous ethanol (2 mL), phosphate buffered saline (PBS, 1 mL) and again water (1 mL). After removing ethanol in vacuo, purity of the radiolabelled compounds was determined by radio-TLC (ITLC-SG chromatography paper, mobile phase: 0.1 M trisodium citrate and 1:1 mixture (v/v) of 1 M ammonium acetate and methanol).

### 4.2 ¹⁸F-Labeling

For ¹⁸F-labeling a previously published procedure was applied [29].

### 4.3 ¹²⁵I-Labeling of TOC

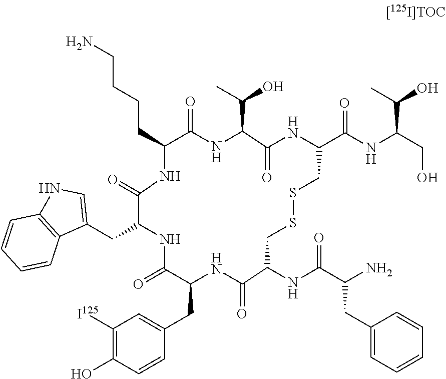

The reference ligand for in vitro studies [¹²⁵I]TOC was prepared according to a previously published procedure [30]. Briefly, 50-150 μg of the uniodinated precursor TOC were dissolved in 20 μL DMSO and 280 μL TRIS iodination buffer (25 mM TRIS-HCl, 0.4 mM NaCl, pH=7.5). After addition of 5.00 μL (15-20 MBq) [¹²⁵I]Nal (74 TBq/mmol, 3.1 GBq/mL, 40 mM NaOH, Hartmann Analytic, Braunschweig, Germany) the solution was transferred to a reaction vial, coated with 150 μg IodoGen®. The reaction was incubated for 15 min at RT and stopped by separation of the solution from the oxidant. The crude product of [¹²⁵I]l-TOC was purified by RP-HPLC [(20% to 50% B in 15 min): t_{R}=9.4 min] and the final, dissolved product was treated with 10 Vol-% of a 100 mM solution of Na-ascorbate in H₂O to prevent radiolysis.

### 5. In Vitro Experiments

### 5.1 Determination of IC₅₀

The SST₂-transfected CHO cells were cultivated in Dulbecco's Modified Eagle Medium/Nutrient Mixture F-12 (DMEM/F-12) with Glutamax-I (1:1) (Gibco), supplemented with 10% fetal calf serum (FCS) and maintained at 37° C. in a humidified 5% CO₂ atmosphere. For determination of the half maximal inhibitory concentration (IC₅₀), cells were harvested 24±2 hours before the experiment, seeded in 24-well plates (1.0×10⁵ cells) and incubated in 1 mL/well of culture medium. After removal of the culture medium, the cells were treated once with 300 μL of HBSS-B (Hank's balanced salt solution, Biochrom, Berlin, Germany, with addition of 1% bovine serum albumin (BSA)) and left with 200 μL HBSS-B. Next, 25 μL per well of solutions, containing either HBSS-B (control) or the respective ligand in increasing concentration (10⁻¹⁰-10⁻⁴ M in HBSS-B, were added with subsequent addition of 25 μL of

[¹²⁵I]TOC ([¹²⁵I]Tyr³-Octreotide) (1.0 nM) in HBSS-B. Each concentration is investigated in triplicate. After 60 min incubation at room temperature, the experiment was terminated by removal of the assay medium and consecutive rinsing with 300 μL of cold PBS. The media of both steps were combined in one fraction and represent the amount of unbound radioligand.

Afterwards, the cells were lysed with 300 μL of 1 M NaOH and united with the 300 μL 1 M NaOH of the following wash step. Quantification of bound and unbound radioligand was accomplished in a γ-counter.

### 5.2 Internalization

The AR42J cells were cultivated in RPMI Medium (Gibco), supplemented with 10% fetal calf serum (FCS) and 2 mM L-Glu and maintained at 37° C. in a humidified 5% CO₂ atmosphere. For internalization studies, AR42J cells were harvested 24±2 hours before the experiment and seeded in 24-well PLL-plates (2×10⁵ cells in 1 mL/well). Subsequent to the removal of the culture medium, the cells were washed once with 300 μL RPMI (5% BSA, 2 mM L-Glu) and left to equilibrate for at least 15 min at 37° C. in 200 μL RPMI (5% BSA, 2 mM L-Glu). Each well was treated with either 25 μL of either RPMI (5% BSA, 2 mM L-Glu) or a 10 μM TOC solution (1 μM assay concentration) for blockade. Next, 25 μL of a solution, containing the ¹⁸F-labeled SST ligand in a 20 nM concentration and also containing the reference ligand [¹²⁵I]TOC in a 1 nM concentration, was added and the cells incubated at 37° C. for 15, 30 and 60 min. For each investigated time point a separate well plate is prepared. The experiment was terminated by placing the 24-well plate on ice and consecutive removal of the medium. Each well was rinsed with 300 μL RPMI (2 mM L-Glu) and the fractions from these first two steps combined, representing the amount of free radioligand. Removal of surface bound activity was accomplished by incubation of the cells with 300 μL of ice-cold acid wash solution for 15 min and rinsed again with another 300 μL of ice-cold acid wash. The internalized activity was determined by incubation of the cells in 300 μL 1 M NaOH and the combination with the fraction of a subsequent wash step with 300 μL 1.0 M NaOH. Each experiment (control and blockade) was performed in triplicate. Free, surface bound and internalized activity was quantified in a γ-counter. Data were corrected for non-specific internalization and normalized to the specific-internalization observed for the radioiodinated reference compound.

### 5.3 Octanol-water Distribution Coefficient

Approximately 1 MBq of the labeled tracer was dissolved in 1 mL of a 1:1 mixture (by volumes) of phosphate buffered saline (PBS, pH 7.4) and n-octanol in an Eppendorf tube. After vigorous mixing of the suspension for 3 minutes at room temperature, the vial was centrifuged at 15000 g for 5 minutes (Biofuge 15, Heraus Sepatech, Osterode, Germany) and 100 μL aliquots of both layers were measured in a gamma counter. The experiment was repeated at least six times.

### 5.4 HSA Binding

### RIAC Method

A gel filtration column Superdex 75 Increase 10/300 GL (GE Healthcare, Uppsala, Sweden) was beforehand calibrated following the producer's recommendations with a commercially available gel filtration calibration kit (GE Healthcare, Buckinghamshire, UK) comprising conalbumin (MW: 75 kDa), ovalbumin (44 kDa), carbonic anhydrase (29 kDa), ribonuclease A (13.7 kDa) and aprotinin (6.5 kDa) as reference proteins of known molecular weight. AMSEC experiments were conducted using a constant flow rate of 0.8 mL/min at rt. A solution of HSA in PBS at physiological concentration (700 μM) was used as the mobile phase. SST₂ ligands were labelled as described with molar activities of 10-20 GBq/μmol. Probes of 1.0 MBq of the radioligand were injected directly from the labelling solution. HSA binding was expressed as an apparent molecular weight MW calculated from the retention time of the radioligand using the determined calibration curve.

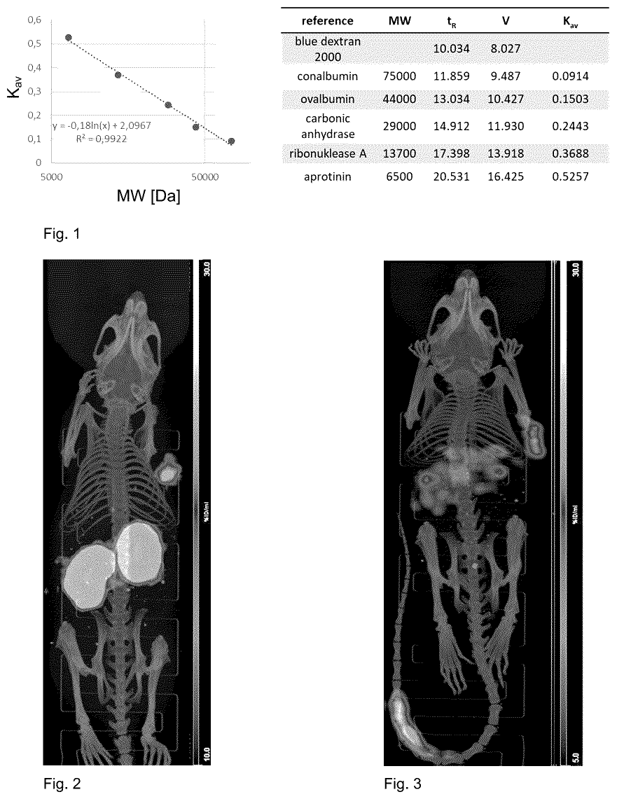

FIG. 1 shows the calibration plot of a Superdex 75 Increase gel filtration column using a low molecular weight gel filtration calibration kit in line with the data in the table below. MW: molecular weight. t_{R}: experimentally determined retention time. V: elution volume. Kₐᵥ: partition

For evaluation, experimentally determined retention times t_{R} are first converted into elution volumes Vₑ by multiplying with the flow rate and thereafter converted into partition coefficients Kₐᵥ following the equation

$${K}_{av} = \frac{{V}_{e} - {V}_{0}}{{V}_{c} - {V}_{0}}$$

where V₀ is the column void volume (8.027 mL) and V_{c} is the geometric column volume (24 mL). Using the equation given by the trend line plot of the column calibration

$${K}_{av} = - 0.18 ln ( MW ) + 2.0967$$

the apparent molecular weight MW is calculated as

$$MW = {e}^{\frac{2.0967 - {K}_{av}}{0.18}}$$

### 6. In Vivo Experiments

### 6.1 Mouse Model and Tumor Model

All animal experiments were conducted in accordance with general animal welfare regulations in Germany and the institutional guidelines for the care and use of animals. To establish tumor xenografts, AR42J cells (5×10⁶ cells/100 μL) were suspended in Dulbecco modified Eagle medium/Nutrition Mixture F-12 with Glutamax-1 (1:1) and inoculated subcutaneously onto the right shoulder of 8 weeks old, female CD1 nu/nu mice (Charles River, Sulzfeld, Germany). Mice were used for experiments when tumors had grown to a diameter of 5-9 mm (7-15 days after inoculation).

### 6.2 μPET/CT Imaging

Imaging experiments were conducted using a MILabs VECTor⁴ small-animal SPECT/PET/OI/CT. The resulting data were analyzed by the associated PMOD (version 4.0) software. Mice were anaesthetized with isoflurane and the ¹⁸F-labeled compounds were injected via the tail vein (0.05-0.25 nmol; 1-20 MBq). Mice were euthanized 1 h p.i. and blood samples for later biodistribution studies were taken by cardiac puncture before image acquisition. Static images were acquired with 45 min acquisition time using the HE-UHR-M collimator and a step-wise spiral bed movement. All images were reconstructed using the MILabs-Rec software (version 10.02) and a pixel-based Similarity-Regulated Ordered Subsets Expectation Maximization (SROSEM) algorithm, with a window-based scatter correction (20% below and 20% above the photopeak, respectively). Voxel size CT: 80 μm, 1.6 mm Gaussian blurring, with calibration factor in kBq/mL and decay correction. For blockade 20 μg of TOC was administered directly before tracer injection.

### 6.3 Biodistribution

Approximately 0.5-2.0 MBq (0.05-0.25 nmol) of the ¹⁸F-labeled SST₂-ligands were injected into the tail vein of AR42J tumor-bearing female CD1 nu/nu mice and sacrificed after 1 h post injection (n=3-5). Selected organs were removed, weighted and measured in a γ-counter.

### II. Results

The Tables 1, 2, and 3 summarize in vitro data of the herein described SST ligands.

TABLE 1
In vitro Data of reference ligands.

| Ligand | LogD_{pH=7.4} | IC₅₀ [nM] | HSA [kDa] |
|---|---|---|---|
| [Ga]1 | −2.02 ± 0.04 | 170 ± 2.05 | |
| [Ga]2 | −2.3 ± 0.09 | 162 ± 22.7 | |
| [Ga]3 | −1.87 ± 0.07 | 95.8 ± 8.41 | |
| [Ga]4 | −2.15 ± 0.11 | 86.8 ± 19.8 | |
| [Ga]5 | −2.25 ± 0.07 | 71.63 ± 13.98 | |
| [Ga]6 | −2.15 ± 0.09 | 149 ± 20.3 | |
| [Ga]7 | −2.14 ± 0.11 | 88.2 ± 26.8 | |
| [Ga]8 | −2.02 ± 0.04 | 58.5 ± 10.8 | |
| [Ga]9 | −2.02 ± 0.04 | 63.3 ± 1.75 | |
| [Ga]18 | −1.39 ± 0.04 | 7.15 ± 0.80 | 20.44 |

The reference ligands in table 1 exemplify the difficulties regarding the implementation of the radiohybrid structure (combination of chelator and SiFA-moiety). The incorporated chelator counter balances the high lipophilicity of the SiFA-moiety (Ligands [Ga]1 to [Ga]9). But the usage of SiFA-benzoic acid and DOTA or DOTAGA connected directly via the amino acid D-Dap as a trivalent linker impacted the target affinity (IC₅₀) negatively. Therefore, specific optimization steps had to be conducted, to develop ligands of sufficient target affinity, lipophilicity and a low affinity towards human serum albumine as defined in the claims and illustrated by the following examples.

TABLE 2
In vitro Data of ligands in accordance with the invention.

| Ligand | LogD_{pH=7.4} | IC₅₀ [nM] | HSA [kDa] |
|---|---|---|---|
| [Ga]19 | −2.11 ± 0.04 | 11.2 ± 1.04 | 21.17 |
| [Lu]19 | −1.85 ± 0.02 | 13.3 ± 1.97 | |
| [Ga]20 | −2.16 ± 0.04 | 12.7 ± 1.00 | 21.49 |
| [Lu]20 | −2.35 ± 0.04 | 11.0 ± 5.69 | 18.76 |
| [Ga]23 | −1.86 ± 0.06 | 23.6 ± 3.57 | 18.59 |
| [Pb]36 | −1.82 ± 0.05 | 6.57 ± 0.32 | 20.22 |
| 36 | −1.41 ± 0.07 | 7.05 ± 1.61 | |
| [Ga]37 | −1.92 ± 0.04 | 4.66 ± 0.53 | 17.60 |
| [Ga]39 | −2.20 ± 0.05 | 14.87 ± 1.22 | 24.96 |
| [Ga]40 | −2.24 ± 0.03 | 3.61 ± 0.29 | 19.25 |
| [Ga]41 | −1.58 ± 0.05 | 3.64 ± 0.64 | 15.48 |
| [Ga]42 | −2.41 ± 0.05 | 5.33 ± 0.78 | 17.97 |
| [Ga]43 | −2.29 ± 0.05 | 16.63 ± 5.89 | 20.69 |

TABLE 3
In vitro Data of ligands in accordance with the invention.

| Ligand | LogD_{pH=7.4} | IC₅₀ [nM] | HSA [kDa] |
|---|---|---|---|
| [Ga]24 | −1.92 ± 0.03 | 9.48 ± 1.96 | 8.77 |
| [Lu]24 | −1.90 ± 0.05 | 5.50 ± 0.76 | |
| [Pb]38 | −1.95 ± 0.03 | 4.67 ± 0.73 | 9.20 |
| [Ga]44 | −2.06 ± 0.08 | 5.45 ± 0.50 | 6.99 |
| [Lu]44 | −1.98 ± 0.05 | 3.86 ± 0.23 | |
| [Ga]45 | −2.10 ± 0.04 | 4.30 ± 0.06 | 7.76 |
| [Lu]45 | −2.10 ± 0.08 | 3.37 ± 0.35 | |
| [Ga]46 | −2.22 ± 0.03 | 4.79 ± 0.84 | 6.81 |
| [Lu]46 | −2.12 ± 0.05 | 4.07 ± 0.52 | |
| [Ga]47 | −2.30 ± 0.02 | 7.58 ± 0.58 | 7.66 |
| [Lu]47 | −2.29 ± 0.03 | 5.13 ± 0.51 | |
| [Ga]48 | −2.10 ± 0.05 | 6.10 ± 0.57 | 8.11 |
| [Lu]48 | −2.19 ± 0.05 | 5.50 ± 1.47 | |
| [Ga]49 | −2.16 ± 0.03 | 6.31 ± 0.99 | 7.26 |
| [Lu]49 | −2.24 ± 0.06 | 4.47 ± 0.95 | |
| [Ga]50 | −2.00 ± 0.12 | 3.01 ± 0.03 | 8.18 |
| [Lu]50 | −1.79 ± 0.02 | 3.72 ± 0.61 | 8.17 |
| [Ga]51 | −1.97 ± 0.01 | 3.56 ± 0.25 | 8.71 |
| [Lu]51 | −1.71 ± 0.05 | 3.09 ± 0.16 | 8.84 |
| [Ga]52 | −2.00 ± 0.02 | 3.69 ± 0.34 | 7.82 |
| [Lu]52 | −1.88 ± 0.05 | 3.51 ± 0.41 | 8.47 |
| [Ga]53 | −1.96 ± 0.01 | 3.40 ± 0.18 | 8.48 |
| [Lu]53 | −1.74 ± 0.03 | 3.36 ± 0.27 | 9.81 |
| [Pb]54 | −1.86 ± 0.04 | 4.24 ± 0.68 | |
| [Ga]55 | −1.69 ± 0.04 | 4.10 ± 0.54 | |
| [Ga]56 | −1.51 ± 0.03 | 4.00 ± 0.34 | |
| [Ga]57 | −2.25 ± 0.14 | 4.74 ± 0.22 | |
| [Ga]58 | −2.31 ± 0.04 | 4.41 ± 0.81 | |
| [Ga]59 | −2.14 ± 0.03 | 23.7 ± 1.50 | |
| [Ga]60 | −2.24 ± 0.05 | 4.15 ± 0.79 | |
| [Pb]61 | ± | 7.96 ± 0.28 | |

The Tables 4, 5, and 6 summarize the results of the in vivo mouse experiments. For all biodistribution studies, female CD1 nu/nu mice with AR42J tumor xenografts, were used and sacrificed 1 h post injection.

TABLE 4
Biodistribution data of [¹⁸F][^{nat}Ga]19 and [¹⁸F][^{nat}Pb]36.

| | [¹⁸F][^{nat}Ga]19 | [¹⁸F][^{nat}Pb]36 |
|---|---|---|
| | (n = 5) | (n = 5) |
| | % ID/g ± SD | % ID/g ± SD |
| Blood | 2.24 ± 0.30 | 1.30 ± 0.21 |
| Heart | 0.88 ± 0.11 | 0.71 ± 0.09 |
| Lung | 4.84 ± 0.98 | 2.42 ± 0.50 |
| Liver | 2.47 ± 0.14 | 8.47 ± 1.23 |
| Spleen | 0.93 ± 0.15 | 0.84 ± 0.17 |
| Pancreas | 12.47 ± 1.87 | 9.09 ± 1.79 |
| Stomach | 7.99 ± 2.08 | 4.32 ± 0.81 |
| Intestine | 1.39 ± 0.24 | 1.31 ± 0.21 |
| Kidneys | 131.06 ± 26.66 | 43.02 ± 6.22 |
| Adrenal glands | 2.56 ± 0.21 | 2.00 ± 0.27 |
| Muscle | 0.27 ± 0.04 | 0.47 ± 0.08 |
| Bone | 1.96 ± 0.38 | 3.38 ± 0.70 |
| Tumor | 36.27 ± 4.54 | 22.30 ± 3.85 |

TABLE 5
Biodistribution data and results of a blocking
study of [¹⁸F][^{nat}Ga]24.

| | | [¹⁸F][^{nat}Ga]24 |
|---|---|---|
| | [¹⁸F][^{nat}Ga]24 | Blocking |
| | (n = 4) | (n = 2) |
| | % ID/g ± SD | % ID/g ± SD |
| Blood | 0.66 ± 0.32 | 1.24 ± 0.61 |
| Heart | 0.34 ± 0.09 | 0.53 ± 0.00 |
| Lung | 2.33 ± 0.10 | 1.36 ± 0.13 |
| Liver | 1.05 ± 0.22 | 1.82 ± 0.22 |
| Spleen | 0.45 ± 0.05 | 0.33 ± 0.00 |
| Pancreas | 9.28 ± 0.57 | 1.07 ± 0.02 |
| Stomach | 4.95 ± 0.35 | 1.01 ± 0.06 |
| Intestine | 1.41 ± 0.19 | 0.71 ± 0.04 |
| Kidneys | 13.56 ± 3.58 | 18.24 ± 2.03 |
| Adrenal | 1.52 ± 0.46 | 0.54 ± 0.05 |
| glands | | |
| Muscle | 0.09 ± 0.04 | 0.21 ± 0.01 |
| Bone | 1.32 ± 0.19 | 0.77 ± 0.07 |
| Tumor | 23.94 ± 4.10 | 6.10 ± 0.02 |

TABLE 6
Biodistribution data of [¹⁸F][^{nat}Ga]45, [¹⁸F][^{nat}Ga]46
and [¹⁸F][^{nat}Ga]50.

| | [¹⁸F][^{nat}Ga]45 | [¹⁸F][^{nat}Ga]46 | [¹⁸F][^{nat}Ga]50 |
|---|---|---|---|
| | (n = 4) | (n = 4) | (n = 4) |
| | % ID/g ± SD | % ID/g ± SD | % ID/g ± SD |
| Blood | 0.53 ± 0.23 | 0.57 ± 0.24 | 0.50 ± 0.21 |
| Heart | 0.28 ± 0.04 | 0.30 ± 0.04 | 0.27 ± 0.03 |
| Lung | 3.34 ± 0.49 | 3.22 ± 0.53 | 3.73 ± 0.57 |
| Liver | 1.28 ± 0.11 | 0.83 ± 0.14 | 1.22 ± 0.18 |
| Spleen | 0.56 ± 0.10 | 0.57 ± 0.14 | 0.55 ± 0.06 |
| Pancreas | 11.80 ± 1.36 | 11.10 ± 2.61 | 17.26 ± 2.87 |
| Stomach | 7.50 ± 0.13 | 6.51 ± 1.64 | 9.54 ± 1.83 |
| Intestine | 1.27 ± 0.13 | 1.43 ± 0.35 | 1.84 ± 0.45 |
| Kidneys | 72.70 ± 7.68 | 12.64 ± 1.58 | 12.27 ± 3.40 |
| Adrenal | 1.54 ± 0.28 | 1.71 ± 0.38 | 2.39 ± 0.53 |
| glands | | | |
| Muscle | 0.11 ± 0.02 | 0.10 ± 0.02 | 0.10 ± 0.03 |
| Bone | 1.81 ± 0.07 | 1.21 ± 0.24 | 1.23 ± 0.41 |
| Tumor | 31.61 ± 4.96 | 23.40 ± 1.07 | 31.81 ± 2.49 |

The FIGS. 2, 3 and 4 show the MIPs of the PET/CT scans of AR42J tumor bearing female mice (CD1 nu/nu). To perform the static PET/CT scan, mice were euthanized 1 h p.i. and images were acquired with 45 min acquisition time.

FIG. 2 shows the PET/CT MIP of [18F][^{nat}Ga]19 in female AR42 tumor-bearing CD1 nu/nu mice from 10-30% ID/mL. Arrows indicate organs/volumes of interest: solid=tumor, points=kidney

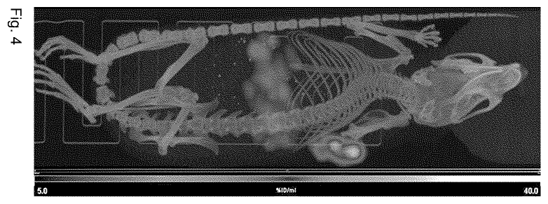

FIG. 3 shows the PET/CT MIP of [18F][^{nat}Ga]46 in female AR42 tumor-bearing CD1 nu/nu mice from 5-30% ID/mL. Arrows indicate organs/volumes of interest: solid=tumor, points=overlap of kidneys and organs that express the receptor on a physiological level (e.g. stomach and pancreas).

FIG. 4 shows the PET/CT MIP of [18F][^{nat}Ga]50 in female AR42 tumor-bearing CD1 nu/nu mice from 5-40% ID/mL. Arrows indicate organs/volumes of interest: solid=tumor, points=overlap of kidneys and organs that express the receptor on a physiological level (e.g. stomach and pancreas).

### REFERENCES

1. Kaltsas G A, Besser G M, Grossman A B. The diagnosis and medical management of advanced neuroendocrine tumors [eng]. Endocr Rev. 2004; doi:10.1210/er.2003-0014.
2. Modlin I M, Lye K D, Kidd M. A 5-decade analysis of 13,715 carcinoid tumors [eng]. Cancer. 2003; doi:10.1002/cncr.11105.
3. Taal B G, Visser 0. Epidemiology of neuroendocrine tumours [eng]. Neuroendocrinology. 2004; doi:10.1159/000080731.
4. Oronsky B, Ma P C, Morgensztern D, Carter C A. Nothing But NET: A Review of Neuroendocrine Tumors and Carcinomas [eng]. Neoplasia. 2017; doi:10.1016/j.neo.2017.09.002.
5. Papotti M, Croce S, Bellò M, Bongiovanni M, Allia E, Schindler M, et al. Expression of somatostatin receptor types 2, 3 and 5 in biopsies and surgical specimens of human lung tumours. Correlation with preoperative octreotide scintigraphy [eng]. Virchows Arch. 2001; doi:10.1007/s004280100494.
6. GOnther T, Tulipano G, Dournaud P, Bousquet C, Csaba Z, Kreienkamp H-J, et al. International Union of Basic and Clinical Pharmacology. C V. Somatostatin Receptors: Structure, Function, Ligands, and New Nomenclature [eng]. Pharmacol Rev. 2018; doi:10.1124/pr.117.015388.
7. Hoyer D, Bell G I, Berelowitz M, Epelbaum J, Feniuk W, Humphrey P P A, et al. Classification and nomenclature of somatostatin receptors. Trends in Pharmacological Sciences. 1995; doi:10.1016/s0165-6147(00)88988-9.
8. Reubi J C, Waser B, SchaerJC, Laissue J A. Somatostatin receptor sstl-sst5 expression in normal and neoplastic human tissues using receptor autoradiography with subtype-selective ligands [eng]. Eur J Nucl Med. 2001; doi:10.1007/s002590100541.
9. Taniyama Y, Suzuki T, Mikami Y, Moriya T, Satomi S, Sasano H. Systemic distribution of somatostatin receptor subtypes in human: an immunohistochemical study [eng]. Endocr J. 2005; doi:10.1507/endocrj.52.605.
10. Patel Y C. Somatostatin and its receptor family [eng]. Front Neuroendocrinol. 1999; doi:10.1006/frne.1999.0183.
11. Xu C, Zhang H. Somatostatin receptor based imaging and radionuclide therapy [eng]. Biomed Res Int. 2015; doi:10.1155/2015/917968.
12. Tredwell M, Gouverneur V. 18F labeling of arenes [eng]. Angew Chem Int Ed Engl. 2012; doi:10.1002/anie.201204687.
13. Yusubov M S, Svitich D Y, Yoshimura A, Nemykin V N, Zhdankin V V. 2-Iodoxybenzoic acid organosulfonates: preparation, X-ray structure and reactivity of new, powerful hypervalent iodine(V) oxidants [eng]. Chem Commun (Camb). 2013; doi:10.1039/c3cc47090c.
14. Bernard-Gauthier V, Wangler C, Schirrmacher E, Kostikov A, Jurkschat K, Wängler B, et al. ¹⁸F-labeled silicon-based fluoride acceptors: potential opportunities for novel positron emitting radiopharmaceuticals [eng]. Biomed Res Int. 2014; doi:10.1155/2014/454503.
15. Bernard-Gauthier V, Bailey J J, Liu Z, Wangler B, Wangler C, Jurkschat K, et al. From Unorthodox to Established: The Current Status of (18)F-Trifluoroborate- and (18)F-SiFA-Based Radiopharmaceuticals in PET Nuclear Imaging [eng]. Bioconjug Chem. 2016; doi:10.1021/acs.bioconjchem.5b00560.
16. Schirrmacher R, Bradtmoller G, Schirrmacher E, Thews O, Tillmanns J, Siessmeier T, et al. 18F-labeling of peptides by means of an organosilicon-based fluoride acceptor [eng]. Angew Chem Int Ed Engl. 2006; doi:10.1002/anie.200600795.
17. Iovkova L, Könning D, Wangler B, Schirrmacher R, Schoof S, Arndt H-D, et al. SiFA-Modified Phenylalanine: A Key Compound for the Efficient Synthesis of 18F-Labelled Peptides. Eur. J. Inorg.

Chem. 2011; doi:10.1002/ejic.201100142.
18. Litau S, Niedermoser S, Vogler N, Roscher M, Schirrmacher R, Fricker G, et al. Next Generation of SiFAlin-Based TATE Derivatives for PET Imaging of SSTR-Positive Tumors: Influence of Molecular Design on In Vitro SSTR Binding and In Vivo Pharmacokinetics [eng]. Bioconjug Chem. 2015; doi:10.1021/acs.bioconjchem.5b00510.
19. Niedermoser S, Chin J, Wangler C, Kostikov A, Bernard-Gauthier V, Vogler N, et al. In Vivo Evaluation of ¹⁸F-SiFAlin-Modified TATE: A Potential Challenge for ⁶⁸Ga-DOTATATE, the Clinical Gold Standard for Somatostatin Receptor Imaging with PET [eng]. J Nucl Med. 2015; doi:10.2967/jnumed.114.149583.
20. Ilhan H, Todica A, Lindner S, Boening G, Gosewisch A, Wsngler C, et al. First-in-human 18F-SiFAlin-TATE PET/C T for NET imaging and theranostics [eng]. Eur J Nucl Med Mol Imaging. 2019; doi:10.1007/s00259-019-04448-8.
21. Goffin K. Al18F-NOTA-octreotide and 18F-SiFAlin-TATE: two ‘new kids on the block’ in somatostatin receptor imaging [eng]. EurJ Nucl Med Mol Imaging. 2019; doi:10.1007/s00259-019-04474-6.
22. Ilhan H, Lindner S, Todica A, Cyran C C, Tiling R, Auernhammer C J, et al. Biodistribution and first clinical results of 18F-SiFAlin-TATE PET: a novel 18F-labeled somatostatin analog for imaging of neuroendocrine tumors [eng]. EurJ Nucl Med Mol Imaging. 2019; doi:10.1007/s00259-019-04501-6.
23. Lindner S, Simmet M, Gildehaus F J, Jurkschat K, Wangler C, Wangler B, et al. Automated production of 18FSiTATE on a Scintomics GRP™ platform for PET/CT imaging of neuroendocrine tumors [eng]. Nucl Med Biol. 2020; doi:10.1016/j.nucmedbio.2020.07.008.
24. Wurzer A, DiCarlo D, Schmidt A, Beck R, Eiber M, Schwaiger M, et al. Radiohybrid ligands: a novel tracer concept exemplified by 18F- or 68Ga-labeled rhPSMA-inhibitors [eng]. J Nucl Med. 2019; doi:10.2967/jnumed.119.234922.
25. Wangler C, Waser B, Alke A, lovkova L, Buchholz H-G, Niedermoser S, et al. One-step ¹⁸F-labeling of carbohydrate-conjugated octreotate-derivatives containing a silicon-fluoride-acceptor (SiFA): in vitro and in vivo evaluation as tumor imaging agents for positron emission tomography (PET) [eng]. Bioconjug Chem. 2010; doi:10.1021/bc100316c.
26. Fujii N, Otaka A, Funakoshi S, Bessho K, Watanabe T, Akaji K, et al. Studies on peptides. CLI. Syntheses of cystine-peptides by oxidation of S-protected cysteine-peptides with thallium(III) trifluoroacetate [eng]. Chem Pharm Bull. 1987; doi:10.1248/cpb.35.2339.
27. Andreu D, Albericio F, Sole N A, Munson M C, Ferrer M, Barany G. Formation of disulfide bonds in synthetic peptides and proteins [eng]. Methods Mol Biol. 1994; doi:10.1385/0-89603-273-6:91.
28. Notni J, Pohle K, Wester H-J. Comparative gallium-68 labeling of TRAP-, NOTA-, and DOTA-peptides: practical consequences for the future of gallium-68-PET [eng]. EJNMMI Res. 2012; doi:10.1186/2191-219X-2-28.
29. Wangler C, Niedermoser S, Chin J, Orchowski K, Schirrmacher E, Jurkschat K, et al. One-step (18)F-labeling of peptides for positron emission tomography imaging using the SiFA methodology [eng].

Nat Protoc. 2012; doi:10.1038/nprot.2012.109.
30. Wester H-J, Schottelius M, Scheidhauer K, Reubi J-C, Wolf I, Schwaiger M. Comparison of radioiodinated TOC, TOCA and Mtr-TOCA: the effect of carbohydration on the pharmacokinetics [eng]. Eur J Nucl Med Mol Imaging. 2002; doi:10.1007/s00259-001-0669-1.

1. A compound of formula (I) or a salt thereof: 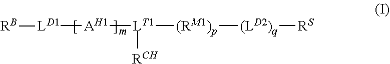
wherein:
R^{B} is a binding motif which is able to bind to a somatostatin receptor;
L^{D1} is a divalent linking group;
m is an integer of 2 to 6;
A^{H1} is, independently for each occurrence, an amino acid unit derived from a hydrophilic amino acid which comprises, in addition to its —NH₂ and its —COOH functional group, a further hydrophilic functional group,
and wherein one of the m units A^{H1} may further carry a hydrophilic unit other than an amino acid bound to its hydrophilic functional group;
L^{T1} is a trivalent linking group;
R^{CH} is a chelating group which optionally contains a chelated radioactive or non-radioactive cation;
R^{M1} is a hydrophilic modifying group, and p is 0 or 1;
L^{D2} is a divalent linking group, and q is 0 or 1; and
R^{S} is a silicon-fluoride acceptor (SiFA) group of one of the following formulae, which comprises a silicon atom and a fluorine atom, and which can be labeled with ¹⁸F by isotopic exchange of ¹⁹F by ¹⁸F or which is labeled with ¹⁸F:
if p is 1, R^{S} is a group of formula (S-3) or a group of formula (S-4): 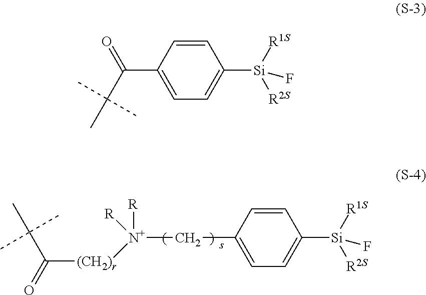
wherein
r is 1, 2 or 3, s in —(CH₂)ₛ— is an integer of 1 to 6,
the groups R are, independently, H or C1 to C6 alkyl, and
R^{1S} and R^{2S} are independently from each other a linear or branched C3 to C10 alkyl group; and wherein the dashed line marks a bond which attaches the group to the remainder of the compound; and
if p is 0, R^{S} is a group of formula (S-4) as defined above.

2. The compound or salt of claim 1, wherein the binding motif R^{B} comprises a group which can be derived from a receptor agonist or receptor antagonist selected from Tyr³,Thr⁸-Octreotide (TATE), Tyr³-Octreotide (TOC), Thr⁸-Octreotide (ATE); 1-Nal³-Octreotide (NOC), 1-Nal³,Thr⁸-octreotide (NOCATE), BzThi³-octreotide (BOC), BzThi³,Thr⁸-octreotide (BOCATE), JR11, BASS, and KE121.

3. The compound or salt of claim 1 or 2, wherein the binding motif R^{B} comprises a group of the formula (B-1) or (B-2): 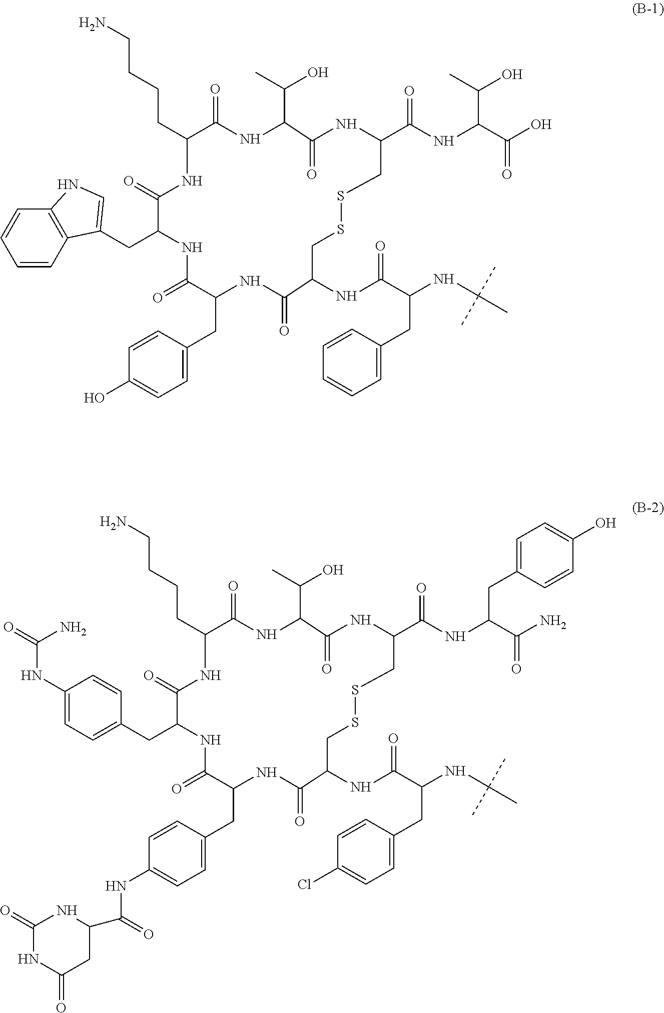
wherein the dashed line marks a bond which attaches the group to the remainder of the compound.

4. The compound or salt of any of claims 1 to 3, wherein the chelating group R^{CH} comprises a group which can be derived from a chelating agent selected from diethylenetriaminepentamethylenephosphonic acid (EDTMP) and its derivatives, diethylenetriaminepentaacetic acid (DTPA) and its derivatives, bis(carboxymethyl)-1,4,8,11-tetraaza-bicyclo[6.6.2] hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino],pentyl]-N-[5-[[4-[5-aminopentyl-(hydroxy)amino]-4-oxobutanoyl]-amino]pentyl]-N-hydroxybutandiamide (DFO) and derivatives thereof, 1,4,7,10-tetraazacyclododecane-1,7-diacetic acid (DO2A), 1,4,7,10-tetraazacyclododecan-N,N′,N″,N′″-tetraacetic acid (DOTA), 2-[1,4,7,10-tetraazacyclododecane-4,7,10-triacetic acid]-pentanedioic acid (DOTAGA or DOTA-GA), 1,4,7,10-tetrakis(carbamoylmethyl)-1,4,7,10-tetraazacyclododecane (DOTAM), N,N′-dipyridoxylethylendiamine-N,N′-diacetate-5,5′-bis(phosphat) (DPDP), diethylenetriaminepentaacetic acid (DTPA), ethylenediamine-N,N′-tetraacetic acid (EDTA), ethyleneglykol-O,O-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclodecan-4,7,10-triacetate (HP-DOA3), 6-hydrazinyl-N-methylpyridine-3-carboxamide (HYNIC), 1,4,7-triazacyclononan-1-succinic acid-4,7-diacetic acid (NODASA), 1-(1-carboxy-3-carboxypropyl)-4,7-(carboxy)-1,4,7-triazacyclononane (NODAGA), 1,4,7-triazacyclononanetriacetic acid (NOTA), 4,11-bis(carboxymethyl)-1,4,8,11-tetraazabicyclo[6.6.2]hexadecane (TE2A), 1,4,8,11-tetraazacyclododecane-1,4,8,11-tetraacetic acid (TETA), terpyridine-bis(methyleneamine) tetraacetic acid (TMT), 1,4,7,10-tetraazacyclotridecan-N,N′,N″,N′″-tetraacetic acid (TRITA), and triethylenetetraaminehexaacetic acid (TTHA), N,N′-bis[(6-carboxy-2-pyridil)methyl]-4,13-diaza-18-crown-6 (H₂macropa), 4-amino-4-{2-[(3-hydroxy-1,6-dimethyl-4-oxo-1,4-dihydro-pyridin-2-ylmethyl)-carbamoyl]-ethyl} heptanedioic acid bis-[(3-hydroxy-1,6-dimethyl-4-oxo-1,4-dihydro-pyridin-2-ylmethyl)-amide] (THP), 1,4,7-triazacyclononane-1,4,7-tris[methylene(2-carboxyethyl)phosphinic acid (TRAP), 2-(4,7,10-tris(2-amino-2-oxoethyl)-1,4,7,10-tetraazacyclododecan-1-yl)acetic acid (DO3AM), and 1,4,7,10-tetraazacyclododecane-1,4,7,10-tetrakis[methylene(2-carboxyethylphosphinic acid)](DOTPI), S-2-(4-isothiocyanatobenzyl)-1,4,7,10-tetraazacyclododecane tetraacetic acid, hydrazinonicotinic acid (HYNIC), 6-amino-6-methylperhydro-1,4-diazepine-N,N,N′,N′-tetraacetic acid (AAZTA) and derivatives thereof, such as (6-pentanoic acid)-6-(amino)methyl-1,4-diazepine triacetate (DATA), pentadeca-1,4,7,10,13-penta-aminopentaacetic acid (PEPA), hexadeca-1,4,7,10,13,16-hexaamine-hexaacetic acid (HEHR), 4-{[bis(phosphonomethyl)) carbamoyl] methyl}-7,10-bis(carboxymethyl)-1,4,7,10-tetraazacyclododec-1-yl) acetic acid (BPAMD), N-(4-{[bis (phosphonomethyl)) carbamoyl] methyl}-7,10-bis (carboxymethyl)-nona-1,4,7-triamine triacetic acid (BPAM), 1,2-[{6-(carboxylate) pyridin-2-yl} methylamine] ethane (DEDPA, H₂DEDPA), deferoxamine (DFO) and its derivatives, deferiprone, (4-acetylamino-4-yl) {2-[(3-hydroxy-1,6-dimethyl-4-oxo-1,4-dihydro-pyridin-2-ylmethyl)-carbamoyl]-ethyl}-heptanedioic acid bis-[(3-hydroxy-1,6-dimethyl-4-oxo-1,4-dihydro-pyridin-2-ylmethyl)-amide](CP256) and its derivatives such as YM103; tetraazycyclodecane-phosphinic acid (TEAP), 6-amino-6-methylperhydro-1,4-diazepine-N,N,N′,N′-tetraacetic acid (AAZTA); 1-N-(4-aminobenzyl)-3,6,10,13,16,19-hexaazabicyclo [6.6.6]-eicosane-1,8-diamine (SarAr), 6,6′-[{9-hydroxy-1,5-bis-(methoxycarbonyl)-2,4-di(pyridin-2-yl)-3,7-diazabicyclo[3.3.1]nonane-3,7-diyl}bis(methylene)]dipicolinic acid (H₂bispa²), 1,2-[{6-(carboxylato)pyridin-2-yl}methylamino]-ethane (H₂dedpa), N,N′-bis(6-carboxy-2-pyridylmethyl)-ethylenediamine-N,N′-diacetic acid (H₄octapa), N,N′-bis(2-hydroxy-5-sulfonylbenzyl)-N,N′-bis-(2-methylpyridyl)ethylenediamine (HeSbbpen) and derivatives thereof, triethylenetetramine-N,N,N′,N″,N′″,N′″-hexaacetic (TTHA), 2-aminomethylpiperidine triacetic acid (2-AMPTA) and derivatives thereof, such as 2-(N-(2-Hydroxybenzyl)aminomethyl)piperidine (2-AMPTA-HB) further functionalized derivative of 2-AMPTA with additional functional groups suitable for conjugation to peptidic structures, 4-nitro-2-hydroxybenzyl-2-{[(6)-trans-2-[benzyl(carboxymethyl)amino]cyclohexyl](carboxymethyl)amino}acetic acid (RESCA) and derivatives thereof, and 6-carboxy-1,4,8,11-tetraazaundecane (N₄) and derivatives thereof, and wherein the chelating group optionally comprises a chelated radioactive or non-radioactive cation.

5. The compound or salt of claim 4, wherein the chelating group R^{CH} comprises a group which can be derived from a chelating agent selected from DOTA, DOTAGA, DOTAM, DO3AM, NOTA and NODAGA, and wherein the chelating group optionally comprises a chelated radioactive or non-radioactive cation.

6. The compound or salt of any of claims 1 to 5, wherein R^{CH} is a group of the formula (CH-1), (CH-2) or (CH-3) which optionally contains a chelated radioactive or non-radioactive cation: 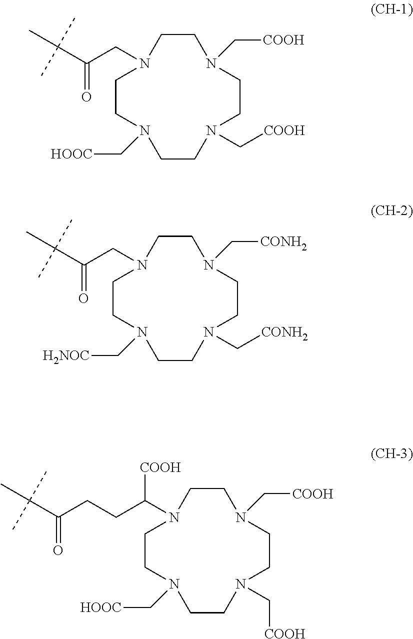
wherein the dashed line marks a bond which attaches the group to the remainder of the compound.

7. The compound or salt of any of claims 1 to 6, wherein the group of formula (S-3) is a group of formula (S-3a) and the group of formula (S-4) is a group of formula (S-4a): 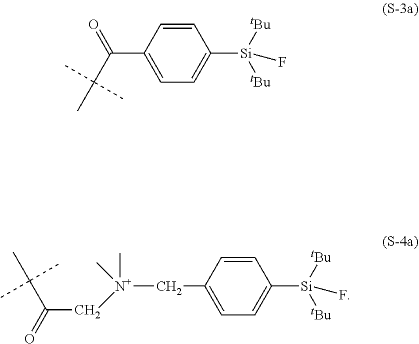
wherein ^{t}Bu indicates a tert-butyl group and the dashed line marks a bond which attaches the group to the remainder of the compound.

8. The compound or salt of any of claims 1 to 7, wherein the further hydrophilic functional group of the amino acid units A^{A1} is independently selected from —NH₂, —COOH, —NH—C(═NH)—NH₂, —C(═O)NH₂, —NH—C(═O)—NH₂, —OH and —P(═O)(OH)₂.

9. The compound or salt of any of claims 1 to 8, wherein the amino acid units A^{H1} are independently selected from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gin) unit, serine (Ser) unit, citrulline (Cit) unit and a phosphonomethylalanine (Pma) unit.

10. The compound or salt of any of claims 1 to 9, wherein p is 1 and the hydrophilic modifying group R^{M1} is an amino acid unit which is selected from a diaminopropionic acid (Dap) unit, 2,4-diaminobutanoic acid (Dab) unit, ornithine (Orn) unit and a lysine (Lys) unit, more preferably a diaminopropionic acid unit, or wherein p is 0.

11. The compound or salt of any of claims 1 to 10, wherein q is 1 and the divalent linking group L^{D2} is a group of the formula (L-2), or q is 0:
-[A^{L2}]_{w}- (L-2)
wherein
A^{L2} is, independently for each occurrence if w is more than 1, an amino acid unit; and
w is 1 to 5, preferably 1 to 3, more preferably 1 or 2.

12. The compound or salt of any of claims 1 to 11, which is a compound of formula (I.2) or a salt thereof: 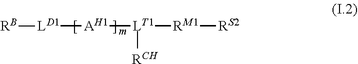
wherein
R^{B}, L^{D1}, A^{H1} m L^{T1}, R^{CH}, and R^{M1} are defined as in the preceding claims; and
R^{S2} is a group of the formula (S-3): 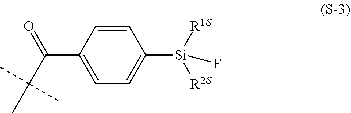
wherein
R^{1S} and R^{2S} are independently from each other a linear or branched C3 to C10 alkyl group;
and wherein the dashed line marks a bond which attaches the group to the remainder of the compound.

13. The compound or salt of any of claims 1 to 12, which is a compound of formula (I.4) or (1.5) or a salt of these: 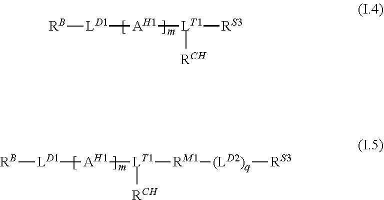
wherein
R^{B}, L^{D1}, A^{A1} m L^{T1}, R^{M1}, L^{D2}, q and R^{CH} are defined as in the preceding claims; and
R^{S3} is a group of the formula (S-4): 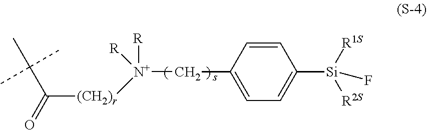
wherein
r is 1, 2 or 3, s is an integer of 1 to 6,
R is, independently, H or C1 to C6 alkyl,
R^{1S} and R^{2S} are independently from each other a linear or branched C3 to C10 alkyl group;
and wherein the dashed line marks a bond which attaches the group to the remainder of the compound.

14. The compound or salt of any of claim 1, 10 or 11, wherein R^{B} comprises a group which can be derived from a receptor agonist or receptor antagonist selected from Tyr³,Thr⁸-Octreotide (TATE), Tyr³-Octreotide (TOC), Thr⁸-Octreotide (ATE); 1-Nal³-Octreotide (NOC), 1-Nal³,Thr⁸-octreotide (NOCATE), BzThi³-octreotide (BOC), BzThi³,Thr⁸-octreotide (BOCATE), JR11, BASS, and KE121;
R^{CH} comprises a group which can be derived from a chelating agent selected from DOTA, DOTAGA, DOTAM, DO3AM, NOTA and NODAGA, and wherein the chelating group optionally comprises a chelated radioactive or non-radioactive cation;
A^{H1} is selected, independently for each occurrence, from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, serine (Ser) unit, citrulline (Cit) unit and a phosphonomethylalanine (Pma) unit.

15. A pharmaceutical or diagnostic composition comprising or consisting of one or more compounds or salts of any of claims 1 to 14.
